(19) United States
(12) Patent Application Publication
(10) Pub. No.: US 2016/0009693 A1
(43) Pub. Date: Jan. 14, 2016

(54) HERBICIDAL COMPOUNDS

(71) Applicant: SYNGENTA LIMITED, Surrey, Guildford Surrey (GB)

(72) Inventors: Glynn MITCHELL, Bracknell Berkshire (GB); Olivia Anabelle SAGEOT, Bracknell Berkshire (GB); Linda Hazel CURLEY, Bracknell Berkshire (GB)

(73) Assignee: Syngenta Limited, Guildford (GB)

(21) Appl. No.: 14/771,700

(22) PCT Filed: Mar. 8, 2014
(86) PCT No.: PCT/EP2014/054382

§ 371 Date: Aug. 31, 2015

(30) Foreign Application Priority Data
Mar. 8, 2013 (GB) 1304238.7

(51) Int. Cl. C07D 403/12; C07D 253/075; A01N 43/707; A01N 43/713; A01N 43/653; C07D 401/14; C07D 401/06

(52) U.S. Cl. CPC C07D 403/12; C07D 401/14; C07D 253/075; C07D 401/06; A01N 43/713; A01N 43/653; A01N 43/707

(57) ABSTRACT

The present invention relates to compounds of Formula (I), or an agronomically acceptable salt of said compounds wherein A¹, A², A³, X¹, X², R¹, R² and R⁴ are as defined herein. The invention further relates to herbicidal compositions which comprise a compound of Formula (I), to their use for controlling weeds, in particular in crops of useful plants, and to intermediates used to synthesise said compounds.

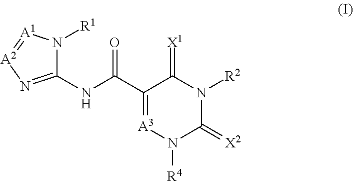

The present invention relates to novel herbicidal compounds, to processes for their preparation, to herbicidal compositions which comprise the novel compounds, and to their use for controlling weeds, in particular in crops of useful plants, or for inhibiting plant growth.

Herbicidal N-(tetrazol-5-yl)- and N-(triazol-5-yl)-arylcarboxamides are disclosed in WO 2012/028579. 6-Acyl-1,2,4-triazine-3,5-dione derivatives are disclosed in WO 2012/002096. Herbicidal 6-oxo-1,6-dihydropyrimidin-5-carboxamides and 2-oxo-1,2-dihydropyridin-3-carboxamides are disclosed in EP 2562174. The present invention provides further herbicidal derivatives. Thus, according to the present invention there is provided a compound of Formula (I):

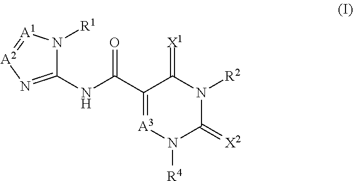

or an agronomically acceptable salt thereof,

wherein:—

X¹ and X² are independently selected from the group consisting of O and S;

A¹ and A² are independently selected from CH and N, wherein A¹ and A² are not both CH;

A³ is N or CR⁵;

wherein

R¹ is selected from the group consisting of hydrogen, C₁-C₆alkyl-, C₁-C₆ haloalkyl- and C₁-C₆alkoxy-C₁-C₃alkyl-;

R² is selected from the group consisting of hydrogen, C₁-C₆alkyl-, C₁-C₆haloalkyl-, C₁ -C₃alkoxy-C₁-C₃alkyl-, C₁ -C₃alkoxy-C₂-C₃alkoxy-C₁ -C₃-alkyl-, C₁-C₃haloalkoxy-C₁-C₃-alkyl-, (C₁-C₃ alkylsulfonyl-C₁-C₃ alkylamino)-C₁-C₃alkyl-, (C ₁ -C₃alkylsulfonyl-C₃-C₄cycloalkylamino)-C₁ -C₃alkyl-, C₁-C₆alkylcarbonyl-C₁-C₃alkyl-, C₃-C₆cycloalkyl-C₂-C₆alkenyl-, C₂-C₆alkynyl-,

C₂-C₆-alkenyl-, C₂-C₆-haloalkenyl-, cyano-C₁-C₆-alkyl-, arylcarbonyl-C₁-C₃-alkyl-, aryl-C₁-C₆alkyl-, aryloxy-C₁-C₆alkyl (wherein said aryl groups may be optionally substituted with one or more substituents from the group consisting of halo, C₁-C₃-alkoxy, C₁-C₃-alkyl, C₁-C₃ haloalkyl), and a three- to ten- membered mono- or bicyclic ring system, which may be aromatic, saturated or partially saturated and can contain from 1 to 4 heteroatoms each independently selected from the group consisting of nitrogen, oxygen and sulphur the ring system being optionally substituted by one or more substituents selected from the group consisting of nitro, cyano, halogen, C₁-C₃alkyl-, C₁-C₃haloalkyl-, C₂-C₃alkenyl-, C₂-C₃alkynyl-, C₁-C₃alkoxy-, C₁-C₃alkoxy-C₁-C₃alkyl-, C₁-C₃alkoxy-C₂-C₃alkoxy-, C₁-C₃alkoxy-C₂-C₃alkoxy-C₁-C₃-alkyl-, C₁-C₃ haloalkoxy, C₁-C₆alkyl-S(O)p- and C₁-C₆haloalkyl-S(O)p-;

R⁴ is selected from the group consisting of hydrogen, C₁-C₆alkyl-, C₁-C₆haloalkyl-, C₁-C₆alkoxy-C₁-C₆alkyl-, C₁-C₆haloalkoxy-C₁-C₆alkyl-, C₁-C₆alkoxy-C₁-C₆alkoxy-C₁-C₆alkyl-, C₂-C₆alkenyl-, C₂-C₆haloalkenyl-, C₂-C₆alkynyl-, C₂-C₆haloalkynyl- and C₃-C₆ cycloalkyl-;

R⁵ is selected from the group consisting of hydrogen, hydroxyl, C₁-C₆alkyl-, C₁-C₆haloalkyl- and C₁-C₆alkoxy-C₁-C₃alkyl-; and

p=0, 1 or 2.

Alkyl groups having a chain length of from 1 to 6 carbon atoms include, for example, methyl (Me, CH₃), ethyl (Et, C₂H₅), n-propyl (n-Pr), isopropyl (i-Pr), n-butyl (n-Bu), isobutyl (i-Bu), sec-butyl and tent-butyl (t-Bu).

Alkenyl groups having a chain length of from 2 to 6 carbon atoms include, for example, —CH═CH₂ (vinyl) and —CH₂—CH═CH₂ (allyl).

Alkynyl groups having a chain length of from 2 to 6 carbon atoms include, for example, —C≡CH (ethynyl) and —CH₂—C≡CH (propargyl).

Halogen (or halo) encompasses fluorine, chlorine, bromine or iodine. The same correspondingly applies to halogen in the context of other definitions, such as haloalkyl or halophenyl.

Haloalkyl groups having a chain length of from 1 to 6 carbon atoms are, for example, fluoromethyl, difluoromethyl, trifluoromethyl, chloromethyl, dichloromethyl, trichloromethyl, 2,2,2-trifluoroethyl, 2-fluoroethyl, 2-chloroethyl, pentafluoroethyl, 1,1-difluoro-2,2,2-trichloroethyl, 2,2,3,3-tetrafluoroethyl and 2,2,2-trichloroethyl, heptafluoro-n-propyl and perfluoro-n-hexyl.

Alkoxy groups preferably have a chain length of from 1 to 6 carbon atoms. Alkoxy is, for example, methoxy, ethoxy, propoxy, isopropoxy, n-butoxy, isobutoxy, sec-butoxy or tert-butoxy or a pentyloxy or hexyloxy isomer, preferably methoxy and ethoxy. It should also be appreciated that two alkoxy substituents present on the same carbon atom may be joined to form a spiro group. Thus, the methyl groups present in two methoxy substituents may be joined to form a spiro 1,3 dioxolane substituent, for example. Such a possibility is within the scope of the present invention.

Haloalkoxy is, for example, fluoromethoxy, difluoromethoxy, trifluoromethoxy, 2,2,2-trifluoroethoxy, 1,1,2,2-tetrafluoroethoxy, 2-fluoroethoxy, 2-chloroethoxy, 2,2-difluoroethoxy or 2,2,2-trichloroethoxy, preferably difluoromethoxy, 2-chloroethoxy or trifluoromethoxy.

C₁-C₆alkyl-S- (alkylthio) is, for example, methylthio, ethylthio, propylthio, isopropylthio, n-butylthio, isobutylthio, sec-butylthio or tert-butylthio, preferably methylthio or ethylthio.

C₁-C₆alkyl-S(O)- (alkylsulfinyl) is, for example, methylsulfinyl, ethylsulfinyl, propylsulfinyl, isopropylsulfinyl, n-butylsulfinyl, isobutylsulfinyl, sec-butylsulfinyl or tert-butylsulfinyl, preferably methylsulfinyl or ethylsulfinyl.

C₁-C₆alkyl-S(O)₂- (alkylsulfonyl) is, for example, methylsulfonyl, ethylsulfonyl, propylsulfonyl, isopropylsulfonyl, n-butylsulfonyl, isobutylsulfonyl, sec-butylsulfonyl or tert-butylsulfonyl, preferably methylsulfonyl or ethylsulfonyl.

Alkylamino is, for example, methylamino, ethylamino, n-propylamino, isopropylamino or a butylamino isomer. Dialkylamino is, for example, dimethylamino, methylethylamino, diethylamino, n-propylmethylamino, dibutylamino or diisopropylamino. Preference is given to alkylamino groups having a chain length of from 1 to 4 carbon atoms.

Alkoxyalkyl groups preferably have from 1 to 6 carbon atoms. Alkoxyalkyl is, for example, methoxymethyl, methoxyethyl, ethoxymethyl, ethoxyethyl, n-propoxymethyl, n-propoxyethyl, isopropoxymethyl or isopropoxyethyl.

Examples of possible three- to ten-membered mono- or bicyclic ring systems include carbocyclyls (such as cycloalkyls, cycloalkenyls), aryls, and five or six membered heterocyclyls or heteroaryls each containing from 1 to 4 heteroatoms each independently selected from the group consisting of nitrogen, oxygen and sulphur. The ring system may also comprise an alkylene linker, such as a methylene or an ethylene linker (such as benzyl).

Cycloalkyl groups preferably have from 3 to 6 ring carbon atoms, for example cyclopropyl (c-propyl (c-Pr)), cyclobutyl (c-butyl (c-Bu)), cyclopentyl (c-pentyl) and cyclohexyl (c-hexyl) and may be substituted or unsubstituted.

Heterocyclyls thus include, for example, tetrahydrofuranyl, tetrahydrothienyl and isoxazolinyl.

Heteroaryl thus includes, for example, benzofuranyl, benzimidazolyl, indolyl, isobenzofuranyl, furanyl, thienyl, thiazolyl, oxazolyl, isoxazolyl, isoquinolinyl, thiazolyl, pyrazolyl, isothiazolyl, pyridyl, pyridazinyl, pyrazinyl, pyrimidinyl, pyridonyl, quinolinyl, triazolyl, napthyridinyl and napthyridinonyl.

Aryl thus includes, for example phenyl and naphthyl and when incorporating a methylene linker, benzyl. The phenyl and benzyl groups may optionally be in a mono- or poly-substituted form, in which case the substituents may, as desired, be in the ortho-, meta- and/or para-position(s).

In a particular aspect of the present invention there is provided a compound of Formula (I), wherein A¹ is CH and A² is N.

In another aspect of the present invention there is provided a compound of Formula (I), wherein A¹ is N and A² is CH.

Particularly preferred is wherein A¹ and A² are both N.

In a preferred embodiment of the invention is a compound of Formula (I) wherein X¹ and X² are O.

In another aspect of the invention there is provided a compound of Formula (I) wherein A³ is N. In another aspect of the invention there is provided a compound of Formula (I) wherein A³ is CH. Compounds of Formula (I) wherein A³ is N are particularly preferred, as such compounds have been shown to exhibit surprising good activity compared to corresponding compounds wherein A³ is CH. Thus, in a particularly preferred embodiment of the present invention there is provided a compound of Formula (I) wherein A¹, A² and A³ are N, X¹ and X² are O.

In another preferred embodiment, R¹ is methyl, ethyl or propyl, preferably methyl.

In one aspect of the invention, R² is selected from the group consisting of hydrogen, C₁-C₆alkyl-, C₁-C₆haloalkyl-, C₁-C₃alkoxy-C₁-C₃alkyl-, C,-C₃ alkoxy-C₂-C₃alkoxy-C₁-C₃-alkyl-, C₁-C₃haloalkoxy-C₁-C₃-alkyl-, (C₁-C₃ alkylsulfonyl-C₁-C₃ alkylamino)-C₁-C₃ alkyl-, (C₁-C₃ alkylsulfonyl-C₃-C₄ cycloalkylamino)-C₁-C₃alkyl-, C₁-C₆alkylcarbonyl-C₁-C₃alkyl-, C₃-C₆cycloalkyl-C₂-C₆alkenyl-, C₂-C₆alkynyl-, C₂-C₆-alkenyl-, C₂-C₆-haloalkenyl-, cyano-C₁-C₆-alkyl-, arylcarbonyl-C₁-C₃-alkyl-, aryl-C₁-C₆alkyl-, aryloxy-C₁-C₆alkyl- (wherein said aryl groups may be optionally substituted with one or more substituents from the group consisting of halogen, C₁-C₃-alkoxy-, C₁-C₃-alkyl-, C₁-C₃ haloalkyl-), and a three- to ten-membered mono- or bicyclic ring system, which may be aromatic, saturated or partially saturated and can contain from 1 to 4 heteroatoms each independently selected from the group consisting of nitrogen, oxygen and sulphur the ring system being optionally substituted by one or more substituents selected from the group consisting of nitro, cyano, halogen, C₁-C₃alkyl-, C₁-C₃haloalkyl-, C₂-C₃alkenyl-, C₂-C₃alkynyl-, C₁-C₃ alkoxy-, C₁-C₃ haloalkoxy-, C₁-C₆alkyl-S(O)p- and C₁-C₆haloalkyl-S(O)p-.

In a preferred embodiment, R² is selected from the group consisting of C₁-C₆alkyl, C₁-C₃alkoxy-C₁-C₃alkyl-, C₁-C₃haloalkoxy-C₁-C₃-alkyl-, phenyl and a 5 or 6 membered heteroaromatic ring system containing from 1 to 4 heteroatoms each independently selected from the group consisting of nitrogen, oxygen and sulphur (e.g pyridyl, thienyl, furanyl, isoxazolyl, triazolyl); the phenyl or 5 or 6 heteroaromatic ring system being optionally substituted by one or more substituents selected from the group consisting of nitro, cyano, halogen, C₁-C₃alkyl-, C₁-C₃haloalkyl-, C₂-C₃alkenyl-, C₂-C₃alkynyl-, C₁-C₃alkoxy-, C₁-C₃ haloalkoxy-, C₁-C₆alkyl-S(O)p- and C₁-C₆haloalkyl-S(O)p-.

In another embodiment, R² is selected from the group consisting of C₁-C₆alkyl- (preferably methyl), C₁-C₃alkoxy-C₁-C₃alkyl-, C₁-C₆alkylcarbonyl-C₁-C₃alkyl-, benzyl-, pyridyl and phenyl-, the benzyl, pyridyl and phenyl groups being optionally substituted by one or more substituents selected from the group consisting of cyano, halogen (preferably fluorine or chlorine), C₁-C₃alkyl-, C₁-C₃haloalkyl-, C₂-C₃alkenyl-, C₁-C₃alkoxy- and C₁-C₃haloalkoxy-.

In another embodiment, R² is selected from the group consisting of C₁-C₆alkyl- (e.g n-butyl, cPr-(CH₂)-), C₁-C₃alkoxy-C₁-C₃alkyl- (e.g methoxyethyl-), C₁-C₆haloalkyl (e.g 4,4,4-trifluorobutyl, 3,3,3-trifluoropropyl), tetrahydropyranyl-(CH₂)-, benzyl- (optionally substituted by one or more substituents selected from the group consisting of nitro, halogen (e.g F, Cl), C₁-C₃haloalkyl- (e.g —CF₃) and C₁-C₃haloalkoxy- (e.g —OCHF₂) and phenyl- (optionally substituted by one or more substituents selected from the group consisting of C₁-C₆alkyl- (e.g methyl, i-propyl), C₁-C₃alkoxy-C₂-C₃alkoxy (e.g methoxyethoxy-), halogen (e.g F, Cl, Br), C₁-C₃haloalkyl- (e.g —CF₃), C₁-C₃haloalkoxy- (e.g —OCF₃) and C₁-C₃ alkoxy (e.g —OEt)).

In another embodiment R⁴ is methyl.

In another embodiment R⁵ is preferably hydrogen.

Compounds of Formula I may contain asymmetric centres and may be present as a single enantiomer, pairs of enantiomers in any proportion or, where more than one asymmetric centre are present, contain diastereoisomers in all possible ratios.

Typically one of the enantiomers has enhanced biological activity compared to the other possibilities.

Similarly, where there are disubstituted alkenes, these may be present in E or Z form or as mixtures of both in any proportion.

Furthermore, compounds of Formula (I) may be in equilibrium with alternative tautomeric forms. It should be appreciated that all tautomeric forms (single tautomer or mixtures thereof), racemic mixtures and single isomers are included within the scope of the present invention.

The present invention also includes agronomically acceptable salts that the compounds of Formula (I) may form with amines (for example ammonia, dimethylamine and triethylamine), alkali metal and alkaline earth metal bases or quaternary ammonium bases. Among the alkali metal and alkaline earth metal hydroxides, oxides, alkoxides and hydrogen carbonates and carbonates used as salt formers, emphasis is to be given to the hydroxides, alkoxides, oxides and carbonates of lithium, sodium, potassium, magnesium and calcium, but especially those of sodium, magnesium and calcium. The corresponding trimethylsulfonium salt may also be used.

The compounds of Formula (I) according to the invention can be used as herbicides by themselves, but they are generally formulated into herbicidal compositions using formulation adjuvants, such as carriers, solvents and surface-active agents (SFAs). Thus, the present invention further provides a herbicidal composition comprising a herbicidal compound according to any one of the previous claims and an agriculturally acceptable formulation adjuvant. The composition can be in the form of concentrates which are diluted prior to use, although ready-to-use compositions can also be made. The final dilution is usually made with water, but can be made instead of, or in addition to, water, with, for example, liquid fertilisers, micronutrients, biological organisms, oil or solvents.

The herbicidal compositions generally comprise from 0.1 to 99% by weight, especially from 0.1 to 95% by weight, compounds of Formula I and from 1 to 99.9% by weight of a formulation adjuvant which preferably includes from 0 to 25% by weight of a surface-active substance.

The compositions can be chosen from a number of formulation types, many of which are known from the Manual on Development and Use of FAO Specifications for Plant Protection Products, 5th Edition, 1999. These include dustable powders (DP), soluble powders (SP), water soluble granules (SG), water dispersible granules (WG), wettable powders (WP), granules (GR) (slow or fast release), soluble concentrates (SL), oil miscible liquids (OL), ultra low volume liquids (UL), emulsifiable concentrates (EC), dispersible concentrates (DC), emulsions (both oil in water (EW) and water in oil (EO)), micro-emulsions (ME), suspension concentrates (SC), aerosols, capsule suspensions (CS) and seed treatment formulations. The formulation type chosen in any instance will depend upon the particular purpose envisaged and the physical, chemical and biological properties of the compound of Formula (I).

Dustable powders (DP) may be prepared by mixing a compound of Formula (I) with one or more solid diluents (for example natural clays, kaolin, pyrophyllite, bentonite, alumina, montmorillonite, kieselguhr, chalk, diatomaceous earths, calcium phosphates, calcium and magnesium carbonates, sulphur, lime, flours, talc and other organic and inorganic solid carriers) and mechanically grinding the mixture to a fine powder.

Soluble powders (SP) may be prepared by mixing a compound of Formula (I) with one or more water-soluble inorganic salts (such as sodium bicarbonate, sodium carbonate or magnesium sulphate) or one or more water-soluble organic solids (such as a polysaccharide) and, optionally, one or more wetting agents, one or more dispersing agents or a mixture of said agents to improve water dispersibility/solubility. The mixture is then ground to a fine powder. Similar compositions may also be granulated to form water soluble granules (SG).

Wettable powders (WP) may be prepared by mixing a compound of Formula (I) with one or more solid diluents or carriers, one or more wetting agents and, preferably, one or more dispersing agents and, optionally, one or more suspending agents to facilitate the dispersion in liquids. The mixture is then ground to a fine powder. Similar compositions may also be granulated to form water dispersible granules (WG).

Granules (GR) may be formed either by granulating a mixture of a compound of Formula (I) and one or more powdered solid diluents or carriers, or from pre-formed blank granules by absorbing a compound of Formula (I) (or a solution thereof, in a suitable agent) in a porous granular material (such as pumice, attapulgite clays, fuller's earth, kieselguhr, diatomaceous earths or ground corn cobs) or by adsorbing a compound of Formula (I) (or a solution thereof, in a suitable agent) on to a hard core material (such as sands, silicates, mineral carbonates, sulphates or phosphates) and drying if necessary. Agents which are commonly used to aid absorption or adsorption include solvents (such as aliphatic and aromatic petroleum solvents, alcohols, ethers, ketones and esters) and sticking agents (such as polyvinyl acetates, polyvinyl alcohols, dextrins, sugars and vegetable oils). One or more other additives may also be included in granules (for example an emulsifying agent, wetting agent or dispersing agent).

Dispersible Concentrates (DC) may be prepared by dissolving a compound of

Formula (I) in water or an organic solvent, such as a ketone, alcohol or glycol ether. These solutions may contain a surface active agent (for example to improve water dilution or prevent crystallisation in a spray tank).

Emulsifiable concentrates (EC) or oil-in-water emulsions (EW) may be prepared by dissolving a compound of Formula (I) in an organic solvent (optionally containing one or more wetting agents, one or more emulsifying agents or a mixture of said agents). Suitable organic solvents for use in ECs include aromatic hydrocarbons (such as alkylbenzenes or alkylnaphthalenes, exemplified by SOLVESSO 100, SOLVESSO 150 and SOLVESSO 200; SOLVESSO is a Registered Trade Mark), ketones (such as cyclohexanone or methylcyclohexanone) and alcohols (such as benzyl alcohol, furfuryl alcohol or butanol), N-alkylpyrrolidones (such as N-methylpyrrolidone or N-octylpyrrolidone), dimethyl amides of fatty acids (such as C₈-C₁₀ fatty acid dimethylamide) and chlorinated hydrocarbons. An EC product may spontaneously emulsify on addition to water, to produce an emulsion with sufficient stability to allow spray application through appropriate equipment.

Preparation of an EW involves obtaining a compound of Formula (I) either as a liquid (if it is not a liquid at room temperature, it may be melted at a reasonable temperature, typically below 70° C.) or in solution (by dissolving it in an appropriate solvent) and then emulsifying the resultant liquid or solution into water containing one or more SFAs, under high shear, to produce an emulsion. Suitable solvents for use in EWs include vegetable oils, chlorinated hydrocarbons (such as chlorobenzenes), aromatic solvents (such as alkylbenzenes or alkylnaphthalenes) and other appropriate organic solvents which have a low solubility in water.

Microemulsions (ME) may be prepared by mixing water with a blend of one or more solvents with one or more SFAs, to produce spontaneously a thermodynamically stable isotropic liquid formulation. A compound of Formula (I) is present initially in either the water or the solvent/SFA blend. Suitable solvents for use in MEs include those hereinbefore described for use in in ECs or in EWs. An ME may be either an oil-in-water or a water-in-oil system (which system is present may be determined by conductivity measurements) and may be suitable for mixing water-soluble and oil-soluble pesticides in the same formulation. An ME is suitable for dilution into water, either remaining as a microemulsion or forming a conventional oil-in-water emulsion.

Suspension concentrates (SC) may comprise aqueous or non-aqueous suspensions of finely divided insoluble solid particles of a compound of Formula (I). SCs may be prepared by ball or bead milling the solid compound of Formula (I) in a suitable medium, optionally with one or more dispersing agents, to produce a fine particle suspension of the compound. One or more wetting agents may be included in the composition and a suspending agent may be included to reduce the rate at which the particles settle. Alternatively, a compound of Formula (I) may be dry milled and added to water, containing agents hereinbefore described, to produce the desired end product.

Aerosol formulations comprise a compound of Formula (I) and a suitable propellant (for example n-butane). A compound of Formula (I) may also be dissolved or dispersed in a suitable medium (for example water or a water miscible liquid, such as n-propanol) to provide compositions for use in non-pressurised, hand-actuated spray pumps.

Capsule suspensions (CS) may be prepared in a manner similar to the preparation of EW formulations but with an additional polymerisation stage such that an aqueous dispersion of oil droplets is obtained, in which each oil droplet is encapsulated by a polymeric shell and contains a compound of Formula (I) and, optionally, a carrier or diluent therefor. The polymeric shell may be produced by either an interfacial polycondensation reaction or by a coacervation procedure. The compositions may provide for controlled release of the compound of Formula (I) and they may be used for seed treatment. A compound of Formula (I) may also be formulated in a biodegradable polymeric matrix to provide a slow, controlled release of the compound.

The composition may include one or more additives to improve the biological performance of the composition, for example by improving wetting, retention or distribution on surfaces; resistance to rain on treated surfaces; or uptake or mobility of a compound of Formula (I). Such additives include surface active agents (SFAs), spray additives based on oils, for example certain mineral oils or natural plant oils (such as soy bean and rape seed oil), and blends of these with other bio-enhancing adjuvants (ingredients which may aid or modify the action of a compound of Formula (I).

Wetting agents, dispersing agents and emulsifying agents may be SFAs of the cationic, anionic, amphoteric or non-ionic type.

Suitable SFAs of the cationic type include quaternary ammonium compounds (for example cetyltrimethyl ammonium bromide), imidazolines and amine salts.

Suitable anionic SFAs include alkali metals salts of fatty acids, salts of aliphatic monoesters of sulphuric acid (for example sodium lauryl sulphate), salts of sulphonated aromatic compounds (for example sodium dodecylbenzenesulphonate, calcium dodecylbenzenesulphonate, butylnaphthalene sulphonate and mixtures of sodium di-isopropyl- and tri-isopropyl-naphthalene sulphonates), ether sulphates, alcohol ether sulphates (for example sodium laureth-3-sulphate), ether carboxylates (for example sodium laureth-3-carboxylate), phosphate esters (products from the reaction between one or more fatty alcohols and phosphoric acid (predominately mono-esters) or phosphorus pentoxide (predominately di-esters), for example the reaction between lauryl alcohol and tetraphosphoric acid; additionally these products may be ethoxylated), sulphosuccinamates, paraffin or olefine sulphonates, taurates and lignosulphonates.

Suitable SFAs of the amphoteric type include betaines, propionates and glycinates.

Suitable SFAs of the non-ionic type include condensation products of alkylene oxides, such as ethylene oxide, propylene oxide, butylene oxide or mixtures thereof, with fatty alcohols (such as oleyl alcohol or cetyl alcohol) or with alkylphenols (such as octylphenol, nonylphenol or octylcresol); partial esters derived from long chain fatty acids or hexitol anhydrides; condensation products of said partial esters with ethylene oxide; block polymers (comprising ethylene oxide and propylene oxide); alkanolamides; simple esters (for example fatty acid polyethylene glycol esters); amine oxides (for example lauryl dimethyl amine oxide); and lecithins

Suitable suspending agents include hydrophilic colloids (such as polysaccharides, polyvinylpyrrolidone or sodium carboxymethylcellulose) and swelling clays (such as bentonite or attapulgite).

The composition of the present may further comprise at least one additional pesticide. For example, the compounds according to the invention can also be used in combination with other herbicides or plant growth regulators. In a preferred embodiment the additional pesticide is a herbicide and/or herbicide safener. Examples of such mixtures are (in which ‘I’ represents a compound of Formula I). I+acetochlor, I+acifluorfen, I+acifluorfen-sodium, I+aclonifen, I+acrolein, I+alachlor, I+alloxydim, I+ametryn, I+amicarbazone, I+amidosulfuron, I+aminopyralid, I+amitrole, I+anilofos, I+asulam, I+atrazine, I+azafenidin, I+azimsulfuron, I+BCPC, I+beflubutamid, I+benazolin, I+bencarbazone, I+benfluralin, I+benfuresate, I+bensulfuron, I+bensulfuron-methyl, I+bensulide, I+bentazone, I+benzfendizone, I+benzobicyclon, I+benzofenap, I+bicyclopyrone, I+bifenox, I+bilanafos, I+bispyribac, I+bispyribac-sodium, I+borax, I+bromacil, I+bromobutide, I+bromoxynil, I+butachlor, I+butamifos, I+butralin, I+butroxydim, I+butylate, I+cacodylic acid, I+calcium chlorate, I+cafenstrole, I+carbetamide, I+carfentrazone, I+carfentrazone-ethyl, I+chlorflurenol, I+chlorflurenol-methyl, I+chloridazon, I+chlorimuron, I+chlorimuron-ethyl, I+chloroacetic acid, I+chlorotoluron, I+chlorpropham, I+chlorsulfuron, I+chlorthal, I+chlorthal-dimethyl, I+cinidon-ethyl, I+cinmethylin, I+cinosulfuron, I+cisanilide, I+clethodim, I+clodinafop, I+clodinafop-propargyl, I+clomazone, I+clomeprop, I+clopyralid, I+cloransulam, I+cloransulam-methyl, I+cyanazine, I+cycloate, I+cyclosulfamuron, I+cycloxydim, I+cyhalofop, I+cyhalofop-butyl, I+2,4-D, I+daimuron, I+dalapon, I+dazomet, I+2,4-DB, I+I+desmedipham, I+dicamba, I+dichlobenil, I+dichlorprop, I+dichlorprop-P, I+diclofop, I+diclofopmethyl, I+diclosulam, I+difenzoquat, I+difenzoquat metilsulfate, I+diflufenican, I+diflufenzopyr, I+dimefuron, I+dimepiperate, I+dimethachlor, I+dimethametryn, I+dimethenamid, I+dimethenamid-P, I+dimethipin, I+dimethylarsinic acid, I+dinitramine, I+dinoterb, I+diphenamid, I+dipropetryn, I+diquat, I+diquat dibromide, I+dithiopyr, I+diuron, I+endothal, I+EPTC, I+esprocarb, I+ethalfluralin, I+ethametsulfuron, I+ethametsulfuron-methyl, I+ethephon, I+ethofumesate, I+ethoxyfen, I+ethoxysulfuron, I+etobenzanid, I+fenoxaprop-P, I+fenoxaprop-P-ethyl, I+fenquinotrione, I+fentrazamide, I+ferrous sulfate, I+flamprop-M, I+flazasulfuron, I+florasulam, I+fluazifop, I+fluazifop-butyl, I+fluazifop-P, I+fluazifop-P-butyl, I+fluazolate, I+flucarbazone, I+flucarbazone-sodium, I+flucetosulfuron, I+fluchloralin, I+flufenacet, I+flufenpyr, I+flufenpyr-ethyl, I+flumetralin, I+flumetsulam, I+flumiclorac, I+flumiclorac-pentyl, I+flumioxazin, I+flumipropin, I+fluometuron, I+fluoroglycofen, I+fluoroglycofen-ethyl, I+fluoxaprop, I+flupoxam, I+flupropacil, I+flupropanate, I+flupyrsulfuron, I+flupyrsulfuron-methyl-sodium, I+flurenol, I+fluridone, I+flurochloridone, I+fluroxypyr, I+flurtamone, I+fluthiacet, I+fluthiacet-methyl, I+fomesafen, I+foramsulfuron, I+fosamine, I+glufosinate, I+glufosinate-ammonium, I+glyphosate, I+halauxifen, I+halosulfuron, I+halosulfuron-methyl, I+haloxyfop, I+haloxyfop-P, I+hexazinone, I+imazamethabenz, I+imazamethabenz-methyl, I+imazamox, I+imazapic, I+imazapyr, I+imazaquin, I+imazethapyr, I+imazosulfuron, I+indanofan, I+indaziflam, I+iodomethane, I+iodosulfuron, I+iodosulfuron-methyl-sodium, I+ioxynil, I+isoproturon, I+isouron, I+isoxaben, I+isoxachlortole, I+isoxaflutole, I+isoxapyrifop, I+karbutilate, I+lactofen, I+lenacil, I+linuron, I+mecoprop, I+mecoprop-P, I+mefenacet, I+mefluidide, I+mesosulfuron, I+mesosulfuronmethyl, I+mesotrione, I+metam, I+metamifop, I+metamitron, I+metazachlor, I+methabenzthiazuron, I+methazole, I+methylarsonic acid, I+methyldymron, I+methyl isothiocyanate, I+metolachlor, I+S-metolachlor, I+metosulam, I+metoxuron, I+metribuzin, I+metsulfuron, I+metsulfuron-methyl, I+molinate, I+monolinuron, I+naproanilide, I+napropamide, I+naptalam, I+neburon, I+nicosulfuron, I+n-methyl glyphosate, I+nonanoic acid, I+norflurazon, I+oleic acid (fatty acids), I+orbencarb, I+orthosulfamuron, I+oryzalin, I+oxadiargyl, I+oxadiazon, I+oxasulfuron, I+oxaziclomefone, I+oxyfluorfen, I+paraquat, I+paraquat dichloride, I+pebulate, I+pendimethalin, I+penoxsulam, I+pentachlorophenol, I+pentanochlor, I+pentoxazone, I+pethoxamid, I+phenmedipham, I+picloram, I+picolinafen, I+pinoxaden, I+piperophos, I+pretilachlor, I+primisulfuron, I+primisulfuron-methyl, I+prodiamine, I+profoxydim, I+prohexadione-calcium, I+prometon, I+prometryn, I+propachlor, I+propanil, I+propaquizafop, I+propazine, I+propham, I+propisochlor, I+propoxycarbazone, I+propoxycarbazone-sodium, I+propyzamide, I+prosulfocarb, I+prosulfuron, I+pyraclonil, I+pyraflufen, I+pyraflufen-ethyl, I+pyrasulfotole, I+pyrazolynate, I+pyrazosulfuron, I+pyrazosulfuron-ethyl, I+pyrazoxyfen, I+pyribenzoxim, I+pyributicarb, I+pyridafol, I+pyridate, I+pyriftalid, I+pyriminobac, I+pyriminobac-methyl, I+pyrimisulfan, I+pyrithiobac, I+pyrithiobac-sodium, I+pyroxasulfone, I+pyroxsulam, I+quinclorac, I+quinmerac, I+quinoclamine, I+quizalofop, I+quizalofop-P, I+rimsulfuron, I+saflufenacil, I+sethoxydim, I+siduron, I+simazine, I+simetryn, I+sodium chlorate, I+sulcotrione, I+sulfentrazone, I+sulfometuron, I+sulfometuron-methyl, I+sulfosate, I+sulfosulfuron, I+sulfuric acid, I+tebuthiuron, I+tefuryltrione, I+tembotrione, I+tepraloxydim, I+terbacil, I+terbumeton, I+terbuthylazine, I+terbutryn, I+thenylchlor, I+thiazopyr, I+thifensulfuron, I+thiencarbazone, I+thifensulfuron-methyl, I+thiobencarb, I+topramezone, I+tralkoxydim, I+tri-allate, I+triasulfuron, I+triaziflam, I+tribenuron, I+tribenuron-methyl, I+triclopyr, I+trietazine, I+trifloxysulfuron, I+trifloxysulfuron-sodium, I+trifluralin, I+triflusulfuron, I+triflusulfuron-methyl, I+trihydroxytriazine, I+trinexapac-ethyl, I+tritosulfuron, I+[3-[2-chloro-4-fluoro-5-(1-methyl-6-trifluoromethyl-2,4-dioxo-1,2,3,4-tetrahydropyrimidin-3-yl)phenoxy]-2-pyridyloxy]acetic acid ethyl ester (CAS RN 353292-31-6). The compounds of the present invention may also be combined with herbicidal compounds disclosed in WO06/024820 and/or WO07/096576.

The mixing partners of the compound of Formula I may also be in the form of esters or salts, as mentioned e.g. in The Pesticide Manual, Fifteenth Edition, British Crop Protection Council, 2009.

The compound of Formula I can also be used in mixtures with other agrochemicals such as fungicides, nematicides or insecticides, examples of which are given in The Pesticide Manual.

The mixing ratio of the compound of Formula I to the mixing partner is preferably from 1:100 to 1000:1.

The mixtures can advantageously be used in the above-mentioned formulations (in which case “active ingredient” relates to the respective mixture of compound of Formula I with the mixing partner).

The compounds of Formula I according to the invention can also be used in combination with one or more safeners. Likewise, mixtures of a compound of Formula I according to the invention with one or more further herbicides can also be used in combination with one or more safeners. The safeners can be AD 67 (MON 4660), benoxacor, cloquintocet-mexyl, cyprosulfamide (CAS RN 221667-31-8), dichlormid, fenchlorazole-ethyl, fenclorim, fluxofenim, furilazole and the corresponding R isomer, isoxadifen-ethyl, mefenpyr-diethyl, oxabetrinil, N-isopropyl-4-(2-methoxy-benzoylsulfamoyl)-benzamide (CAS RN 221668-34-4). Other possibilities include safener compounds disclosed in, for example, EP0365484 e.g N-(2-methoxybenzoyl)-4-[(methylaminocarbonyl)amino]benzenesulfonamide.

Particularly preferred are mixtures of a compound of Formula I with cyprosulfamide, isoxadifen-ethyl, cloquintocet-mexyl and/or N-(2-methoxybenzoyl)-4-[(methyl-aminocarbonyl)amino]benzenesulfonamide.

The safeners of the compound of Formula I may also be in the form of esters or salts, as mentioned e.g. in The Pesticide Manual, 15^{th} Edition (BCPC), 2009. The reference to cloquintocet-mexyl also applies to a lithium, sodium, potassium, calcium, magnesium, aluminium, iron, ammonium, quaternary ammonium, sulfonium or phosphonium salt thereof as disclosed in WO 02/34048, and the reference to fenchlorazole-ethyl also applies to fenchlorazole, etc.

Preferably the mixing ratio of compound of Formula I to safener is from 100:1 to 1:10, especially from 20:1 to 1:1.

The mixtures can advantageously be used in the above-mentioned formulations (in which case “active ingredient” relates to the respective mixture of compound of Formula I with the safener).

The present invention still further provides a method of selectively controlling weeds at a locus comprising crop plants and weeds, wherein the method comprises application to the locus of a weed controlling amount of a composition according to the present invention. ‘Controlling’ means killing, reducing or retarding growth or preventing or reducing germination. Generally the plants to be controlled are unwanted plants (weeds). ‘Locus’ means the area in which the plants are growing or will grow.

The rates of application of compounds of Formula I may vary within wide limits and depend on the nature of the soil, the method of application (pre- or post-emergence; seed dressing; application to the seed furrow; no tillage application etc.), the crop plant, the weed(s) to be controlled, the prevailing climatic conditions, and other factors governed by the method of application, the time of application and the target crop. The compounds of Formula I according to the invention are generally applied at a rate of from 10 to 2000 g/ha, especially from 50 to 1000 g/ha.

The application is generally made by spraying the composition, typically by tractor mounted sprayer for large areas, but other methods such as dusting (for powders), drip or drench can also be used.

Useful plants in which the composition according to the invention can be used include crops such as cereals, for example barley and wheat, cotton, oilseed rape, sunflower, maize, rice, soybeans, sugar beet, sugar cane and turf.

Crop plants can also include trees, such as fruit trees, palm trees, coconut trees or other nuts. Also included are vines such as grapes, fruit bushes, fruit plants and vegetables.

Crops are to be understood as also including those crops which have been rendered tolerant to herbicides or classes of herbicides (e.g. ALS-, GS-, EPSPS-, PPO-, ACCase- and HPPD-inhibitors) by conventional methods of breeding or by genetic engineering. An example of a crop that has been rendered tolerant to imidazolinones, e.g. imazamox, by conventional methods of breeding is Clearfield® summer rape (canola). Examples of crops that have been rendered tolerant to herbicides by genetic engineering methods include e.g. glyphosate- and glufosinate-resistant maize varieties commercially available under the trade names RoundupReady® and LibertyLink®.

In a preferred embodiment the crop plant is rendered tolerant to HPPD-inhibitors via genetic engineering. Methods of rending crop plants tolerant to HPPD-inhibitors are known, for example from WO0246387. Thus in an even more preferred embodiment the crop plant is transgenic in respect of a polynucleotide comprising a DNA sequence which encodes an HPPD-inhibitor resistant HPPD enzyme derived from a bacterium, more particularly from Pseudomonas fluorescens or Shewanella colwelliana, or from a plant, more particularly, derived from a monocot plant or, yet more particularly, from a barley, maize, wheat, rice, Brachiaria, Cenchrus, Lolium, Festuca, Setaria, Eleusine, Sorghum or Avena species. Several HPPD-tolerant soybean transgenic “events” are known, and include for example SYHTO4R (WO2012/082542), SYHT0H2 (WO2012/082548) and FG72.

Crops are also to be understood as being those which have been rendered resistant to harmful insects by genetic engineering methods, for example Bt maize (resistant to European corn borer), Bt cotton (resistant to cotton boll weevil) and also Bt potatoes (resistant to Colorado beetle). Examples of Bt maize are the Bt 176 maize hybrids of NK® (Syngenta Seeds). The Bt toxin is a protein that is formed naturally by Bacillus thuringiensis soil bacteria. Examples of toxins, or transgenic plants able to synthesise such toxins, are described in EP-A-451 878, EP-A-374 753, WO 93/07278, WO 95/34656, WO 03/052073 and EP-A-427 529. Examples of transgenic plants comprising one or more genes that code for an insecticidal resistance and express one or more toxins are KnockOut® (maize), Yield Gard® (maize), NuCOTIN33B® (cotton), Bollgard® (cotton), NewLeaf® (potatoes), NatureGard® and Protexcta®. Plant crops or seed material thereof can be both resistant to herbicides and, at the same time, resistant to insect feeding (“stacked” transgenic events). For example, seed can have the ability to express an insecticidal Cry3 protein while at the same time being tolerant to glyphosate.

Crops are also to be understood to include those which are obtained by conventional methods of breeding or genetic engineering and contain so-called output traits (e.g. improved storage stability, higher nutritional value and improved flavour).

Other useful plants include turf grass for example in golf-courses, lawns, parks and roadsides, or grown commercially for sod, and ornamental plants such as flowers or bushes.

The compositions can be used to control unwanted plants (collectively, ‘weeds’). The weeds to be controlled may be both monocotyledonous species, for example Agrostis, Alopecurus, Avena, Brachiaria, Bromus, Cenchrus, Cyperus, Digitaria, Echinochloa, Eleusine, Lolium, Monochoria, Rottboellia, Sagittaria, Scirpus, Setaria and Sorghum, and dicotyledonous species, for example Abutilon, Amaranthus, Ambrosia, Chenopodium, Chrysanthemum, Conyza, Galium, Ipomoea, Nasturtium, Sida, Sinapis, Solanum, Stellaria, Veronica, Viola and Xanthium. Weeds can also include plants which may be considered crop plants but which are growing outside a crop area (escapes), or which grow from seed left over from a previous planting of a different crop (volunteers). Such volunteers or escapes may be tolerant to certain other herbicides.

The present invention further provides a compound of Formula (II)

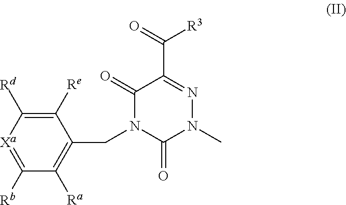

wherein

X^{a}=CR^{C} or N;

R^{a}, R^{b}, R^{c}, R^{d} and R^{e} are independently selected from the group consisting of nitro, cyano, halogen, C₁-C₃alkyl, C₁-C₃haloalkyl, C₂-C₃alkenyl, C₂-C₃alkynyl, C₁-C₃ alkoxy, C₁-C₃alkoxy-C₁-C₃alkyl-, C₁-C₃alkoxy-C₂-C₃alkoxy-, C₁-C₃alkoxy-C₂-C₃alkoxy-C₁-C₃-alkyl-, C₁-C₃ haloalkoxy, C₁-C₆alkyl-S(O)p- and C₁-C₆haloalkyl-S(O)p-;

R³ =hydroxyl or C₁-C₃ alkoxy; and p=0, 1 or 2.

The compounds of the present invention can be prepared according to the following schemes.

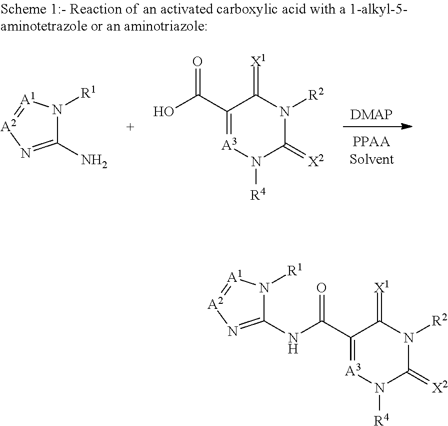

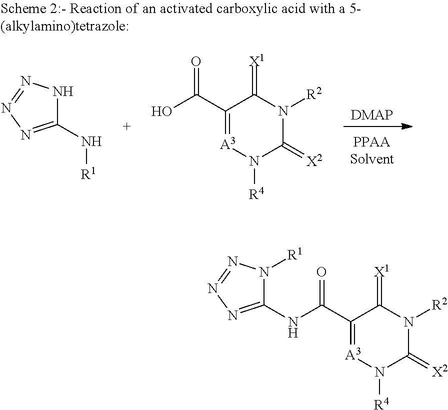

In each case, DMAP=4-(dimethylamino) pyridine, PPAA=1-propanephosphonic acid cyclic anhydride, and the solvent is a non-protic organic solvent such as ethyl acetate or dichloromethane.

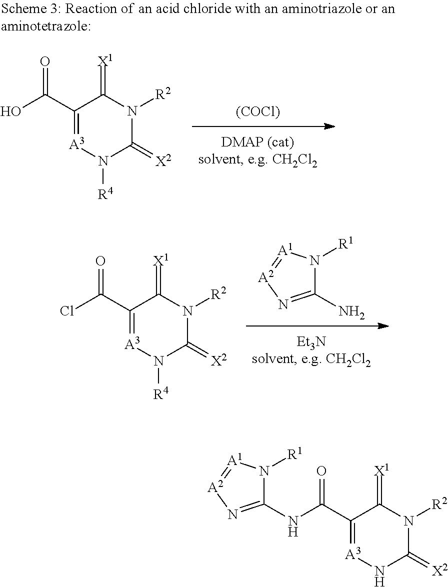

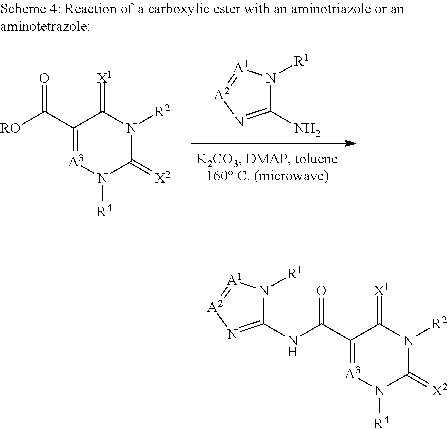

The carboxylic acids and esters can be prepared by known methods, or methods analogous to known methods. Examples of such methods are given below.

The carboxylic acids and esters may also be prepared by ring synthesis. In cases where the ring is a triazinedione, the ring can be constructed by the method shown in Scheme 5.

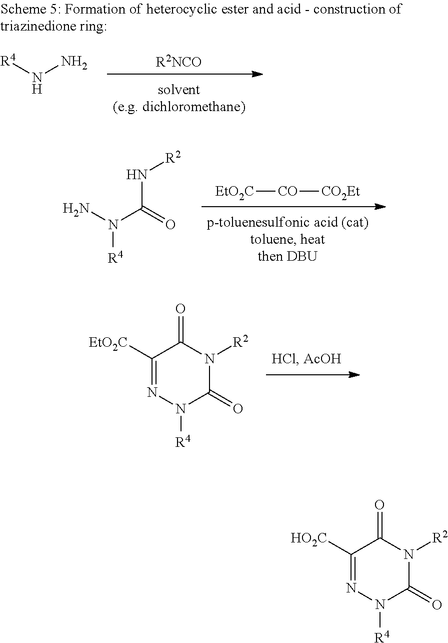

Wherein DBU is diazabicyclo[5.4.0]undec-7-ene.

In cases where the heterocyclic carboxylic acid or ester is a triazinedione and R⁴ is an alkyl group, the R⁴ group can be installed via a selective alkylation reaction as shown in Scheme 6.

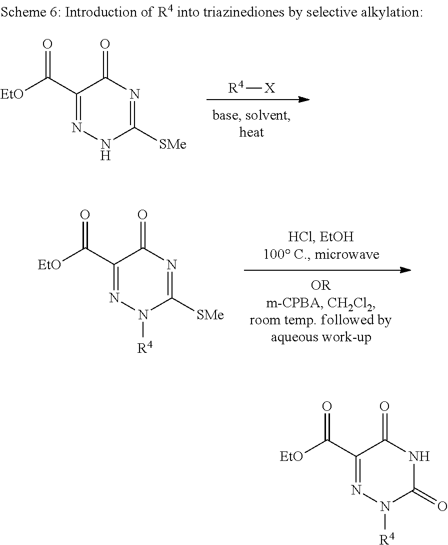

Wherein X is a halogen such as chlorine, bromine or iodine, the base is an inorganic base such as potassium carbonate, the solvent is a non-protic organic solvent such as toluene, and m-CPBA is meta-choroperbenzoic acid.

In cases where the heterocyclic carboxylic acid or ester is a pyrimidinedione and R⁴ is an alkyl group, the R⁴ group can be installed via a selective alkylation reaction as shown in Scheme 7.

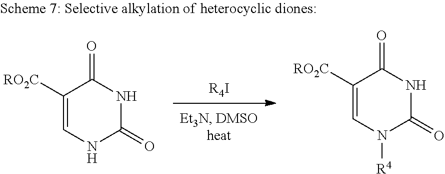

In cases where R² is alkyl, the carboxylic acid can be prepared by N-alkylation of the NH ester followed by de-esterification as shown in Scheme 8.

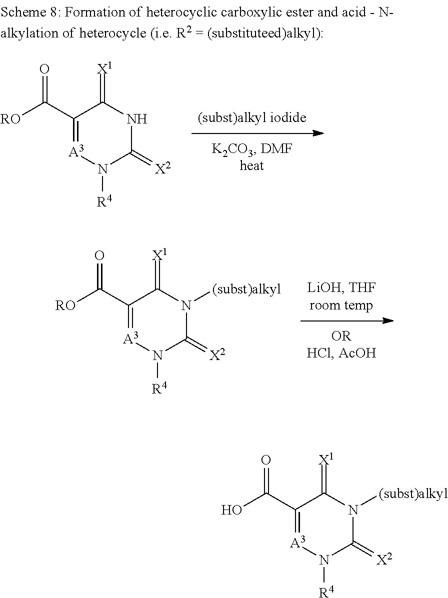

In cases where R² is aryl or heteroaryl, the carboxylic acid can be prepared by N-(hetero)arylation of the NH ester followed by de-esterification as shown in Scheme 9.

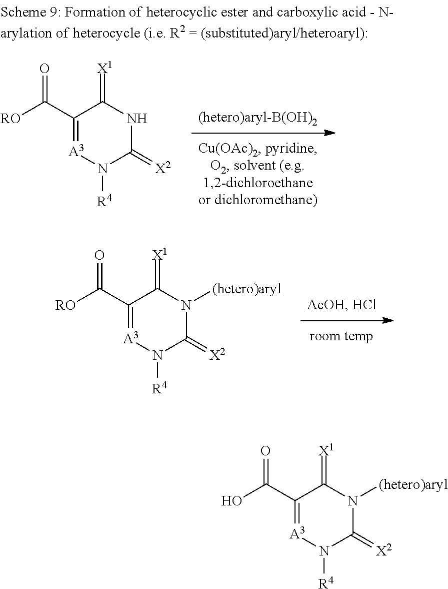

The aryl or heteroarylboronic acids are known or are readily available using established procedures.

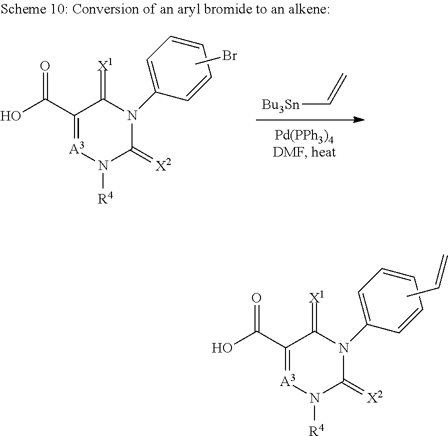

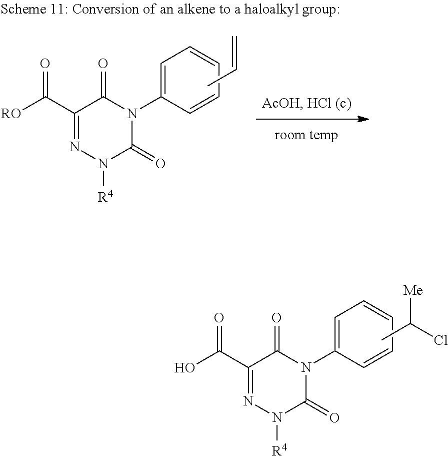

The following non-limiting examples provide specific synthesis methods for representative compounds of the present invention, as referred to in Tables 1 to 6 below.

### EXAMPLE P1

### Experimental Procedure for the Synthesis of Compounds 4.005 and 4.003.

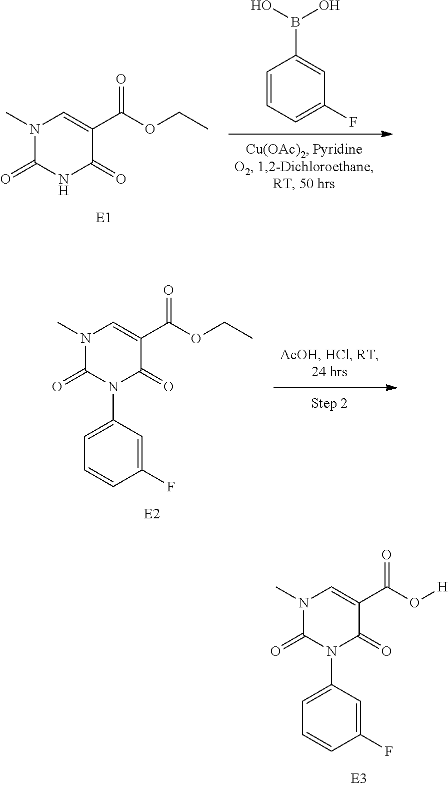 [SMILES: CCOC(=O)c1cn(C)c(=O)[nH]c1=O ; OB(O)c1cccc(F)c1 ; CCOC(=O)c1cn(C)c(=O)n(-c2cccc(F)c2)c1=O ; Cn1cc(C(=O)O)c(=O)n(-c2cccc(F)c2)c1=O]

STEP 1: A suspension of ester E1 (3.2 g, 16 mmol), copper II acetate (4 g, 20 mmol), in 1,2-dichloroethane (100 mL) containing pyridine (5.3 mL, 65 mmol) was vacuum filled with oxygen several times, then 3-fluorophenylboronic acid (3 g, 21.4 mmol) was added and stirred at room temperature for 24 hours with continuous bubbling of oxygen through the mixture. After 24 hours an extra amount of copper II acetate (2.5 g, 12.5 mmol) and 3-fluorophenylboronic acid (1.5 g, 10.7 mmol) were added and the reaction mixture was stirred at room temperature for another 24 hours with continuous bubbling of oxygen through the mixture. The reaction mixture was then diluted with water (100 mL) and extracted with ethyl acetate (300 mL×2). The combined organic extract was dried over sodium sulfate, filtered and concentrated under reduced pressure. The resulting crude product was purified by column chromatography; the desired product E2 was eluted with 2% methanol dichloromethane, and was obtained as a white solid (4.2 g).

STEP 2: Conc.HCl (25 mL) was added slowly at 0-5° C. to a solution of E2 (3.5 g, 11.975 mmoles) in acetic acid (25 mL), and the mixture was then stirred at room temperature for 24 hours. All volatiles were removed by concentrating under reduced pressure, and the residue was then triturated with ethyl acetate (100 mL) and stirred vigorously. The solid was filtered off, washed with diethyl ether (100 mL) and finally dried to obtain the pure desired product E3 as white solid (1.84 g).

Compounds 4.005 and 4.003 were then produced via coupling with the appropriate 1-alkyl-5-aminotetrazole.

### EXAMPLE P2

### Experimental Procedure for the Synthesis of Compound 1.001.

STEP 1.

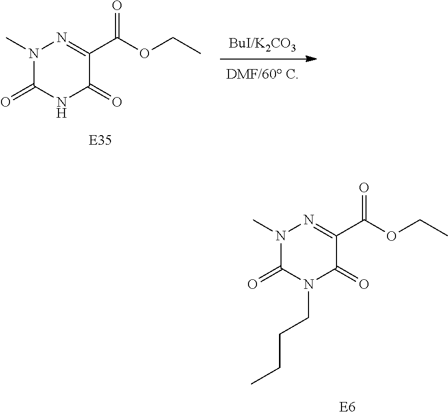 [SMILES: CCOC(=O)c1nn(C)c(=O)[nH]c1=O ; CCCCn1c(=O)c(C(=O)OCC)nn(C)c1=O]

To the solution of ester E35 (20 g, 100 mmol) in dimethyl formamide (DMF; 200 mL) under a nitrogen atmosphere was added potassium carbonate (41 g, 302 mmol) followed by dropwise addition of n-butyl iodide (28 g, 151 mmol) over a period of 15 min at room temperature. The reaction mixture was then warmed to 60° C. for 2hours and was then cooled, quenched with ice cold water (300 mL) and extracted with dichloromethane (500 mL×3). The combined dichloromethane extracts were dried over sodium sulfate, filtered and concentrated under reduced pressure. The crude material was purified by column chromatography; the desired product E6 was eluted with 20% ethyl acetate in hexane (18 g).

The ester E6 is then converted to the corresponding carboxylic acid and then coupled with the relevant 1-alkyl-5-aminotetrazole to provide compound 1.001.

### EXAMPLE P3

### Experimental Procedure for the Synthesis of Compound 1.002.

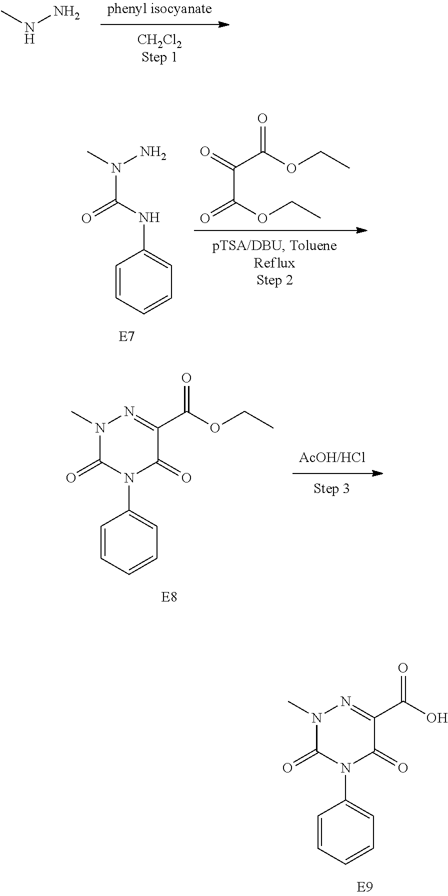

STEP 1: To a cold solution of methyl hydrazine (7.8 g , 168 mmoles) in dichloromethane (40 mL) on an ice-water bath was added dropwise a solution of phenyl isocyanate (20 g, 168 mmoles) in dichloromethane (40 mL) under a nitrogen atmosphere. The mixture was stirred for an hour at the same temperature, and then hexane (400 mL) was added and the resulting solid was filtered off, washed with hexane (100 mL) and dried under vacuum to afford compound E7 as white solid (22 g).

STEP 2: A mixture of compound E7 (20 g, 121 mmol), para-toluenesulphonic acid (pTSA; 3.1 g, 18 mmoles) and diethylketomalonate (15.6 g, 90 mmoles) in toluene (350 mL) was heated under reflux for 2 hours using a Dean Stark apparatus under a nitrogen atmosphere. The reaction mixture was then cooled to room temperature, DBU (6.82 g, 45 mmol) was added and the mixture was stirred at room temperature for another 2 hours. The mixture was then diluted with ethyl acetate (1.2 L) and washed with water (600 mL). The water layer was extracted with 600 mL ethyl acetate, and the combined ethyl acetate layers were dried over sodium sulfate, filtered, concentrated under reduced pressure and purified by column chromatography. The desired product E8 (13 g) was eluted with 16% ethyl acetate in hexane.

STEP 3: To a cold solution of E8 (10 g, 36.3 mmoles) in acetic acid (AcOH: 50 mL) on an ice-water bath was added conc. HCl (50 mL) dropwise, and the mixture was stirred at room temperature for 24 hours. All volatiles were removed from reaction mixture by concentrating under reduced pressure, and the residue was neutralized with saturated aqueous sodium carbonate and washed with ethyl acetate (300 mL). The aqueous layer was cooled in an ice-water bath and acidified with cold aqueous HCl solution (6 N) until a white precipitate appeared. After complete precipitation the solid was filtered off, washed with a small volume of ice-cold water and finally dried under reduced pressure to afford the pure acid E9 (6 g).

STEP 4:

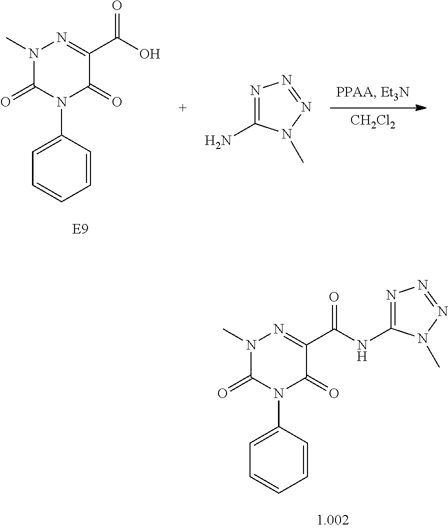

To a solution of the carboxylic acid E9 (250 mg, 1.10 mmol) and 5-amino-1-methyltetrazole (100 mg, 1.01 mmol) in HPLC grade dichloromethane (10 mL), dry triethylamine (0.7 mL, 4.04 mmol) was added dropwise at 0° C. and the reaction mixture was then stirred at room temperature for 10 min. 1-Propanephosphonic acid cyclic anhydride (PPAA; 1.8 mL, 3.03 mmol) was added, and the reaction mixture was allowed to stir at room temperature for a further 2 hours. The reaction mixture was diluted with 50 mL dichloromethane. The dichloromethane solution was washed with water (1×10 mL), brine (1×10 mL), dried over sodium sulfate and evaporated under reduced pressure to afford the crude product, which was purified on a CombiFlash Rf using ethyl acetate-hexane to afford the compound 1.002 as a white solid (160 mg).

### EXAMPLE P4

### Experimental Procedure for the Synthesis of Compound 4.004.

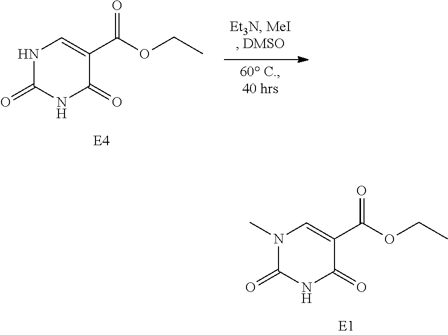

STEP 1. To a solution of compound E4 (11 g, 60 mmol) in dimethylsulfoxide (DMSO; 100 mL) was added triethylamine (55 mL), followed by slow addition of methyl iodide (55 mL) at 0-5° C. (exothermic). The reaction mixture was then brought to room temperature and finally stirred at 60° C. for 40 hours. All volatiles were removed from reaction mixture by concentrating under reduced pressure, and the residue was dissolved in ethyl acetate (1500 mL) and extracted with water (500 mL×5). The combined aqueous extracts were washed with ethyl acetate (250 mL) and then concentrated under reduced pressure. The crude product thus obtained was initially purified by column chromatography, and then pure product E1 was obtained as a white solid by recrystalization from a 50% solution of ethyl acetate in diethyl ether (7 g).

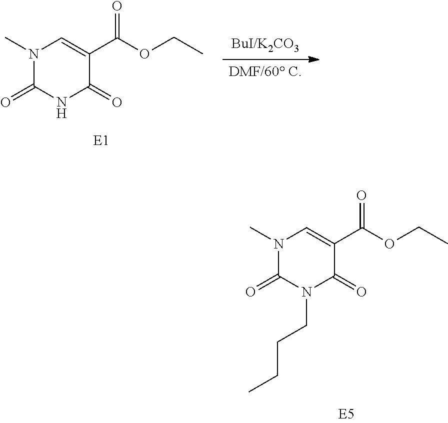 [SMILES: CCOC(=O)c1cn(C)c(=O)[nH]c1=O ; "DMF/60° C." ; CCCCn1c(=O)c(C(=O)OCC)cn(C)c1=O]

STEP 2: Ester E1 was alkylated with n-butyl iodide using a procedure analogous to that described in Example P2, step 1, to afford ester E5.

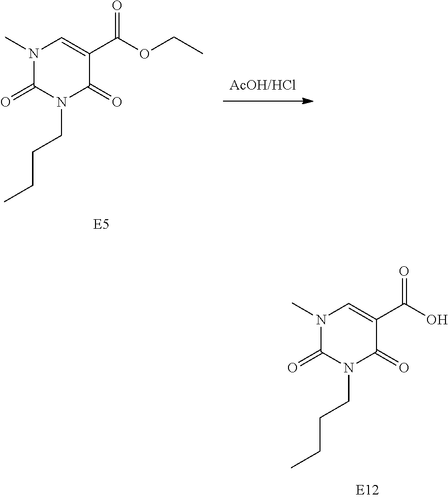 [SMILES: CCCCn1c(=O)c(C(=O)OCC)cn(C)c1=O ; CCCCn1c(=O)c(C(=O)O)cn(C)c1=O]

STEP 3: Ester E5 was hydrolysed to acid E12 using a procedure analogous to that described in Example P3, step 3.

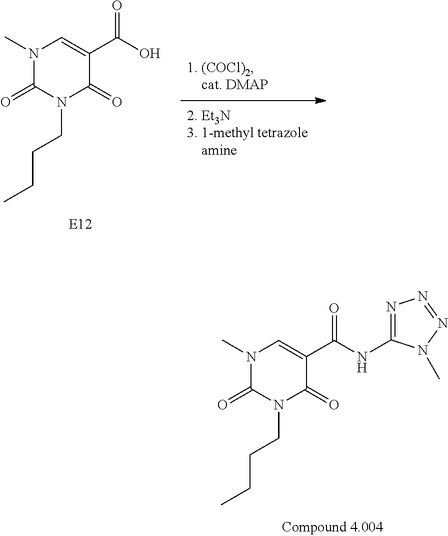 [SMILES: CCCCn1c(=O)c(C(=O)O)cn(C)c1=O ; CCCCn1c(=O)c(C(=O)Nc2nnnn2C)cn(C)c1=O]

STEP 4: To a suspension of the carboxylic acid E10 (100 mg, 0.443 mmol) in HPLC grade dichloromethane (5 mL), cat. 4-(dimethylamino)pyridine (DMAP) and oxalyl chloride (0.15 mL, 1.76 mmol) were added at 0° C. The reaction mixture was allowed to stir at room temperature for 2 hours, and was then concentrated under reduced pressure to obtain the crude acid chloride, which was used without further purification. The crude acid chloride was dissolved in HPLC grade dichloromethane (10 mL) and 5-amino-1-methyltetrazole (43 mg, 0.443 mmol) was added in one portion. After stirring for 10 min, dry triethylamine (0.25 mL. 1.76 mmol) was added dropwise at 0° C., and the reaction mixture was then allowed to stir for 16 hours at room temperature. The reaction mixture was then diluted with 30 mL dichloromethane, and the dichloromethane solution was washed with water (1×10 mL), brine (1×10 mL), dried over sodium sulfate and evaporated under reduced pressure to afford the crude product, which was purified on a CombiFlash Rf using ethyl acetate-hexane solvent to afford compound 4.004 as a white solid (70 mg).

### EXAMPLE P5

### Experimental Procedure for the Synthesis of Compound 1.003

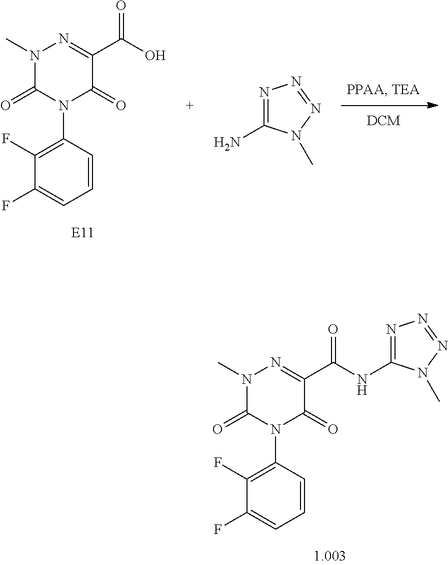

To a solution of the carboxylic acid E11 (200 mg, 0.706 mmol) and 5-amino-1-methyltetrazole (70 mg, 0.706 mmol) in HPLC grade dichloromethane (10 mL), dry triethylamine (TEA: 0.7 mL, 4.04 mmol) was added dropwise at 0° C. and the reaction mixture was then stirred at room temperature for 10 min. PPAA (1.8 mL, 2.12 mmol) was then added and the reaction mixture was stirred at room temperature for a further 16 hours. The reaction mixture was then diluted with 50 mL dichloromethane, and the dichloromethane solution was washed with water (1×10 mL), brine (1×10 mL), dried over sodium sulfate and evaporated under reduced pressure to afford the crude product, which was purified on a CombiFlash Rf using ethyl acetate-hexane to afford the desired compound 1.003 as a white solid (90 mg).

### EXAMPLE P6

### Experimental Procedure for the Synthesis of Compound 1.016

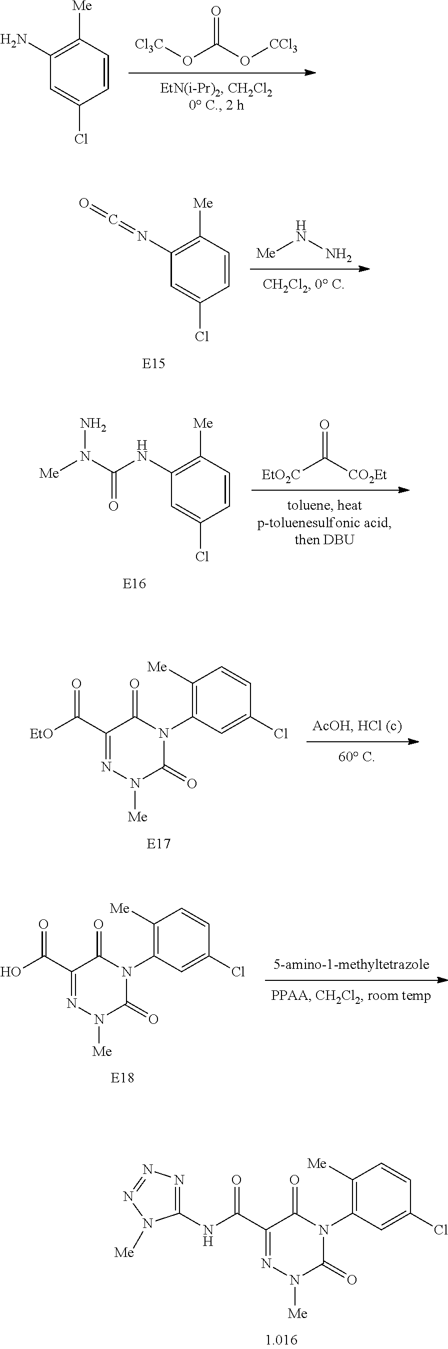

STEP 1: A stirred solution of 5-chloro-2-methyl-aniline (2 g, 14.12 mmol) in dichloromethane (40 mL) was treated dropwise with triphosgene (4.20 g, 14.2 mmol). A thick precipitate formed. The reaction mixture was then cooled to -5° C. (under nitrogen) and N,N-diisopropylethylamine (7.3 mL, 42 mmol) was added dropwise at such a rate to maintain the temperature of the mixture below 2° C. The precipitate dissolved as reaction proceeded. The reaction mixture was stirred at 0° C. for 2 hours and the solvent was then evaporated under reduced pressure to leave a yellow/brown solid. This solid was placed into a sinter and was slurried with diethyl ether (the solid was ground up to ensure full contact with the solvent). The mixture was filtered, and the solid was washed with more diethyl ether until the extracts were colourless. The ether filtrate was then evaporated under reduced pressure to leave the isocyanate E15 as a yellow/brown liquid, which was used in the next step without further purification.

STEP 2: A stirred solution of E15 (2.36 g, 14.10 mmol) in dichloromethane (30 mL) was flushed with nitrogen and cooled to 0° C. in an ice bath. Methylhydrazine (0.82 mL, 15.0 mmol) was added dropwise and the reaction mixture was stirred at 0° C. for 1 hour, and was then allowed to warm to room temperature. The reaction mixture was then stirred at room temperature overnight. The resulting solution was evaporated under reduced pressure to reduce the volume of solvent by approximately one half, then iso-hexane was added whereupon the product precipitated out. This was filtered off and washed with iso-hexane to afford E16 (2.873 g).

1Hnmr(CDCl₃): 8.60(s, 1H), 8.18(d, 1H), 7.04(d, 1H), 6.91(dd, 1H), 3.82(s, 2H), 3.27(s, 3H), 2.21(s, 3H)

STEP 3: A stirred solution of E16 (2.873 g, 13.45 mmol), diethyl 2-oxopropanedioate (2.1 mL, 14.0 mmol) and 4-toluenesulfonic acid (447 mg, 2.57 mmol) in toluene (50 mL) was treated with powdered molecular sieves (4A) and then heated under reflux under Dean and Stark conditions. Heating was continued for 2 hours, and then the mixture was cooled to room temperature. 1,8-Diazabicyclo[5.4.0]undec-7-ene (DBU; 0.32 mL, 2.1 mmol) was added, the reaction mixture was stirred at room temperature for 1 hour and was then left stand overnight. Water was added and the reaction mixture was extracted with ethyl acetate. The ethyl acetate extracts were filtered through Hi Flo to remove residual molecular sieves, and were then dried (MgSO4), filtered and evaporated under reduced pressure to leave an orange/brown viscous oil. This was purified twice by chromatography (CombiFlash Rf; 0-20% ethyl acetate/hexane) to afford Ell as a viscous yellow oil (944 mg).

1Hnmr(CDCl₃): 7.36(dd, 1H), 7.29(d, 1H), 7.12(d, 1H), 4.43(q, 2H), 3.79(s, 3H), 2.11(s, 3H), 1.41(t, 3H)

STEP 4: Hydrochloric acid (conc) (1 mL) was added dropwise to a stirred solution of E11 (944 mg, 2.916 mmol) in acetic acid (5 mL) . The reaction mixture was warmed to 60° C. for 2 hours and was then allowed to cool to room temperature. The mixture was evaporated to dryness under reduced pressure (toluene was added to azeotrope out the final traces of water) to leave E18 as a white foam (880 mg).

1Hnmr(CDCl₃): 7.44(dd, 1H), 7.38(d, 1H), 7.18(d, 1H), 3.91(s, 3H), 2.14(s, 3H)

STEP 5: A solution of E18 (880 mg, 2.976 mmol) and 5-amino-1-methyltetrazole (297 mg, 2.997 mmol) in dichloromethane (20 mL) were stirred together with 4-dimethylaminopyridine (DMAP) (727 mg, 5.891 mmol) for 3 hours. A solution of 1-propanephosphonic acid cyclic anhydride (PPAA) in ethyl acetate (50 mass %; 3.8 mL, 6.0 mmol) was then added, and the reaction mixture was allowed to stir at room temperature overnight. Water was added and the mixture was stirred vigorously. The organic layer was separated, evaporated under reduced pressure and separated by chromatography (CombiFlash RE 0-5% methanol in dichloromethane) to afford Compound 1.016 as a white solid (876 mg).

### EXAMPLE P7

### Experimental Procedure for the Synthesis of Compound 1.018

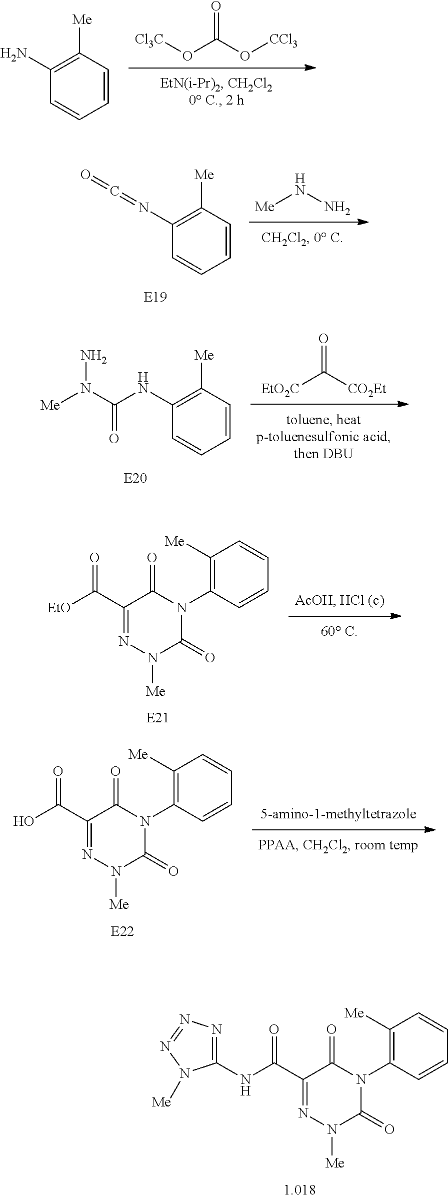

STEP 1: A stirred solution of 2-methyl-aniline (1 g, 9.332 mmol) in dichloromethane (20 mL) was treated dropwise with triphosgene (2.78 g, 9.37 mmol). A thick precipitate formed. The reaction mixture was then cooled to −5° C. (under nitrogen) and N,N-diisopropylethylamine (4.87 mL, 28.0 mmol) was added dropwise at such a rate to maintain the temperature of the mixture below 2° C. The precipitate dissolved as reaction proceeded. The reaction mixture was stirred at 0° C. for 2 hours and the solvent was then evaporated under reduced pressure to leave a yellow/brown solid.

This solid was placed into a sinter and was slurried with diethyl ether (the solid was ground up to ensure full contact with the solvent). The mixture was filtered, and the solid was washed with more diethyl ether until the extracts were colourless. The ether filtrate was then evaporated under reduced pressure to leave the isocyanate E19 as a yellow liquid, which was used in the next step without further purification.

STEP 2: A stirred solution of E19 (1.243 g, 9.335 mmol) in dichloromethane (25 mL) was flushed with nitrogen and cooled to 0° C. in an ice bath. Methylhydrazine (0.54 mL, 10.0 mmol) was then added dropwise and the reaction mixture was stirred at 0° C. for 1 hour, and was then allowed to warm to room temperature. The reaction mixture was then stirred at room temperature overnight. The resulting solution was evaporated under reduced pressure to remove the solvent, and the residue was triturated with hexane containing a little diethyl ether to afford E20 as a solid (1.325 g).

1Hnmr(CDCl₃): 8.55(s, 1H), 8.01(d, 1H), 7.19(t, 1H), 7.13(d, 1H), 6.94(t, 1H), 3.24(s, 3H), 2.25(s, 3H)

STEP 3: A strirred solution of E20 (500 mg, 2.79 mmol)), diethyl 2-oxopropanedioate (0.43 mL, 2.8 mmol) and 4-toluenesulfonic acid (96 mg, 0.552 mmol) in toluene (20 mL) was treated with powdered molecular sieves (4A) and then heated under reflux under Dean and Stark conditions. Heating was continued for 2 hours, and then the mixture was cooled to room temperature. DBU (1,8-diazabicyclo[5.4.0]undec-7-ene (0.07 mL, 0.5 mmol) was added, the reaction mixture was stirred at room temperature for 1 hour and was then left stand overnight. Water was added and the reaction mixture was extracted with ethyl acetate. The ethyl acetate extracts were filtered through Hi Flo to remove residual molecular sieves, and were then dried (MgSO4), filtered and evaporated under reduced pressure to leave an orange/brown viscous oil. This was purified by chromatography (CombiFlash Rf; 0-20% ethyl acetate/hexane) to afford E21 as a white solid (430 mg).

1Hnmr(CDCl₃): 7.40-7.29(m, 3H), 7.09(d, 1H), 4.43(q, 2H), 3.79(s, 3H), 2.14(s, 3H), 1.40(t, 3H)

STEP 4: Hydrochloric acid (conc) (1 mL) was added dropwise to a stirred solution of E21 (430 mg, 1.486 mmol) in acetic acid (4 mL). The reaction mixture was warmed to 60° C. for 1 hour and was then allowed to cool to room temperature and left to stand overnight. The mixture was evaporated to dryness under reduced pressure (toluene was added to azeotrope out the final traces of water) to leave E22 as a white foam (400 mg).

STEP 5: A solution of E22 (400 mg, 1.5312 mmol) and 5-amino-1-methyltetrazole (149 mg, 1.504 mmol) in dichloromethane (5 mL) were stirred together with DMAP (374 mg, 3.031 mmol) for 3 hours. A solution of 1-propanephosphonic acid cyclic anhydride (PPAA) in ethyl acetate (50 mass %; 1.97 mL, 3.1 mmol) was then added, and the reaction mixture was allowed to stir at room temperature overnight. Water was added and the mixture was stirred vigorously. The organic layer was separated, evaporated under reduced pressure and separated by chromatography (CombiFlash Rf. 0-5% methanol in dichloromethane) to afford Compound 1.018 as a white solid (121 mg).

### EXAMPLE P8

### Experimental Procedure for the Synthesis of Compound 1.020

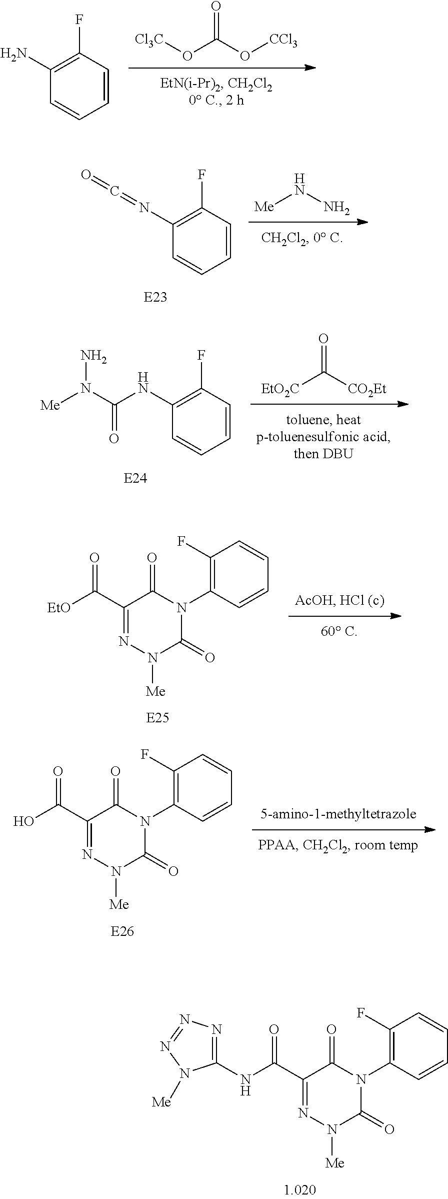

STEP 1: A stirred solution of 2-fluoroaniline (2 g, 17.20 mmol) in dichloromethane (50 mL) was treated dropwise with triphosgene (5.35 g, 18.0 mmol). A thick precipitate formed. The reaction mixture was then cooled to -5° C. (under nitrogen) and N,N-diisopropylethylamine (9.4 mL, 54 mmol) was added dropwise at such a rate to maintain the temperature of the mixture below 2° C. The precipitate dissolved as reaction proceeded. The reaction mixture was stirred at 0° C. for 1.5 hours and the solvent was then evaporated under reduced pressure to leave a yellow/brown solid. This solid was placed into a sinter and was slurried with diethyl ether (the solid was ground up to ensure full contact with the solvent). The mixture was filtered, and the solid was washed with more diethyl ether until the extracts were colourless. The ether filtrate was then evaporated under reduced pressure to leave the isocyanate E23 as a yellow liquid, which was used in the next step without further purification.

STEP 2: A stirred solution of E23 (2.47 g, 18.0 mmol) in dichloromethane (40 mL) was flushed with nitrogen and cooled to 0° C. in an ice bath. Methylhydrazine (1.05 mL, 19.7 mmol) was then added dropwise and the reaction mixture was stirred at 0° C. for 1 hour, and was then allowed to warm to room temperature. The reaction mixture was then stirred at room temperature overnight. The resulting solution was evaporated under reduced pressure to remove the solvent, and the residue was separated by chromatography (CombiFlash Rf; 0-2% methanol in dichloromethane, then up to 5%, then up to 50%) to afford E24 as a solid (547 mg). MS: m/e 182 (M-H)

STEP 3: A strirred solution of E24 (547 mg, 2.986 mmol), diethyl 2-oxopropanedioate (0.46 mL, 3.0 mmol) and 4-toluenesulfonic acid (103 mg, 0.592 mmol) in toluene (20 mL) was treated with powdered molecular sieves (4A) and then heated under reflux under Dean and Stark conditions. Heating was continued for 2 hours, and then the mixture was cooled to room temperature. DBU (1,8-diazabicyclo[5.4.0]undec-7-ene (0.09 mL, 0.6 mmol) was added, the reaction mixture was stirred at room temperature for 1 hour and was then left stand overnight. Water was added and the reaction mixture was extracted with ethyl acetate. The ethyl acetate extracts were filtered through Hi Flo to remove residual molecular sieves, and were then dried (MgSO₄), filtered and evaporated under reduced pressure to leave an orange/brown viscous oil. This was purified by chromatography (CombiFlash Rf; 0-15% ethyl acetate/hexane) to afford E25 as a white solid (200 mg).

1Hnmr(CDCl3): 7.52-7.46(m, 1H), 7.30-7.23(m, 3H), 4.44(q, 2H), 3.79(s, 3H), 1.40(t, 3H)

STEP 4: Hydrochloric acid (conc) (1 mL) was added dropwise to a stirred solution of E25 (200 mg, 0.682 mmol) in acetic acid (4 mL). The reaction mixture was warmed to 50° C. for 6 hours and was then allowed to cool to room temperature and left to stand overnight. The mixture was then heated to 50° C. for a further 2 hours, then allowed to cool. The mixture was evaporated to dryness under reduced pressure (toluene was added to azeotrope out the final traces of water) to leave E26 as a white foam (179 mg).

1Hnmr(CDCl₃): 7.52(m, 1H), 7.35-7.10(m, 3H), 3.94(s, 3H)

STEP 5: A solution of E26 (179 mg, 0.675 mmol) and 5-amino-1-methyltetrazole (67 mg, 0.676 mmol) in dichloromethane (5 mL) were stirred together with DMAP (165 mg, 1.337 mmol) for 3 hours. A solution of 1-propanephosphonic acid cyclic anhydride (PPAA) in ethyl acetate (50 mass %; 0.90 mL, 1.0 mmol) was then added, and the reaction mixture was stirred for 1 hour, then left to stand overnight. Water was added and the mixture was stirred vigorously. The organic layer was separated, evaporated under reduced pressure and separated by chromatography (CombiFlash Rf. 0-5% methanol in dichloromethane) to afford compound 1.020 as a pale yellow solid (115 mg).

### EXAMPLE P9

### Experimental Procedure for the Synthesis of Compound 1.021

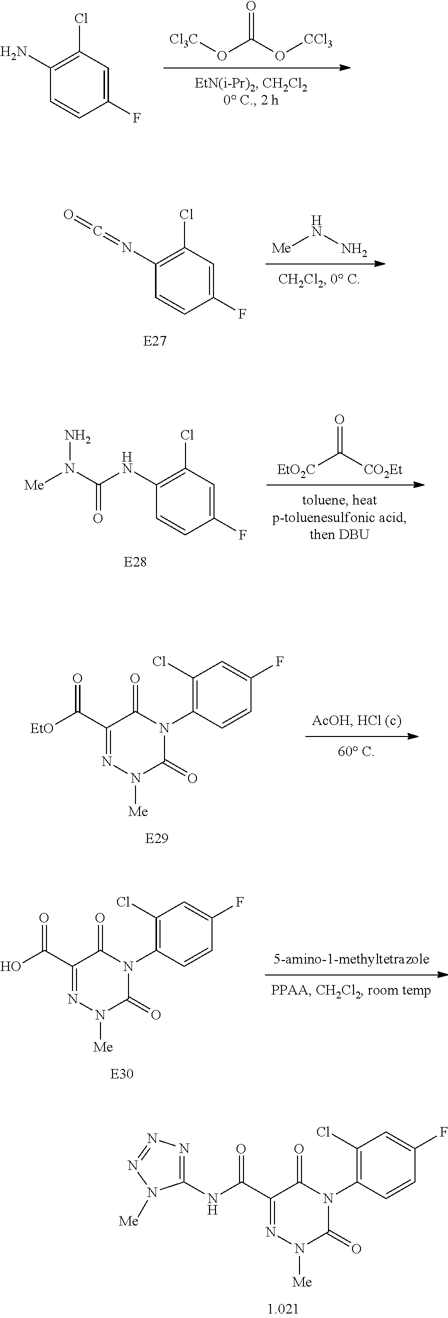

STEP 1: A stirred solution of 2-chloro-4-fluoroaniline (1.0 g, 6.870 mmol) in dichloromethane (20 mL) was treated dropwise with triphosgene (680 mg, 2.292 mmol). A thick precipitate formed. The reaction mixture was then cooled to -5° C. (under nitrogen) and N,N-diisopropylethylamine (1.2 mL,6.9 mmol) was added dropwise at such a rate to maintain the temperature of the mixture below 0° C. The precipitate dissolved as reaction proceeded, though some was left after the addition.

A second batch of triphsogene (680 mg, 2.292 mmol) was added, and 10 mins later a second batch of N,N-diisopropylethylamine (1.2 mL,6.9 mmol) was added dropwise at such a rate to maintain the temperature of the mixture below 0° C. The reaction mixture was stirred at 0° C. for 1.5 hours and the solvent was then evaporated under reduced pressure to leave a yellow/brown solid. This solid was placed into a sinter and was slurried with diethyl ether (the solid was ground up to ensure full contact with the solvent). The mixture was filtered, and the solid was washed with more diethyl ether until the extracts were colourless. The ether filtrate was then evaporated under reduced pressure to leave the isocyanate E27 as a red/brown liquid, which was used in the next step without further purification.

STEP 2: A stirred solution of E27 (1.17 g, 6.82 mmol) in dichloromethane (25 mL) was flushed with nitrogen and cooled to 0° C. in an ice bath. Methylhydrazine (0.40 mL, 7.50 mmol) was then added dropwise and the reaction mixture was stirred at 0° C. for 1 hour, and was then allowed to warm to room temperature. The reaction mixture was then left to stand at room temperature overnight. The resulting solution was evaporated under reduced pressure to reduce the volume of solvent by approximately one half, then iso-hexane was added whereupon the product precipitated out. This was filtered off and washed with iso-hexane to afford E28 (1.138 g).

1Hnmr(CDCl₃): 9.12(broad s, 1H), 8.28(dd, 1H), 7.09(dd, 1H), 6.96(dt, 1H), 3.84(broad s, 2H), 3.25(s, 3H)

STEP 3: A strirred solution of E28 (1.138 g, 5.229 mmol), diethyl 2-oxopropanedioate (0.80 mL, 5.0 mmol) and 4-toluenesulfonic acid (179 mg, 1.0291 mmol) in toluene (50 mL) was treated with powdered molecular sieves (4A) and then heated under reflux under Dean and Stark conditions. Heating was continued for 2 hours, and then the mixture was cooled to room temperature. DBU (1,8-diazabicyclo[5.4.0]undec-7-ene (0.15 mL, 1.0 mmol) was added, the reaction mixture was stirred at room temperature for 1 hour and was then left stand overnight. LCMS analysis indicated that the reaction had not proceeded to completion, so a further sample of DBU (0.8 mL) was added and the reaction mixture was stirred for a further 2 hours. Ethyl acetate was added and the mixture filtered through Hi Flo to remove the molecular sieves. The filtrate was washed with water and evaporated under reduced pressure (toluene was added to azeotrope out the final traces of water) to leave a red/orange viscous oil. This was purified by chromatography (CombiFlash Rf; 0-10% dichloromethane//methanol) to afford E29 as a viscous yellow oil (463 mg).

1Hnmr(CDCl₃): 7.34(dd, 1H), 7.26(dd, 1H), 7.15(ddd), 4.45(q, 2H), 3.80(s, 3H), 1.41(t, 3H)

STEP 4: Hydrochloric acid (conc) (1 mL) was added dropwise to a stirred solution of E29 (463mg, 1.413 mmol) in acetic acid (4 mL). The reaction mixture was warmed to 50° C. for 6 hours and was then allowed to cool to room temperature and left to stand overnight. The mixture was then heated to 50° C. for a further 2 hours, then allowed to cool. The mixture was evaporated to dryness under reduced pressure (toluene was added to azeotrope out the final traces of water) to leave E30 as a pale brown solid (381 mg).

1Hnmr(CDCl₃): 7.40(m, 1H), 7.30-7.10 (m, 3H), 3.95 (s, 3H)

STEP 5: A solution of E30 (381 mg, 1.272 mmol) and 5-amino-1-methyltetrazole (129 mg, 1.30 mmol) in dichloromethane (20 mL) were stirred together with DMAP (310 mg, 2.512 mmol) for 3 hours. A solution of 1-propanphosphonic acid cyclic anhydride (PPAA) in ethyl acetate (50 mass %; 1.59 mL, 2.50 mmol) was then added, and the reaction mixture was stirred for 1 hour, then left to stand overnight. Water was added and the mixture was stirred vigorously. The organic layer was separated, evaporated under reduced pressure and separated by chromatography (CombiFlash Rf. 0-5% methanol in dichloromethane) to afford compound 1.021 as a pale yellow solid (348 mg).

### EXAMPLE P10

### Experimental Procedure for the Synthesis of Compound 1.015

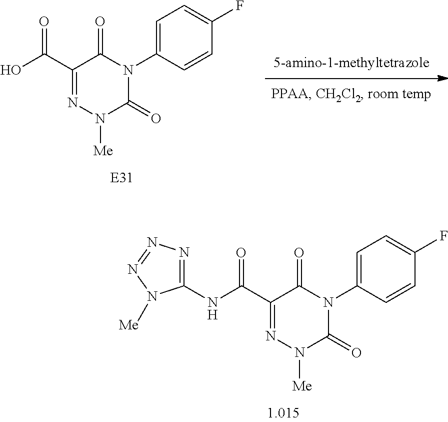 [SMILES: Cn1nc(C(=O)O)c(=O)n(-c2ccc(F)cc2)c1=O ; Cn1nnnc1NC(=O)c1nn(C)c(=O)n(-c2ccc(F)cc2)c1=O]

STEP 1: A solution of E31 (194 mg, 0.732 mmol) and 5-amino-1-methyltetrazole (72 mg, 0.727 mmol) in dichloromethane (20 mL) were stirred together with DMAP (179 mg, 1.451 mmol) for 3 hours. A solution of 1-propanephosphonic acid cyclic anhydride (PPAA) in ethyl acetate (50 mass %; 0.95 mL, 1.50 mmol) was then added, and the reaction mixture was stirred for 1 hour, then left to stand overnight. Water was added and the mixture was stirred vigorously. The organic layer was separated, evaporated under reduced pressure and separated by chromatography (CombiFlash Rf. 0-5% methanol in dichloromethane) to afford Compound 1.015 as a white solid (196 mg).

### EXAMPLE P11

### Experimental Procedure for the Synthesis of Compound 1.023

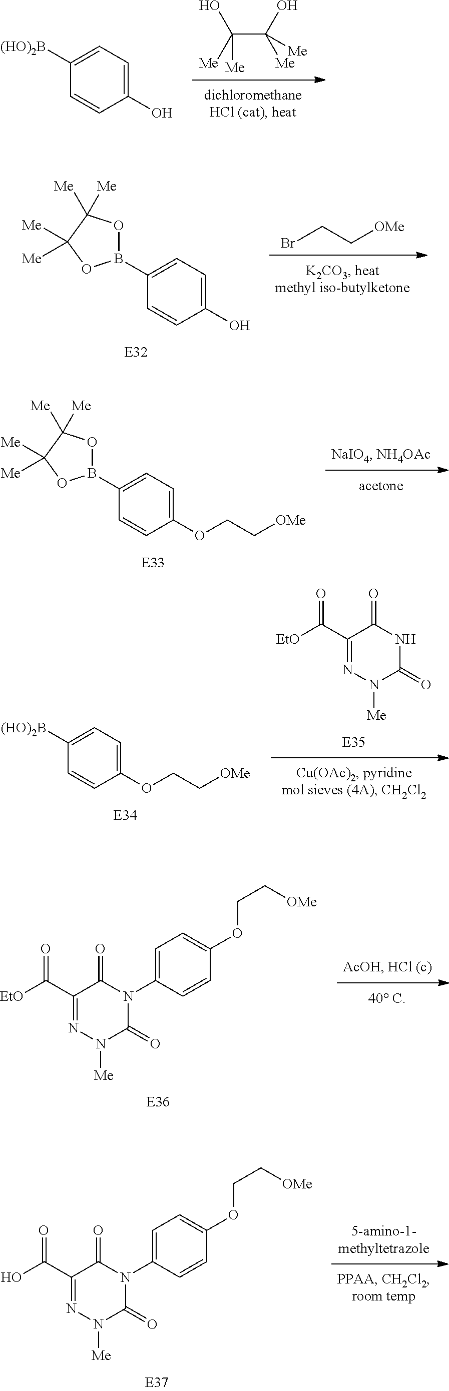

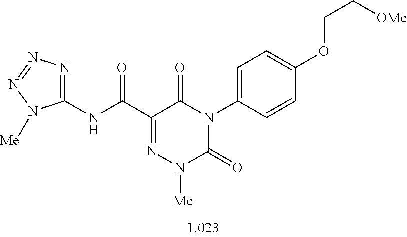 [SMILES: COCCOc1ccc(-n2c(=O)c(C(=O)Nc3nnnn3C)nn(C)c2=O)cc1]

STEP 1: A stirred solution of (4-hydroxyphenyl)boronic acid (2.0 g, 14.500 mmol) in dichloromethane (70 mL) was treated with 2,3-dimethylbutane-2,3-diol (1.7136 g, 14.500 mmol). The reaction mixture heated under reflux for several hours, and a drop of conc hydrochloric acid was added part-way through the procedure to catalyse the reaction. The reaction mixture was then cooled to room temperature and allowed to stand overnight. The reaction mixture was then filtered to remove a trace of insoluble material, and the filtrate was then evaporated under reduced pressure to afford E32 as a white solid (3.175 g)

1Hnmr(CDCl₃): 7.72 (2H, d), 6.83 (2H, d), 4.96 (1H, s, OH), 1.34 (12H, s)

STEP 2: A stirred solution of E32 (3.175 g, 14.43 mmol) in methyl isobutylketone (MIBK) (100 mL) was treated with (2.225 g, 15.87 mmol) and the mixture was stirred for 5 min at room temperature before the dropwise addition of 1-bromo-2-methoxyethane (3.108 g, 22.36 mmol). The resulting suspension was heated under reflux, with stirring, for 21 hours. The mixture was then cooled to room temperature and filtered. The filtrate was evaporated under reduced pressure to give 4.3 g of a brown oil. This was purified by chromatography (CombiFlash RE iso-hexane/ethyl acetate, 0% to 20%) to afford E33 as an almost colourless oil (3.471 g).

1Hnmr(CDCl₃): 7.75 (2H, d), 6.93 (2H, d), 4.16 (2H, m), 3.77 (2H, m), 3.46 (3H, s), 1.34 (12H, s)

STEP 3: A stirred solution of E33 (3.47 g, 12.50 mmol) and acetone (75 mL) was treated with a solution of ammonium acetate (4 equiv., 3.85 g, 49.9 mmol) in water (75 mL). Sodium periodate (4 equiv., 10.7 g, 49.9 mmol) was then added portionwise. The yellow/white suspension was vigourously stirred at room temperature for 36 hours. The mixture was then filtered, and the insoluble material was washed with ethyl acetate. The combined filtrates were evaporated under reduced pressure and the residue was extracted several times with ethyl acetate. The combined extracts were dried (MgSO₄) and evaporated under reduced pressure to afford the crude product as a white solid. This was purified by chromatography (CombiFlash RE iso-hexane/EtOAc, 0% to 70%) to afford E34 as a white solid (875 mg).

1Hnmr(CDCl₃): 8.14 (2H, d), 7.03(2H, d), 4.20 (2H, t), 3.79 (2H, t), 3.48 (3H, s)

STEP 4: A stirred solution of E34 (540 mg, 2.755 mmol) and E35 (400 mg, 2.008 mmol) in dichloromethane (40 mL) was treated with powdered 4A molecular sieves (100 mg), pyridine (0.635466 g, 8.0335 mmol) and copper (II) acetate (0.72956 g, 4.0167 mmol). The reaction mixture was stirred at room temperature for 22 hours. Ethyl acetate was then added and the mixture was filtered through Hi Flo (the copper salts were rinsed with more ethyl acetate). The green filtrate was evaporated under reduced pressure to leave a green solid residue. This was purified by chromatography (CombiFlash RE iso-hexane/EtOAc, 0% to 100%) to afford E36 as a white solid (506 mg).

1Hnmr(CDCl₃): 7.13 (2H, m), 7.03 (2H, m), 4.42 (2H, q), 4.15 (2H, m), 3.77 (2H, m), 3.76 (3H, s), 3.45 (3H, s), 1.38 (3H, t)

STEP 5: Hydrochloric acid (conc) (3 mL) was added dropwise to a stirred solution of E36 (505 mg, 1.446 mmol) in acetic acid (3 mL). The reaction mixture was warmed to 40° C. for 18 hours and was then allowed to cool to room temperature. The mixture was evaporated to dryness under reduced pressure (toluene was added to azeotrope out the final traces of water) to leave E37 as an off-white solid (443 mg).

1Hnmr(CDCl₃): 12.22 (1H, bs, OH), 7.16 (2H, m), 7.09 (2H, m), 4.18 (2H, t), 3.91 (3H, s), 3.78 (2H, t), 3.46 (3H, s)

STEP 6: A solution of E37 (400 mg, 1.245 mmol) and 5-amino-1-methyltetrazole (123.4 mg, 1.245 mmol) in dichloromethane (40 mL) were stirred together with DMAP (304.2 mg, 2.490 mmol) for 3 hours. A solution of 1-propanephosphonic acid cyclic anhydride (PPAA) in ethyl acetate (50 mass %; 1.584 g, 2.490 mmol) was then added, and the reaction mixture was stirred for 4 hours, then left to stand overnight.

The mixture was diluted with dichloromethane, washed with water and brine, then dried (MgSO₄) and evaporated under reduced pressure to afford the crude product as a white solid. This was purified by chromatography (CombiFlash RE 0-50% methanol in dichloromethane) to afford Compound 1.023 as a white solid (413 mg).

### EXAMPLE P12

### Experimental Procedure for the Synthesis of Compound 1.022

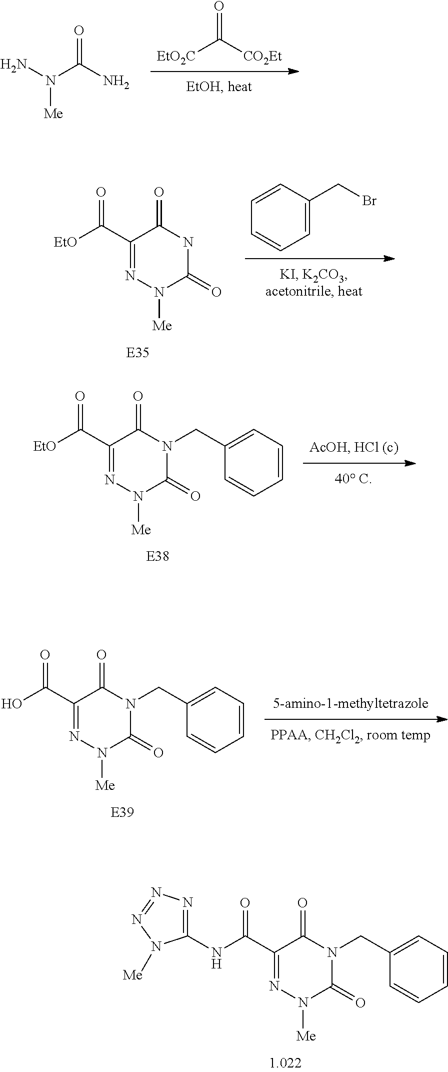

STEP 1: Diethyl 2-oxopropanedioate (4.7 mL, 29 mmol) was added in one portion to a stirred solution of 1-amino-1-methyl-urea (2.5 g, 28 mmol) in ethanol (40 mL). The reaction mixture was heated under reflux for a total of 25 hours (cooling to stand overnight on three occasions). The mixture was then evaporated under reduced pressure to leave a pale yellow liquid which crystallised on standing. This was purified by chromatography to afford E35 as a white solid (5.040 g).

1Hnmr(CDCl3): 4.42(q, 2H), 3.71(s, 3H), 1.39(t, 3H)

STEP 2: A stirred solution of E35 (310 mg, 1.557 mmol) in anhydrous acetonitrile (10 mL) was treated with benzyl bromide (0.19 mL, 1.6 mmol) and potassium iodide (299 mg, 1.80 mmol). After 10 mins, potassium carbonate (258 mg, 1.87 mmol) was added, and the reaction mixture was stirred at room temperature for 2 hours, then heated to 55° C. for 1 hour. The mixture was cooled to room temperature and ethyl acetate was added. The mixture was filtered, and the filtrate was evaporated under reduced pressure to leave a viscous yellow oil. This was purified by chromatography (CombiFlash RE 0-80% ethyl acetate in iso-hexane) to afford E38 as a colourless viscous oil (305mg)

1Hnmr(CDCl3): 7.52(d, 2H), 7.35-7.30(m, 3H), 5.12(s, 2H), 4.42(q, 2H), 3.72(s, 3H), 1.39(t, 3H)

STEP 3: A stirred solution of E38 (305 mg, 1.054 mmol) in acetic acid (4 mL) was treated dropwise with hydrochloric acid (conc) (1 mL). The mixture was then heated to 50° C. for 2 hours and was then cooled to room temperature and left to stand overnight. Analysis indicated that the reaction had not gone to completion so the mixture was heated to 50° C., with stirring, for a further 8 hours, and was then cooled and left to stand overnight. The mixture was evaporated to dryness under reduced pressure (toluene was added to azeotrope out the final traces of water) to leave E39 as a pale pink solid (244 mg).

1Hnmr(CDCl3): 12.30(broad, 1H), 7.54-7.49(m, 2H), 7.38-7.34(m, 3H), 5.19(s, 2H), 3.87(s, 3H)

STEP 4: A solution of E39 (244 mg, 0.934 mmol) and 5-amino-1-methyltetrazole (92 mg, 0.928 mmol) in dichloromethane (10 mL) were stirred together with DMAP (228 mg, 1.848 mmol) for 2 hours. A solution of 1-propanephosphonic acid cyclic anhydride (PPAA) in ethyl acetate (50 mass %; 1.21 mL, 1.90 mmol) was then added, and the reaction mixture was stirred for 1 hour, then left to stand overnight. Water was added and the mixture was stirred vigorously. The organic layer was separated, evaporated under reduced pressure and separated by chromatography (CombiFlash 0-5% methanol in dichloromethane) to afford Compound 1.022 as a white solid (266 mg).

### EXAMPLE P13

### Experimental Procedure for the Synthesis of Compound 1.004

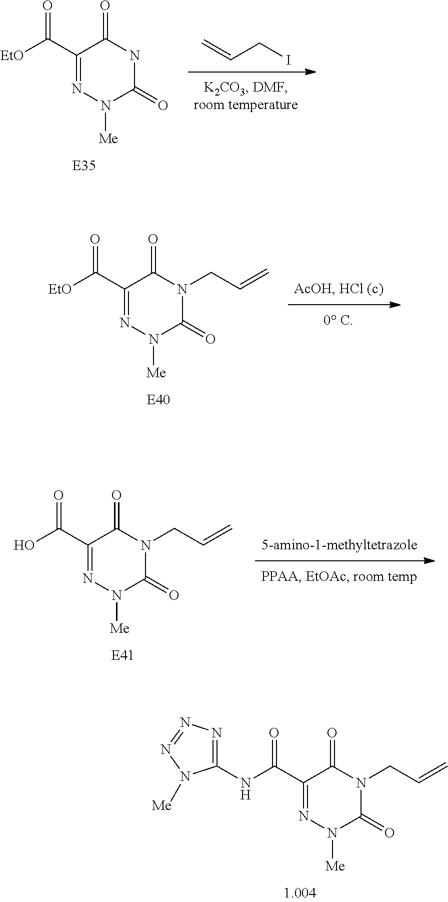

STEP 1: A solution of E35 (265 mg, 1.3305 mmol) in dimethylformamide (DMF) was treated with potassium carbonate (551 mg, 3.9867 mmol). The resulting slurry was stirred at room temperature and treated dropwise with allyl iodide (0.18 mL, 2.0 mmol). The reaction mixture was stirred at room temperature for 30 mins, and was then quenched with water. The resultant mixture was extracted with ethyl acetate, and the organic phase was dried (MgSO₄) and evaporated under reduced pressure to afford E40 as a colourless oil (211 mg).

1Hnmr(CDCl3): 5.91-5.80(m, 1H), 5.35(d, 1H), 5.28(d, 1H), 4.56(d, 2H), 4.42(q, 2H), 3.73(s, 3H), 1.40(t, 3H)

STEP 2: A stirred solution of E40 (211 mg, 0.882 mmol) in acetic acid (1 mL) was chilled in an ice/water bath and hydrochloric acid (conc) (1 mL) was added dropwise. The reaction mixture was stirred at this temp for 2 hours, and was then allowed to warm to room temperature and left to stand overnight. The mixture was evaporated under reduced pressure, and aq sodium bicarbonate was added. The mixture was extracted with ethyl acetate, and the aqueous phase was acidified with (conc) hydrochloric acid. The acidified aqueous phase was then extracted with ethyl acetate, and this extract was dried (MgSO₄) and evaporated under reduced pressure to afford E41 as a white solid (125 mg).

1Hnmr(CDCl3): 5.92-5.81(m, 1H), 5.41(d, 1H), 5.36(d, 1H), 4.62(d, 2H), 3.88(s, 3H)

STEP 3: A solution of E41 (125 mg, 0.592 mmol) and 5-amino-1-methyltetrazole (59 mg, 0.592 mmol) in ethyl acetate (10 mL) were stirred together with DMAP (144 mg, 1.167 mmol) for 1 hour. A solution of 1-propanephosphonic acid cyclic anhydride (PPAA) in ethyl acetate (50 mass %; 0.80 mL, 1.00 mmol) was then added, and the reaction mixture was stirred for 3 hours, then left to stand overnight. Water was added and the mixture was stirred vigorously. The organic layer was separated and evaporated under reduced pressure to leave a white solid. Trituration with iso-hexane afforded Compound 1.004 (73 mg).

### EXAMPLE P14

### Experimental Procedure for the Synthesis of Compound 1.005

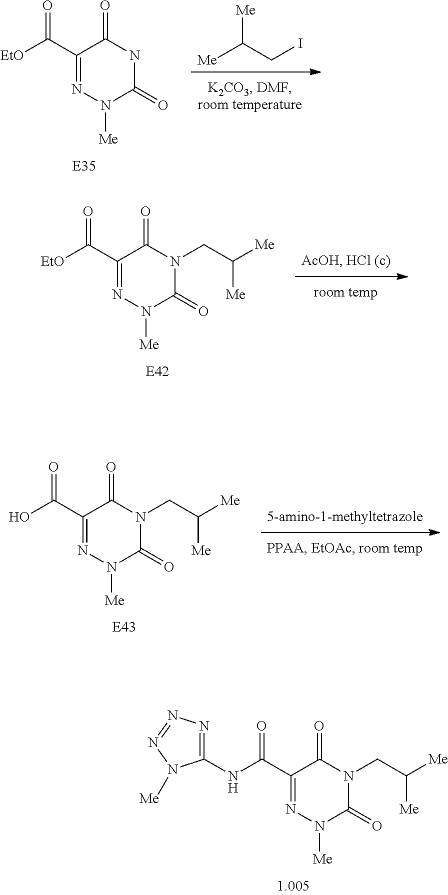 [SMILES: CCOC(=O)c1nn(C)c(=O)[nH]c1=O ; CC(C)CI ; CCOC(=O)c1nn(C)c(=O)n(CC(C)C)c1=O ; CC(C)Cn1c(=O)c(C(=O)O)nn(C)c1=O ; CC(C)Cn1c(=O)c(C(=O)Nc2nnnn2C)nn(C)c1=O]

STEP 1: A solution of E35 (300 mg, 1.506 mmol) in dimethylformamide (DMF; 5 mL) was treated with potassium carbonate (621 mg, 4.493 mmol). The resulting slurry was stirred at room temperature and treated dropwise with 1-iodo-2-methylpropane (0.26 mL, 2.3 mmol). The reaction mixture was stirred at room temperature for 30mins, and was then left to stand overnight. The mixture was quenched with water and extracted with dichloromethane, and the organic phase was evaporated under reduced pressure. The residue was purified by chromatography (CombiFlash

RE 0-30% ethyl acetate in hexane) to afford E42 as a colourless oil (140 mg).

1Hnmr(CDCl3): 4.42(q, 2H), 3.80(d, 2H), 3.72(s, 3H), 2.16(sept, 1H), 1.40(t, 3H), 0.94(d, 6H)

STEP 2: A stirred solution of E42 (140 mg, 0.548 mmol) in acetic acid (1 mL) was treated dropwise with hydrochloric acid (conc) (1 mL). The reaction mixture was stirred at this temp for 4 hours, and was then left to stand overnight. The mixture was evaporated under reduced pressure (toluene was added to azeotrope out the final traces of water) to leave E43 as a white solid (119 mg).

1Hnmr(CDCl3): 3.90-3.86 (m, 5H), 2.24-2.12 (m, 1H), 0.97(d, 6H)

STEP 3: A solution of E43 (119 mg, 0.524 mmol) and 5-amino-1-methyltetrazole (50 mg, 0.505 mmol) in ethyl acetate (5 mL) were stirred together with DMAP (128 mg, 1.037 mmol) for 1 hour. A solution of 1-propanephosphonic acid cyclic anhydride (PPAA) in ethyl acetate (50 mass %; 0.70 mL, 1.00 mmol) was then added, and the reaction mixture was stirred for 3 hours, then left to stand overnight. Water and ethyl acetate were added and the ethyl acetate layer was separated, and extracted with aqueous sodium bicarbonate solution. The sodium bicarbonate extract was acidified using dilute hydrochloric acid, and this was then extracted with ethyl acetate. The ethyl acetate extracts of the acidified solution were evaporated under reduced pressure (toluene was added to azeotrope out the final traces of water) to leave a white solid which was purified by chromatography (CombiFlash RE 0-5% methanol in dichloromethane) to afford Compound 1.005 as a white solid (31 mg).

### EXAMPLE P15

### Experimental Procedure for the Synthesis of Compound 1.010

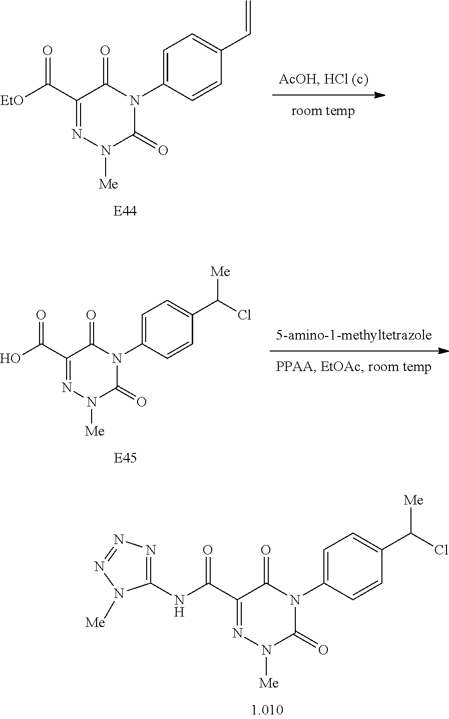 [SMILES: C=Cc1ccc(-n2c(=O)c(C(=O)OCC)nn(C)c2=O)cc1 ; CC(Cl)c1ccc(-n2c(=O)c(C(=O)O)nn(C)c2=O)cc1 ; CC(Cl)c1ccc(-n2c(=O)c(C(=O)Nc3nnnn3C)nn(C)c2=O)cc1]

STEP 1: A stirred solution of E44 (prepared from 4-vinylphenylboronic acid by a method analogous to that described in Example P11, Step 4; 213 mg, 0.707 mmol) in acetic acid (2 mL) was treated dropwise with hydrochloric acid (conc) (1 mL). The reaction mixture was stirred at this temp for 2 hours, and was then heated to 75° C. for 2 hours. After cooling, the mixture was evaporated under reduced pressure (toluene was added to azeotrope out the final traces of water) to leave E45 as a white solid (205 mg).

1Hnmr(DMSO): 7.62(d, 2H), 7.32(d, 2H), 5.42(q, 1H), 3.59(s, 3H), 1.84(d, 3H)

STEP 2: A solution of E45 (205 mg, 0.662 mmol) and 5-amino-1-methyltetrazole (65 mg, 0.660 mmol) in ethyl acetate (20 mL) were stirred together with DMAP (162 mg, 1.313 mmol) for 1 hour. A solution of 1-propanphosphonic acid cyclic anhydride (PPAA) in ethyl acetate (50 mass %; 0.83 mL, 1.30 mmol) was then added, and the reaction mixture was stirred for 1 hour, then left to stand overnight. Water and ethyl acetate were added and the ethyl acetate layer was separated, and extracted with aqueous sodium bicarbonate solution. The sodium bicarbonate extract was acidified using concentrated hydrochloric acid, and this was then extracted with ethyl acetate. The ethyl acetate extracts of the acidified solution were evaporated under reduced pressure (toluene was added to azeotrope out the final traces of water) to leave a white solid which was purified by chromatography (CombiFlash RE 0-5% methanol in dichloromethane) to afford Compound 1.010 as a white solid (45 mg).

### EXAMPLE P16

### Experimental Procedure for the Synthesis of Compound 1.008

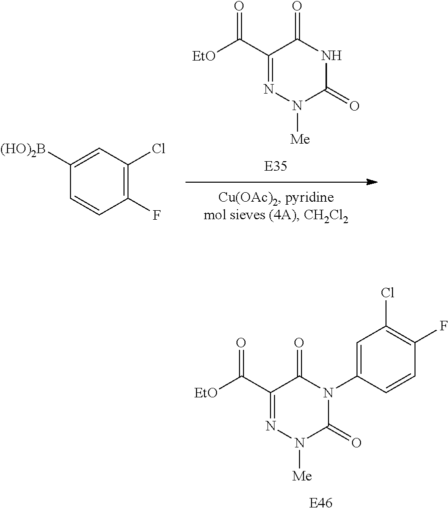 [SMILES: CCOC(=O)c1nn(C)c(=O)[nH]c1=O ; OB(O)c1ccc(F)c(Cl)c1 ; CCOC(=O)c1nn(C)c(=O)n(-c2ccc(F)c(Cl)c2)c1=O]

STEP 1: A stirred solution of 3-chloro-4-fluorophenylboronic acid (524 mg, 3.005 mmol) and E35 (300 mg, 1.506 mmol) and in dichloromethane (20 mL) was treated with powdered 4A molecular sieves (150 mg), pyridine (0.50 mL, 6.200 mmol) and copper (II) acetate (0.546 g, 3.006 mmol). The reaction mixture was stirred at room temperature for 22 hours. Ethyl acetate was then added and the mixture was filtered through Hi Flo (the copper salts were rinsed with more ethyl acetate). The green filtrate was evaporated under reduced pressure to leave a green solid residue. This was purified by chromatography (CombiFlash RE iso-hexane/ethyl acetate, 0% to 40%) to afford E46 as a white solid (293 mg).

1Hnmr(CDCl3): 7.35-7.29(m, 2H), 7.17-7.11(m, 1H), 4.43(q, 2H), 3.78(s, 3H), 1.40(t, 3H)

Compound E46 was then converted through to Compound 1.008 by methods analogous to those described in Steps 4 and 5 of Example P6.

### EXAMPLE P17

### Experimental Procedure for the Synthesis of Compound 1.013

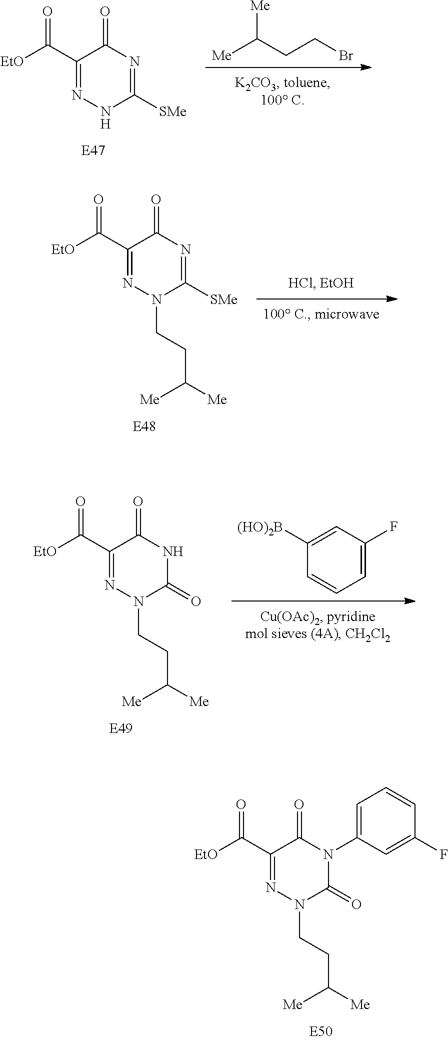 [SMILES: CCOC(=O)c1n[nH]c(SC)nc1=O ; CC(C)CCBr ; CCOC(=O)c1nn(CCC(C)C)c(SC)nc1=O ; CCOC(=O)c1nn(CCC(C)C)c(=O)[nH]c1=O ; OB(O)c1cccc(F)c1 ; CCOC(=O)c1nn(CCC(C)C)c(=O)n(-c2cccc(F)c2)c1=O]

STEP 1: A stirred suspension of E47 (300 mg, 1.394 mmol) in anhydrous toluene (10 mL) was treated with potassium carbonate (0.90 equiv., 1.255 mmol) under an argon atmosphere. The mixture was stirred at room temperature for 5.5 hours, then a solution of 1-bromo-3-methyl-butane (0.253 g, 1.673 mmol) in anhydrous toluene (1 mL) was added dropwise. The yellow mixture was heated at 100° C. for 26 hours, then cooled and left to stand overnight. Ethyl acetate was added, the mixture was filtered, and the insoluble material was washed with more ethyl acetate. The filtrate was then evaporated under reduced pressure to afford E48 as a yellow gum (114 mg).

1Hnmr(CDCl3): 4.42 (2H, q), 4.09 (2H, m), 2.59 (3H, s), 1.74 (2H, m), 1.69 (1H, m), 1.38 (3H, t), 0.99 (6H, d)

STEP 2: A microwave vial (2-5 mL capacity) containing a magnetic stirrer bar was charged with E48 (0.1 g, 0.4 mmol) and ethanol (2 mL) followed by hydrogen chloride (1.25 N in EtOH) (1.1 equiv., 0.4 mmol). The vial was sealed and irradiated in the microwave for 45 min at 100° C. The vessel pressure went up to 1 bar. The reaction mixture was evaporated under reduced pressure to afford E49 as a pale yellow gum (104 mg).

1Hnmr(CDCl3): 9.49 (1H, bs, NH), 4.42 (2H, q), 4.18 (1H, m), 4.07 (1H, m), 1.68 (3H, m), 1.40 (3H, t), 0.97 (6H, d)

STEP 3: A stirred solution of E49 (104 mg, 0.407 mmol) and 3-fluorophenyboronic acid (114 mg, 0.815 mmol) and in dichloromethane (15 mL) was treated with powdered 4A molecular sieves (50 mg), pyridine (129 mg, 61.63 mmol) and copper (II) acetate (148 mg, 0.815 mmol). The reaction mixture was stirred at room temperature for 22 hours. Analysis indicated that the reaction hadn't reached completion, so more 3-fluorophenyboronic acid (114 mg, 0.815 mmol) was added and the mixture was stirred for a further 22 hours. Ethyl acetate was then added and the mixture was filtered through Hi Flo (the copper salts were rinsed with more ethyl acetate). The green filtrate was evaporated under reduced pressure to leave a green solid residue. This was purified by chromatography (CombiFlash RE iso-hexane/ethyl acetate, 0% to 40%) to afford E50 as a white solid (70 mg).

1Hnmr(CDCl3): 7.49 (1H, m), 7.20 (1H, m), 6.98-7.06 (2H, m), 4.44 (2H, q), 4.12 (2H, m), 1.72 (2H, m), 1.70 (1H, m), 1.40 (3H, t), 0.99 (6H, d)

Compound E50 was then converted to Compound 1.008 by methods analogous to those described in Steps 4 and 5 of Example P6.

### EXAMPLE P18

### Experimental Procedure for the Synthesis of Compound 1.011

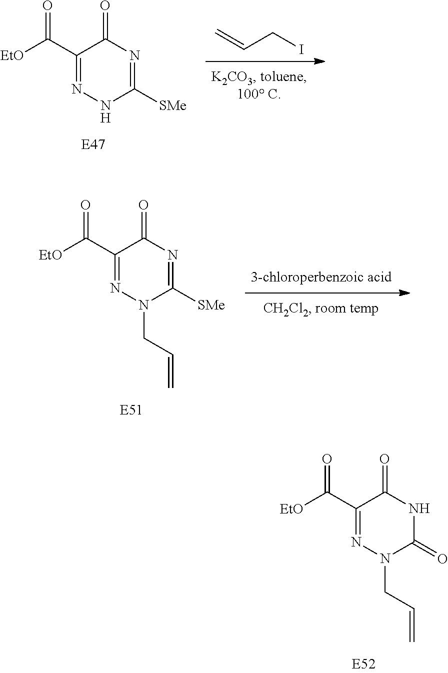

Step 1: A stirred suspension of E47 (200 mg, 0.929 mmol) in anhydrous toluene (4.5 mL) was treated with potassium carbonate (117 mg, 0.836 mmol) under an argon atmosphere. The mixture was stirred at room temperature for 2.75 hours, then a solution of allyl iodide (191 mg, 1.115 mmol) in anhydrous toluene (1 mL) was added dropwise. The yellow mixture was heated at 100° C. for 1 hour, then cooled and left to stand for 36 hours. Ethyl acetate was added, the mixture was filtered, and the insoluble material was washed with more ethyl acetate. The filtrate was then evaporated under reduced pressure to leave an orange gum which was purified by chromatography (CombiFlash RE iso-hexane/ethyl acetate, 0% to 100%) to afford E51 as a yellow oil (172 mg).

1Hnmr(CDCl3): 5.92 (1H, m), 5.32-5.41 (2H, m), 4.70 (2H, d), 4.41 (2H, q), 2.59 (3H, s), 1.38 (3H, t)

STEP 2: A stirred solution of E51 (170 mg, 0.666 mmol) in dichloromethane (20 mL) was treated portionwise with 3-chloroperbenzoic acid (361 mg, 1.465 mmol). The resulting solution was stirred at room temperature for 24 hours, after which it was diluted with more dichloromethane and washed with a 10% aqueous solution of Na₂S₂O₅, water, sodium bicarbonate solution, brine, and then dried (MgSO₄). Evaporation of the solvent under reduced pressure afforded a pale yellow oil which was purified by chromatography (CombiFlash RE iso-hexane/ethyl acetate, 0% to 100%) to afford E52 as a white solid (48 mg).

1Hnmr(CDCl3): 5.95 (1H, m), 5.35 (2H, m), 4.65 (2H, m), 4.43 (2H, q), 1.40 (3H, t) Compound E52 was then converted to Compound 1.011 by methods analogous to those described in Example P17, Step 3 and Example P6, steps 4 and 5.

### EXAMPLE P19

### Experimental Procedure for the Synthesis of Compound 1.123

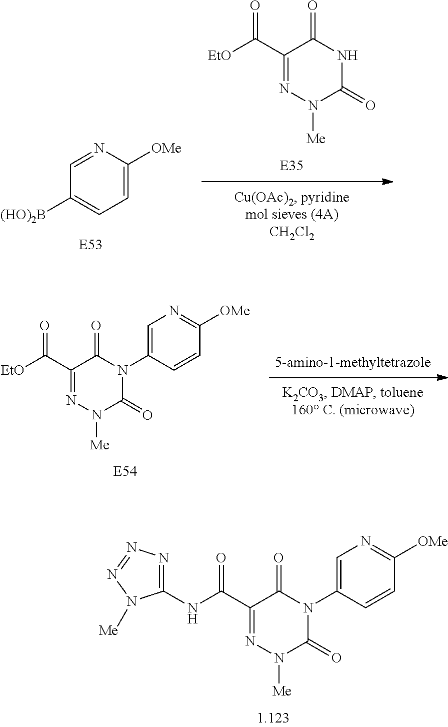 [SMILES: CCOC(=O)c1nn(C)c(=O)[nH]c1=O ; COc1ccc(B(O)O)cn1 ; CCOC(=O)c1nn(C)c(=O)n(-c2ccc(OC)nc2)c1=O ; COc1ccc(-n2c(=O)c(C(=O)Nc3nnnn3C)nn(C)c2=O)cn1]

STEP 1: A stirred suspension of E53 (765 mg, 5.00 mmol) and E35 (500 mg, 2.51 mmol) in dichloromethane (20 mL) was treated with powdered 4A molecular sieves (250 mg), pyridine (0.81 mL, 9.90 mmol) and copper II acetate (690 mg, 3.80 mmol). Tetrahydrofuran (THF; 10 mL) was added, and the mixture was stirred for 22 hours whilst air was bubbled through it. The mixture was filtered through Hi Flo and the insoluble residue was washed with ethyl acetate. The combined filtrates were diluted with ethyl acetate, washed with water, dried (MgSO4) and evaporated under reduced pressure to leave an orange gum. This was purified by chromatography (CombiFlash Rf, iso-hexane/ethyl acetate) to afford E54 as a cream solid (400 mg).

1Hnmr (CDCl3): 8.06(d, 1 H), 7.43(dd, 1 H), 6.87(d, 1 H), 4.43(q, 2 H), 3.98(s, 3 H), 3.78(s, 3 H), 1.40(t, 3 H)

STEP 2: A stirred mixture of E54 (30 mg, 0098 mmol), 5-amino-1-methyltetrazole (14 mg, 0.141 mmol), potassium carbonate (16 mg, 0.114 mmol) and N,N-dimethylaminopyridine (DMAP; 14 mg, 0.115 mmol) in toluene (4 mL) was heated to 160° C. in a microwave oven for 1 hour. The sample was cooled to room temperature and analysed by LCMS, which indicated that unreacted E54 was still present. Potassium carbonate (16 mg, 0.114 mmol) was added together with toluene (1 mL) and the reaction was heated again in the microwave for a further 45 minutes. The mixture was cooled, and the precipitate was filtered off and washed with iso-hexane.

The solid was then taken up in a mixture of dimethyl sulphoxide and methanol (9:1), filtered to remove insoluble material, and the filtrate was purified by mass-directed preparative HPLC, using a Waters Fraction Lynx system (eluting with water/trifluoroacetic acid and acetonitrile/acetic acid mixtures), to afford compound 1.123 as a white solid.

1Hnmr(CDCl3): 11.05(broad s, 1H), 8.12(d, 1H), 7.47(dd, 1H), 6.94(d, 1H), 4.08(s, 3H), 4.02(s, 3H), 3.96(s, 3H)

TABLE 1
Examples of herbicidal compounds of the present invention.

| Compound | | |
|---|---|---|
| Number | Structure | NMR |
| 1.001 | 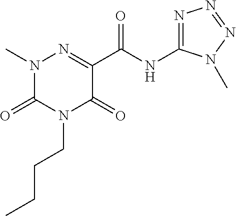 [SMILES: CCCCn1c(=O)c(C(=O)Nc2nnnn2C)nn(C)c1=O] | d6-DMSO: 3.79 (t, 2H,), 3.72 (s, 3H), 3.52 (s, 3H), 1.52 (m, 2H), 1.29 (m, 2H), 0.89 (t, 3H). |
| 1.002 | 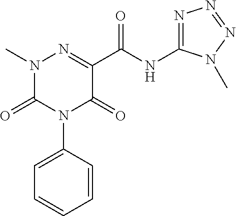 [SMILES: Cn1nnnc1NC(=O)c1nn(C)c(=O)n(-c2ccccc2)c1=O] | d6-DMSO: 11.35 (b s, 1H), 7.45-7.60 (m, 3H), 7.33 (m, 2H), 3.90 (s, 3H), 3.69 (s, 3H). |
| 1.003 | 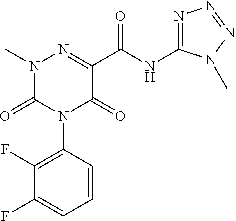 [SMILES: Cn1nnnc1NC(=O)c1nn(C)c(=O)n(-c2cccc(F)c2F)c1=O] | d6-DMSO: 11.37 (b s, 1H), 7.65 (m, 1H), 7.42 (m, 1H), 7.35 (m, 1H), 3.91 (s, 3H), 3.70 (s, 3H). |
| 1.004 | 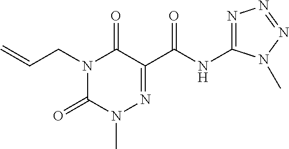 [SMILES: C=CCn1c(=O)c(C(=O)Nc2nnnn2C)nn(C)c1=O] | CDCl3: 11.25 (8, 1H). 5.93-5.82(m, 1H), 5.48- 5.35(m, 2H), 4.64(d, 2H), 4.07(s, 3H), 3.89(s, 3H) |
| 1.005 | 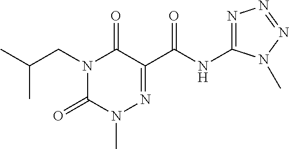 [SMILES: CC(C)Cn1c(=O)c(C(=O)Nc2nnnn2C)nn(C)c1=O] | CDCl3: 11.36(s, 1H), 4.07(s, 3H), 3.90(m, 2H), 3.88(s, 3H), 2.25-2.14(m, 1H), 0.98(d, 6H) |
| 1.006 | 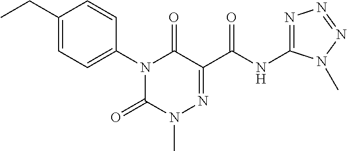 [SMILES: CCc1ccc(-n2c(=O)c(C(=O)Nc3nnnn3C)nn(C)c2=O)cc1] | CDCl3: 11.3(s, 1H), 7.41(d, 2H), 7.15(d, 2H), 4.07(s, 3H), 3.94(s, 3H), 2.76(q, 2H), 1.30(t,3H) |
| 1.007 | 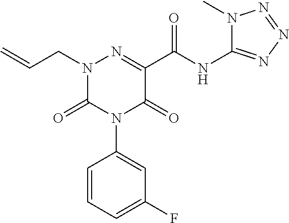 [SMILES: C=CCn1nc(C(=O)Nc2nnnn2C)c(=O)n(-c2cccc(F)c2)c1=O] | CDCl3: 11.02(b s1H), 7.57 (m, 1H), 7.27 (m, 11H), 7.04 (m, 2H), 6.04 (m, 1H), 5.45 (m, 2H), 4.85 (d, 2H), 4.07 (s, 3H) |
| 1.008 | 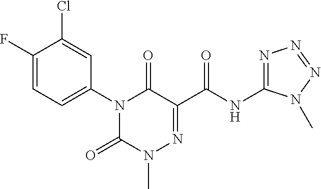 [SMILES: Cn1nnnc1NC(=O)c1nn(C)c(=O)n(-c2ccc(F)c(Cl)c2)c1=O] | (CDCl3 + DMSO): 11.17(s, 1H), 7.43(d, 1H), 7.36-7.32(m, 1H), 7.22(m, 1H), 4.05(s, 3H),3.89(s, 3H) |
| 1.009 | 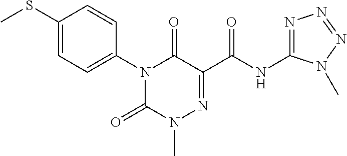 [SMILES: CSc1ccc(-n2c(=O)c(C(=O)Nc3nnnn3C)nn(C)c2=O)cc1] | CDCl3: 11.12(s, 1H), 7.40(d, 2H), 7.16(d, 2H), 4.06(s, 3H), 3.94(s, 3H), 2.54(s, 3H) |
| 1.010 | 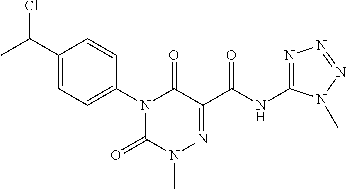 [SMILES: CC(Cl)c1ccc(-n2c(=O)c(C(=O)Nc3nnnn3C)nn(C)c2=O)cc1] | CDCl3: 11.09(b s, 1H), 7.65(d, 2H), 7.26(d, 2H), 5.15(q, 1H), 4.07(s, 3H), 3.95(s, 3H), 1.89(d, 3H) |
| 1.011 | 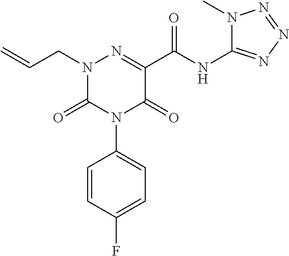 [SMILES: C=CCn1nc(C(=O)Nc2nnnn2C)c(=O)n(-c2ccc(F)cc2)c1=O] | CDCl3: 11.07(b s1H), 7.26-7.12 (m, 4H), 6.03 (m, 1H), 5.42 (m, 2H), 4.83 (d, 2H), 4.05 (s, 3H) |
| 1.012 | 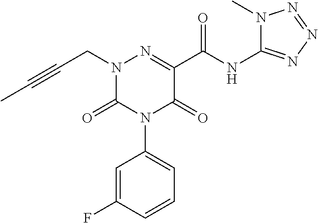 [SMILES: CC#CCn1nc(C(=O)Nc2nnnn2C)c(=O)n(-c2cccc(F)c2)c1=O] | CDCl3: 11.04 (b s, 1H), 7.56 (m, 1H), 7.26 (m, 1H), 7.13-7.00 (m, 2H), 4.95 (m, 2H), 4.06 (s, 3H), 1.85 (t, 3H) |
| 1.013 | 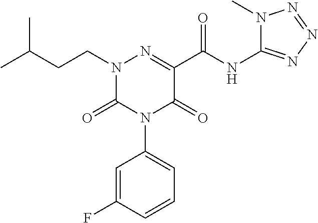 [SMILES: CC(C)CCn1nc(C(=O)Nc2nnnn2C)c(=O)n(-c2cccc(F)c2)c1=O] | CDCl3: 11.06 (b s, 1H), 7.56 (m, 1H), 7.26 (m, 1H), 7.09 (d, 1H), 7.04 (d, 1H), 4.27 (m, 2H), 4.06 (s, 3H), 1.78 (m, 2H), 1.71 (m, 1H), 0.99 (d, 6H) |
| 1.014 | 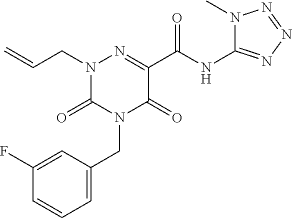 [SMILES: C=CCn1nc(C(=O)Nc2nnnn2C)c(=O)n(Cc2cccc(F)c2)c1=O] | CDCl3: 11.24 (b s, 1H), 7.35-7.20 (m, 3H), 7.03 (m, 1H), 5.98 (m, 1H), 5.37 (m, 2H), 5.16 (s, 2H), 4.80 (d, 2H), 4.04 (s, 3H) |
| 1.015 | 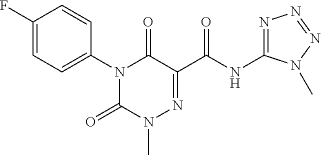 [SMILES: Cn1nnnc1NC(=O)c1nn(C)c(=O)n(-c2ccc(F)cc2)c1=O] | CDCl3: 11.31(s, 1H), 7.40-7.38(m, 4H), 3.92(s, 3H), 3.69(s, 3H) |
| 1.016 | 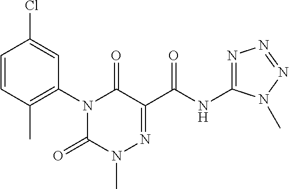 [SMILES: Cc1ccc(Cl)cc1-n1c(=O)c(C(=O)Nc2nnnn2C)nn(C)c1=O] | CDCl3: 11.06(s, 1H), 7.43(dd, 1H), 7.36(d, 1H), 7.17(d, 1H), 4.07(s, 3H), 3.94(s, 3H), 2.14(s, 3H) |
| 1.017 | 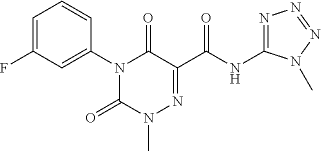 [SMILES: Cn1nnnc1NC(=O)c1nn(C)c(=O)n(-c2cccc(F)c2)c1=O] | d6-DMSO: 11.30(s, 1H), 7.60(q, 1H), 7.37(dt, 1H), 7.29-7.19(m, 2H), 3.90(s, 3H), 3.67(s, 3H) |
| 1.018 | 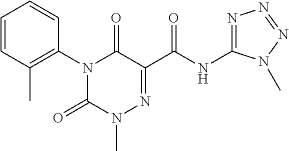 [SMILES: Cc1ccccc1-n1c(=O)c(C(=O)Nc2nnnn2C)nn(C)c1=O] | CDCl3: 11.14(s, 1H), 7.50-7.37(m, 3H), 7.13(d, 1H), 4.08(s, 3H), 3.96(s, 3H), 2.18(s, 3H) |
| 1.019 | 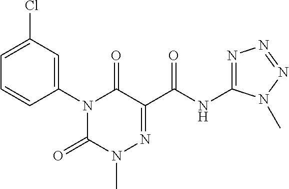 [SMILES: Cn1nnnc1NC(=O)c1nn(C)c(=O)n(-c2cccc(Cl)c2)c1=O] | CDCl3: 10.97(s, 1H), 7.55-7.53(m, 2H), 7.27(d, 1H), 7.19-7.15(m, 1H), 4.07(s, 3H), 3.94(s, 3H) |
| 1.020 | 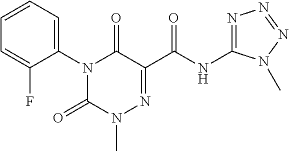 [SMILES: Cn1nnnc1NC(=O)c1nn(C)c(=O)n(-c2ccccc2F)c1=O] | CDCl3: 11.08(s, 1H), 7.59-7.51(m, 1H), 7.36- 7.28(m, 3H), 4.04(s, 3H), 3.92(s, 3H) |
| 1.021 | 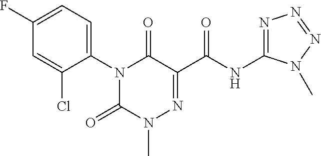 [SMILES: Cn1nnnc1NC(=O)c1nn(C)c(=O)n(-c2ccc(F)cc2Cl)c1=O] | CDCl3: 10.99(s, 1H), 7.41-7.38(m, 1H), 7.32- 7.29(m, 1H), 7.22-7.18(m, 1H), 4.08(s, 3H), 3.96(s, 3H) |
| 1.022 | 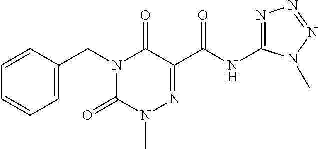 [SMILES: Cn1nnnc1NC(=O)c1nn(C)c(=O)n(Cc2ccccc2)c1=O] | CDCl3: 11.30(s, 1H), 7.54-7.51(m, 2H), 7.40- 7.33(m, 3H), 5.21(s, 2H), 4.06(s, 3H), 3.89(s, 3H) |
| 1.023 | 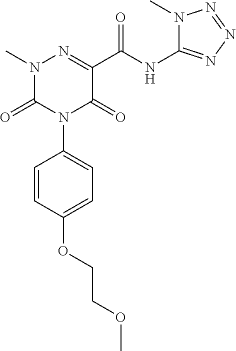 [SMILES: COCCOc1ccc(-n2c(=O)c(C(=O)Nc3nnnn3C)nn(C)c2=O)cc1] | CDCl3: 11.16 (b s, 1H), 7.17 (m, 2H), 7.10 (m, 2H), 4.19 (t, 2H), 4.07 (s, 3H), 3.94 (s, 3H), 3.79 (t, 2H), 3.47 (s, 3H) |
| 1.024 | 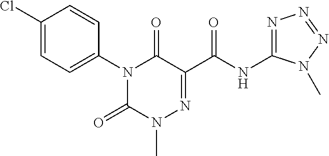 [SMILES: Cn1nnnc1NC(=O)c1nn(C)c(=O)n(-c2ccc(Cl)cc2)c1=O] | d3-MeCN: 10.68(b s, 1H), 7.63(d, 2H), 7.32(d, 2H), 3.95(s, 3H), 3.29(s, 3H) |
| 1.025 | 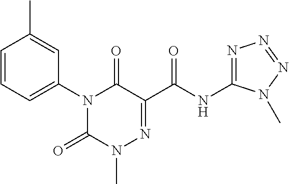 [SMILES: Cc1cccc(-n2c(=O)c(C(=O)Nc3nnnn3C)nn(C)c2=O)c1] | d3-MeCN: 10.78(b s, 1H), 7.47(t, 1H), 7.38(d, 1H), 7.13-7.09(m, 2H), 3.94(s, 3H), 3.79(s, 3H), 2.44(s, 3H) |
| 1.026 | 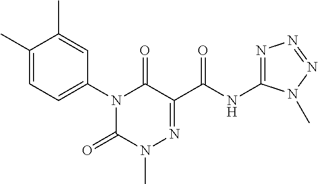 [SMILES: Cc1ccc(-n2c(=O)c(C(=O)Nc3nnnn3C)nn(C)c2=O)cc1C] | d3-MeCN: 10.78(b s, 1H), 7.36(d, 1H), 7.08-7.01(m, 2H), 3.96(s, 1H), 3.78(s, 3H), 2.35(s, 3H), 2.33(s, 3H) |
| 1.027 | 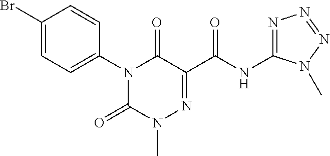 [SMILES: Cn1nnnc1NC(=O)c1nn(C)c(=O)n(-c2ccc(Br)cc2)c1=O] | d3-MeCN: 10.65(b s, 1H), 7.78(d, 2H), 7.24(d, 2H), 3.95(s, 3H), 3.79(s, 3H) |
| 1.028 | 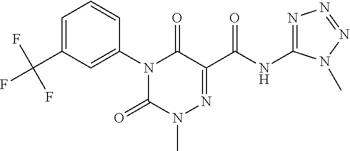 [SMILES: Cn1nnnc1NC(=O)c1nn(C)c(=O)n(-c2cccc(C(F)(F)F)c2)c1=O] | d3-MeCN: 10.55(b s, 1H), 7.89(d, 1H), 7.83(t, 1H), 7.16-7.19(m, 2H), 3.95(s, 3H), 3.81(s, 3H) |
| 1.029 | 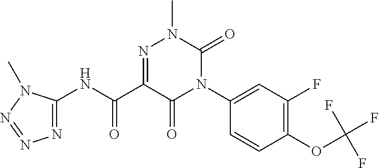 [SMILES: Cn1nnnc1NC(=O)c1nn(C)c(=O)n(-c2ccc(OC(F)(F)F)c(F)c2)c1=O] | d3-MeCN: 10.53(b s, 1H), 7.67(t, 1H), 7.46(dd, 1H), 7.27(m, 1H), 3.94(s, 3H), 3.79(s, 3H) |
| 1.030 | 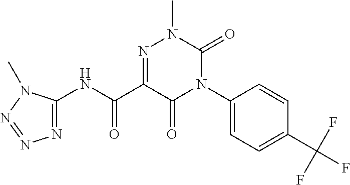 [SMILES: Cn1nnnc1NC(=O)c1nn(C)c(=O)n(-c2ccc(C(F)(F)F)cc2)c1=O] | d3-MeCN: 10.58(bs, 1H), 7.94(d, 2H), 7.55(d, 2H), 3.96(s, 3H), 3.82(s, 3H) |
| 1.031 | 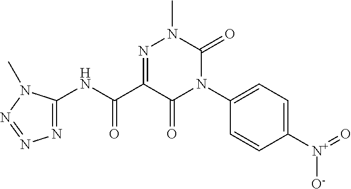 [SMILES: Cn1nnnc1NC(=O)c1nn(C)c(=O)n(-c2ccc([N+](=O)[O-])cc2)c1=O] | d3-MeCN: 10.50 (b, 1H), 8.44(d, 2H), 7.59(d, 2H), 3.94(s, 3H), 3.79(s, 3H) |
| 1.032 | 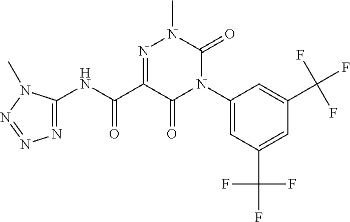 [SMILES: Cn1nnnc1NC(=O)c1nn(C)c(=O)n(-c2cc(C(F)(F)F)cc(C(F)(F)F)c2)c1=O] | d3-MeCN: 10.45(b, 1H), 8.75(s, 1H), 7.96(s, 2H), 3.97(s, 3H), 3.81(s, 3H) |
| 1.033 | 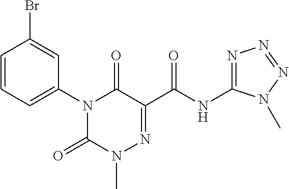 [SMILES: Cn1nnnc1NC(=O)c1nn(C)c(=O)n(-c2cccc(Br)c2)c1=O] | d3-MeCN: 10.58(b s, 1H), 7.74(dd, 1H), 7.57-7.50(m, 2H), 7.34(dd, 1H), 3.96(s, 3H), 3.81(s, 3H) |
| 1.034 | 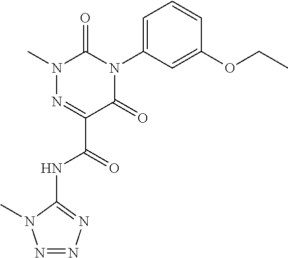 [SMILES: CCOc1cccc(-n2c(=O)c(C(=O)Nc3nnnn3C)nn(C)c2=O)c1] | d3-MeCN: 10.71(b s, 1H), 7.49(t, 1H), 7.08(dd, 1H), 6.87(dd, 1H), 6.84(m, 1H), 4.08(q, 2H), 3.96(s, 3H), 3.78(s, 3H), 1.39(t,3H) |
| 1.035 | 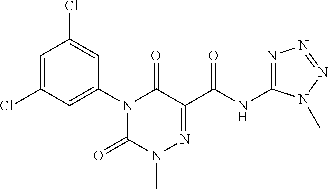 [SMILES: Cn1nnnc1NC(=O)c1nn(C)c(=O)n(-c2cc(Cl)cc(Cl)c2)c1=O] | d3-MeCN: 10.55(b s, 1H), 7.69(s, 1H), 7.36(s, 2H), 3.95(s, 3H), 3.79(s, 3H) |
| 1.036 | 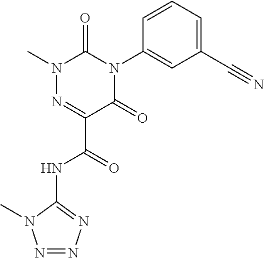 [SMILES: Cn1nnnc1NC(=O)c1nn(C)c(=O)n(-c2cccc(C#N)c2)c1=O] | d3-MeCN: 10.55(b s, 1H), 7.95(dd, 1H), 7.78(t, 1H), 7.70(m, 1H), 7.65(m, 1H), 3.96(s, 3H), 3.79(s, 3H) |
| 1.037 | 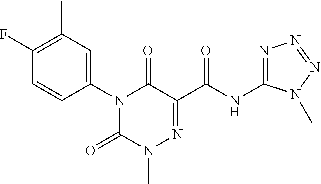 [SMILES: Cc1cc(-n2c(=O)c(C(=O)Nc3nnnn3C)nn(C)c2=O)ccc1F] | d3-MeCN: 10.70(b s, 1H), 7.26(t, 1H), 7.22-7.11(m, 2H), 3.94(s, 3H), 3.78(s, 3H), 2.36(s, 3H) |
| 1.038 | 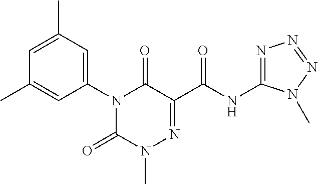 [SMILES: Cc1cc(C)cc(-n2c(=O)c(C(=O)Nc3nnnn3C)nn(C)c2=O)c1] | d3-MeCN: 10.78(b s, 1H), 10.22(s, 1H), 6.92(s, 2H), 3.96(s, 3H), 3.78(s, 3H), 2.37(s, 6H) |
| 1.039 | 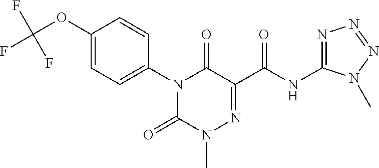 [SMILES: Cn1nnnc1NC(=O)c1nn(C)c(=O)n(-c2ccc(OC(F)(F)F)cc2)c1=O] | d3-MeCN: 10.65(b, 1H), 7.53(t, 2H), 7.45(d, 2H), 3.97(s, 3H), 3.79(s, 3H), |
| 1.040 | 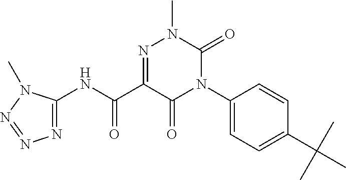 [SMILES: Cn1nnnc1NC(=O)c1nn(C)c(=O)n(-c2ccc(C(C)(C)C)cc2)c1=O] | d3-MeCN: 10.80(b, 1H), 7.65(d, 2H), 7.26(d, 2H), 3.95(s, 3H), 3.79(s, 3H), 1.39(s, 9H) |
| 1.041 | 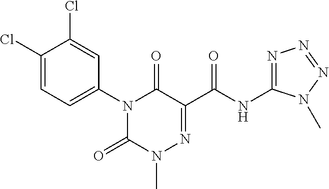 [SMILES: Cn1nnnc1NC(=O)c1nn(C)c(=O)n(-c2ccc(Cl)c(Cl)c2)c1=O] | d3-MeCN: 10.55(b, 1H), 7.77(d, 1H), 7.53(d, 1H), 7.29(dd, 1H), 3.96(s, 3H), 3.79(s, 3H) |
| 1.042 | 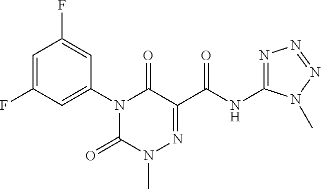 [SMILES: Cn1nnnc1NC(=O)c1nn(C)c(=O)n(-c2cc(F)cc(F)c2)c1=O] | d3-MeCN: 10.55(b, 1H), 7.19(m, 1H), 7.02(m, 2H), 3.96(s, 3H), 3.79(s, 3H) |
| 1.043 | 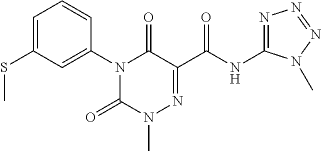 [SMILES: CSc1cccc(-n2c(=O)c(C(=O)Nc3nnnn3C)nn(C)c2=O)c1] | d3-MeCN: 10.65(b s, 1H), 7.5l(t, 1H), 7.45(dd, 1H), 7.19(m, 1H), 7.09(dd, 1H), 3.96(s, 3H), 3.79(s, 3H), 2.53(s, 3H) |
| 1.044 | 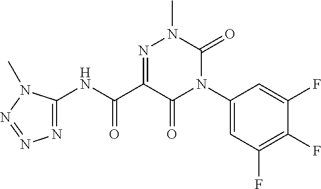 [SMILES: Cn1nnnc1NC(=O)c1nn(C)c(=O)n(-c2cc(F)c(F)c(F)c2)c1=O] | d3-MeCN: 7.18(m, 2H), 3.95(s, 3H), 3.77(s, 3H), |
| 1.045 | 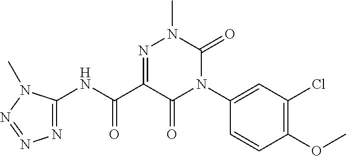 [SMILES: COc1ccc(-n2c(=O)c(C(=O)Nc3nnnn3C)nn(C)c2=O)cc1Cl] | d3-MeCN: 10.65(b, 1H), 7.34(m, 1H), 7.26(m, 2H), 3.97(s, 3H), 3.93(s, 3H), 3.79(s, 3H) |
| 1.046 | 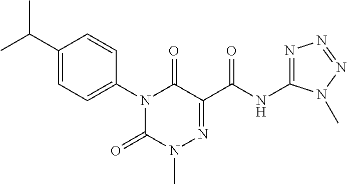 [SMILES: CC(C)c1ccc(-n2c(=O)c(C(=O)Nc3nnnn3C)nn(C)c2=O)cc1] | d3-MeCN: 10.75(b s, 1H) 7.47(d, 2H), 7.24(d, 2H), 3.95(s, 3H), 3.78(s, 3H), 3.05(m, 1H), 1.31(d, 6H) |
| 1.047 | 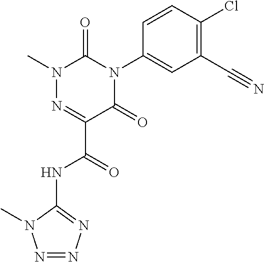 [SMILES: Cn1nnnc1NC(=O)c1nn(C)c(=O)n(-c2ccc(Cl)c(C#N)c2)c1=O] | d3-MeCN: 7.85(d, 1H), 7.77(d, 1H), 7.62(dd, 1H), 3.97(s, 3H), 3.79(s, 3H) |
| 1.048 | 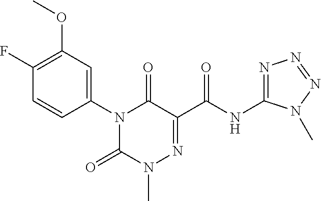 [SMILES: COc1cc(-n2c(=O)c(C(=O)Nc3nnnn3C)nn(C)c2=O)ccc1F] | d3-MeCN: 10.67(b, 1H), 7.31(dd, 1H), 7.07(dd, 1H), 6.89(m, 1H), 3.97(s, 3h), 3.88(s, 3H), 3.79(s, 3H) |
| 1.049 | 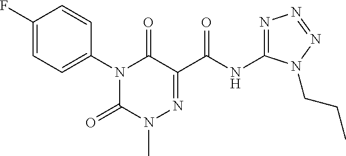 [SMILES: CCCn1nnnc1NC(=O)c1nn(C)c(=O)n(-c2ccc(F)cc2)c1=O] | CDCl3: 11.01(s, 1H), 7.31-7.28(m, 4H), 4.33(t, 2H), 3.95(s, 3H), 2.06- 1.95 (m, 2H), 0.98(t, 3H) |
| 1.050 | 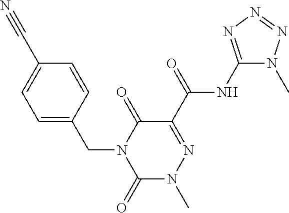 [SMILES: Cn1nnnc1NC(=O)c1nn(C)c(=O)n(Cc2ccc(C#N)cc2)c1=O] | d3-MeCN: 10.72(b s, 1H), 7.74(d, 2H), 7.59(d, 2H), 5.19(s, 2H), 3.95(s, 3H), 3.76(s, 3H) |
| 1.051 | 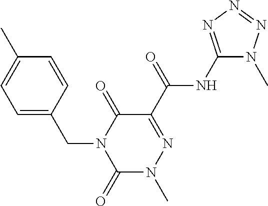 [SMILES: Cc1ccc(Cn2c(=O)c(C(=O)Nc3nnnn3C)nn(C)c2=O)cc1] | d3-MeCN: 7.36(d, 2H), 7.19(d, 2H), 5.10(s, 2H), 3.94(s, 3H), 3.75(s, 3H), 2.31(s, 3H) |
| 1.052 | 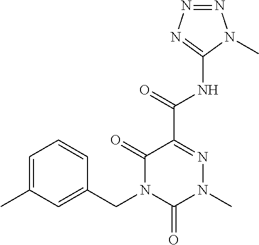 | d3-MeCN: 10.83(b s, 1H), 7.27-7.21(m, 3H), 7.14(m, 1H), 5.11(s, 2H), 3.95(s, 3H), 3.77(s, 3H), 2.34(s, 3H) |
| 1.053 | 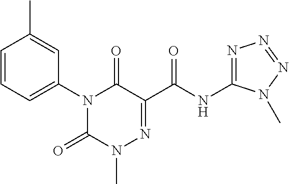 [SMILES: Cc1cccc(-n2c(=O)c(C(=O)Nc3nnnn3C)nn(C)c2=O)c1] | d3-MeCN: 10.81(b s, 1H), 7.36-7.11(m, 4H), 5.13(s, 2H), 3.96(s, 3H), 3.78(s, 3H), 2.42(s, 3H) |
| 1.054 | 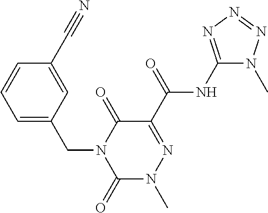 [SMILES: Cn1nnnc1NC(=O)c1nn(C)c(=O)n(Cc2cccc(C#N)c2)c1=O] | d3-MeCN: 10.72(b s, 1H), 7.80-7.72(m, 2H), 7.70(dd, 1H), 7.53(t, 1H), 5.19(s, 2H), 3.96(s, 3H), 3.76(s, 3H) |
| 1.055 | 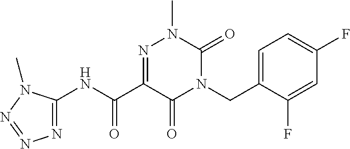 [SMILES: Cn1nnnc1NC(=O)c1nn(C)c(=O)n(Cc2ccc(F)cc2F)c1=O] | d3-MeCN: 10.73(b s, 1H), 7.45(m, 1H), 6.98(m, 2H), 5.18(s, 2H), 3.94(s, 3H), 3.74(s, 3H) |
| 1.056 | 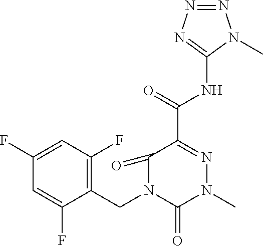 [SMILES: Cn1nnnc1NC(=O)c1nn(C)c(=O)n(Cc2c(F)cc(F)cc2F)c1=O] | d3-MeCN: 10.68(b s, 1H), 6.87(m, 2H), 5.19(s, 2H), 3.96(s, 3H), 3.73(s, 3H) |
| 1.057 | 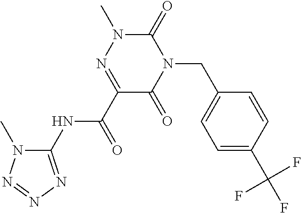 [SMILES: Cn1nnnc1NC(=O)c1nn(C)c(=O)n(Cc2ccc(C(F)(F)F)cc2)c1=O] | d3-MeCN: 10.75(b, 1H), 7.69(d, 2H), 7.64(d, 2H), 5.20(s, 2H), 3.95(s, 3H), 3.76(s, 3H) |
| 1.058 | 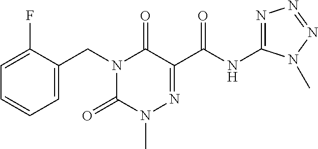 [SMILES: Cn1nnnc1NC(=O)c1nn(C)c(=O)n(Cc2ccccc2F)c1=O] | d3-MeCN: 10.75(b s, 1H), 7.44-7.34(m, 2H), 7.20- 7.11(m, 2H), 5.21(s, 2H), 3.95(s, 3H), 3.77(s, 3H) |
| 1.059 | 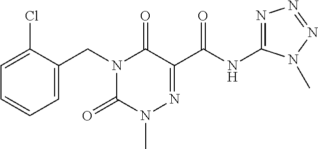 [SMILES: Cn1nnnc1NC(=O)c1nn(C)c(=O)n(Cc2ccccc2Cl)c1=O] | d3-MeCN: 10.69(b s, 1H), 7.49(dd, 1H), 7.37-7.23(m, 3H), 5.25(s, 2H), 3.94(s, 3H), 3.79(s, 3H) |
| 1.060 | 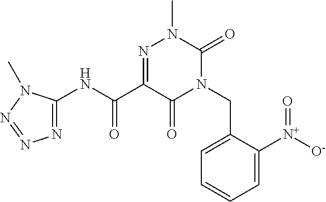 [SMILES: Cn1nnnc1NC(=O)c1nn(C)c(=O)n(Cc2ccccc2[N+](=O)[O-])c1=O] | d3-MeCN: 10.68(b s, 1H), 8.11(dd, 1H), 7.67(dt, 1H), 7.57(dt, 1H), 7.47(dd, 1H), 5.50(s, 2H), 3.96(s, 3H), 3.76(s, 3H) |
| 1.061 | 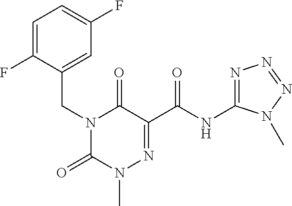 [SMILES: Cn1nnnc1NC(=O)c1nn(C)c(=O)n(Cc2cc(F)ccc2F)c1=O] | d3-MeCN: 10.70(b s, 1H), 7.71-7.54(m, 3H)5.19(s, 2H), 3.95(s, 3H), 3.74(s, 3H) |
| 1.062 | 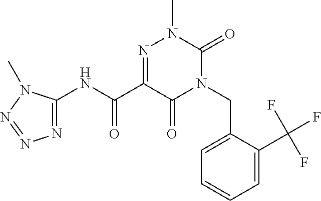 [SMILES: Cn1nnnc1NC(=O)c1nn(C)c(=O)n(Cc2ccccc2C(F)(F)F)c1=O] | d3-MeCN: 10.68(b s, 1H), 7.80(d, 1H), 7.58(t, 1H), 7.52(t, 1H), 7.29(d, 1H), 5.33(s, 2H), 3.96(s, 3H), 3.80(s, 3H) |
| 1.063 | 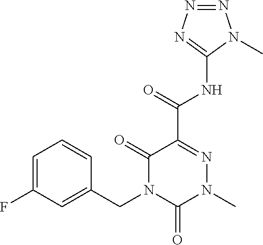 [SMILES: Cn1nnnc1NC(=O)c1nn(C)c(=O)n(Cc2cccc(F)c2)c1=O] | d3-MeCN: 10.75(b s, 1H), 7.39(m, 1H), 7.29(m, 1H), 7.20(m, 1H), 7.09(m, 1H), 5.14(s, 2H), 3.94(s, 3H), 3.75(s, 3H) |
| 1.064 | 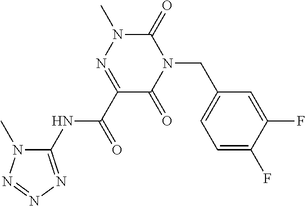 [SMILES: Cn1nnnc1NC(=O)c1nn(C)c(=O)n(Cc2ccc(F)c(F)c2)c1=O] | d3-MeCN: 10.70(b s, 1H), 7.38(m, 1H), 7.30-7.23(m, 2H), 5.12(s, 2H), 3.97(s, 3H), 3.75(s, 3H) |
| 1.065 | 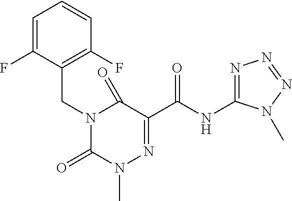 [SMILES: Cn1nnnc1NC(=O)c1nn(C)c(=O)n(Cc2c(F)cccc2F)c1=O] | d3-MeCN: 10.70(b s, 1H), 7.39(m, 1H), 7.01(m, 2H), 5.25(s, 2H), 3.94(s, 3H), 3.74(s, 3H) |
| 1.066 | 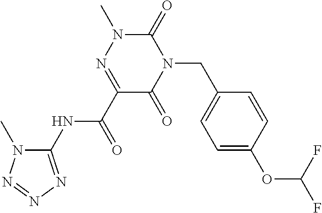 [SMILES: Cn1nnnc1NC(=O)c1nn(C)c(=O)n(Cc2ccc(OC(F)F)cc2)c1=O] | d3-MeCN: 10.78(b s, 1H), 7.52(d, 2H), 7.15(d, 2H), 6.78(t, 1H), 3.95(s, 3H), 3.74(s, 3H) |
| 1.067 | 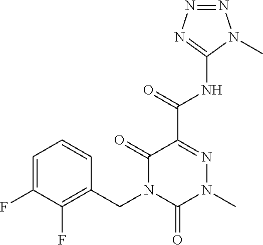 [SMILES: Cn1nnnc1NC(=O)c1nn(C)c(=O)n(Cc2cccc(F)c2F)c1=O] | d3-MeCN: 10.69(b s, 1H), 7.29-7.09(m, 3H), 5.22(s, 2H), 3.95(s, 3H), 3.74(s, 3H) |
| 1.068 | 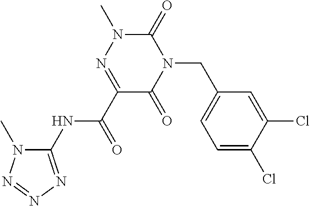 [SMILES: Cn1nnnc1NC(=O)c1nn(C)c(=O)n(Cc2ccc(Cl)c(Cl)c2)c1=O] | d3-MeCN: 10.72(b, 1H), 7.61(d, 1H), 7.53(d, 1H), 7.41(dd, 1H), 7.51(s, 2H), 3.95(s, 3H), 3.75(s, 3H) |
| 1.069 | 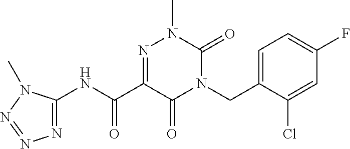 [SMILES: Cn1nnnc1NC(=O)c1nn(C)c(=O)n(Cc2ccc(F)cc2Cl)c1=O] | d3-MeCN: 10.65(b, 1H), 7.33-7.27(m, 2H), 7.09(m, 1H), 5.21(s, 2H), 3.95(s, 2H), 3.76(s, 3H) |
| 1.070 | 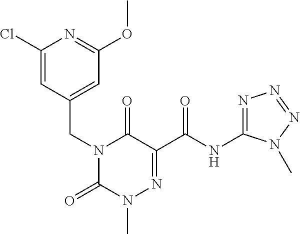 [SMILES: COc1cc(Cn2c(=O)c(C(=O)Nc3nnnn3C)nn(C)c2=O)cc(Cl)n1] | d3-MeCN: 10.68(b, 1H), 7.02(s, 1H), 6.75(s, 1H), 5.08(s, 2H), 3.96(s, 3H), 3.87(s, 3H), 3.75(s, 3H) |
| 1.071 | 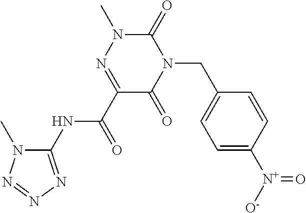 [SMILES: Cn1nnnc1NC(=O)c1nn(C)c(=O)n(Cc2ccc([N+](=O)[O-])cc2)c1=O] | d3-MeCN: 10.69(b, 1H), 8.22(d, 2H), 7.66(d, 2H), 5.22(s, 2H), 3.95(s, 3h), 3.75(s, 3H) |
| 1.072 | 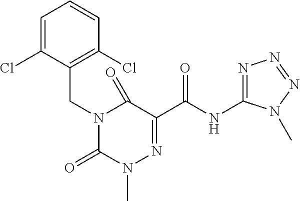 [SMILES: Cn1nnnc1NC(=O)c1nn(C)c(=O)n(Cc2c(Cl)cccc2Cl)c1=O] | d3-MeCN: 10.68(b s, 1H), 7.43(d, 2H), 7.29(t, 1H), 5.41(s, 2H), 3.94(s, 3H), 3.71(s, 3H) |
| 1.073 | 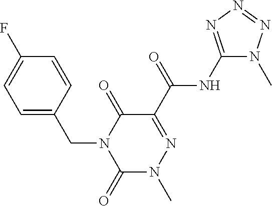 [SMILES: Cn1nnnc1NC(=O)c1nn(C)c(=O)n(Cc2ccc(F)cc2)c1=O] | d3-MeCN: 7.48(m, 2H), 7.09(m, 2H), 5.12(s, 2H), 3.94(s, 3H), 3.75(s, 3H) |
| 1.074 | 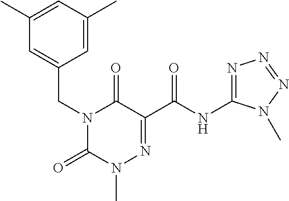 [SMILES: Cc1cc(C)cc(Cn2c(=O)c(C(=O)Nc3nnnn3C)nn(C)c2=O)c1] | d3-MeCN: 10.80(b s, 1H), 7.03(s, 2H), 6.96(s, 1H), 5.08(s, 2H), 3.96(s, 3H), 3.76(s, 3H), 2.29(s, 6H) |
| 1.075 | 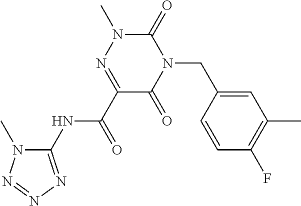 | d3-MeCN: 10.78(b s, 1H), 7.36-7.27(m, 2H), 7.02(, t, 1H), 5.08(s, 2H),3.94(s, 3H), 3.75(s, 3H), 2.22(s, 3H) |
| 1.076 | 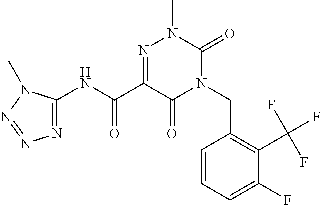 [SMILES: Cn1nnnc1NC(=O)c1nn(C)c(=O)n(Cc2cccc(F)c2C(F)(F)F)c1=O] | d3-MeCN: 10.64(b s, 1H), 7.58(m, 1H), 7.38(m, 1H), 7.09(m, 1H), 5.33(s, 2H), 3.95(s, 3H), 3.77(s, 3H) |
| 1.077 | 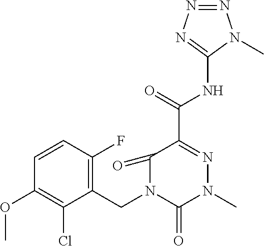 [SMILES: COc1ccc(F)c(Cn2c(=O)c(C(=O)Nc3nnnn3C)nn(C)c2=O)c1Cl] | CDCl3: 11.18(s, 1H), 6.98(t, 1H), 6.88(m, 1H), 5.40(s, 2H), 4.06(s, 3H), 3.88(s, 3H), 3.84(s, 3H) |
| 1.078 | 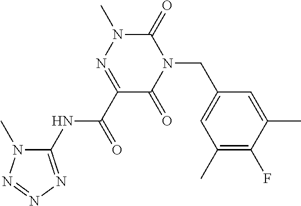 | d3-MeCN: 10.80(b, 1H), 7.12(m, 2H), 5.05(s, 2H), 3.95(s, 3H), 3.74(s, 3H), 2.20(s, 6H) |
| 1.079 | 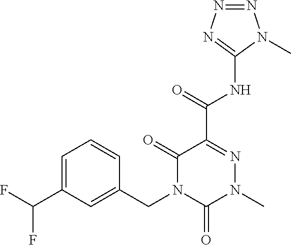 [SMILES: Cn1nnnc1NC(=O)c1nn(C)c(=O)n(Cc2cccc(C(F)F)c2)c1=O] | d3-MeCN: 10.78(b s, 1H), 7.66-7.58(m, 2H), 7.55- 7.47(m, 2H), 6.80(t, 1H), 5.18(s, 2H), 3.95(s, 3H), 3.74(s, 3H) |
| 1.080 | 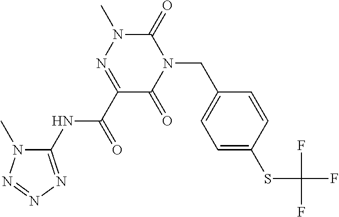 [SMILES: Cn1nnnc1NC(=O)c1nn(C)c(=O)n(Cc2ccc(SC(F)(F)F)cc2)c1=O] | d3-MeCN: 10.75(b, 1H), 7.70(d, 2H), 7.57(d, 2H), 5.19(s, 2H), 3.94(s, 3H), 3.75(s, 3H) |
| 1.081 | 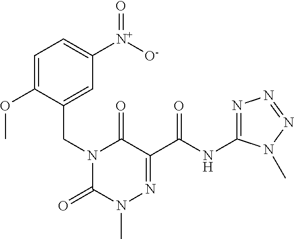 [SMILES: COc1ccc([N+](=O)[O-])cc1Cn1c(=O)c(C(=O)Nc2nnnn2C)nn(C)c1=O] | d3-MeCN: 10.75(b s, 1H), 8.23(dd, 1H), 8.08(d, 1H), 7.15(d, 1H), 5.18(s, 2H), 3.99(s, 3H), 3.94(s, 3H), 3.76(s, 3H) |
| 1.082 | 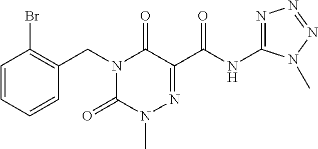 [SMILES: Cn1nnnc1NC(=O)c1nn(C)c(=O)n(Cc2ccccc2Br)c1=O] | d3-MeCN: 10.70(b s, 1H), 7.69(m, 1H), 7.38-7.18(m, 3H), 5.22(s, 2H), 3.95(s, 3H), 3.79(s, 3H) |
| 1.083 | 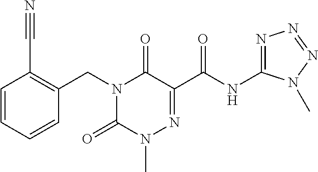 [SMILES: Cn1nnnc1NC(=O)c1nn(C)c(=O)n(Cc2ccccc2C#N)c1=O] | d3-MeCN: 10.68(b s, 1H), 7.79(d, 1H), 7.65(t, 1H), 7.52-7.45(m, 2H), 5.33(s, 2H), 3.95(s, 3H), 3.77(s, 3H) |
| 1.084 | 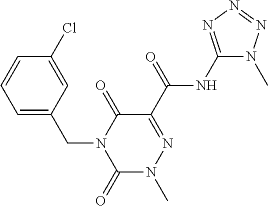 [SMILES: Cn1nnnc1NC(=O)c1nn(C)c(=O)n(Cc2cccc(Cl)c2)c1=O] | d3-MeCN: 10.78(b s, 1H), 7.48(s, 1H), 7.44-7.32(m, 3H), 5.12(s, 2H), 3.95(s, 3H), 3.74(s, 3H) |
| 1.085 | 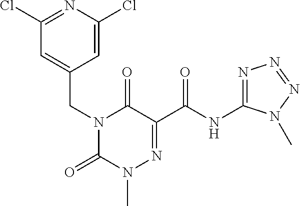 [SMILES: Cn1nnnc1NC(=O)c1nn(C)c(=O)n(Cc2cc(Cl)nc(Cl)c2)c1=O] | d3-MeCN: 10.62(b s, 1H), 7.41(s, 2H), 5.13(s, 2H), 3.96(s, 1H), 3.76(s, 3H) |
| 1.086 | 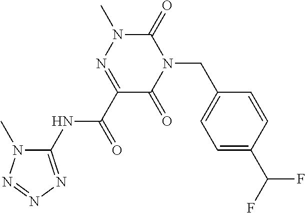 [SMILES: Cn1nnnc1NC(=O)c1nn(C)c(=O)n(Cc2ccc(C(F)F)cc2)c1=O] | d3-MeCN: 10.20(b, 1H), 7.60-7.51(m, 4H), 7.79(t, 1H), 5.18(s, 2H), 3.95(s, 3H), 3.74(s, 3H) |
| 1.087 | 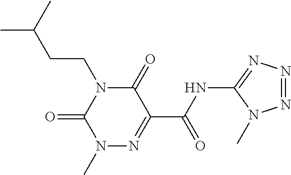 [SMILES: CC(C)CCn1c(=O)c(C(=O)Nc2nnnn2C)nn(C)c1=O] | CDCl3: 11.35(b s, 1H), 4.07(s, 3H), 3.88(s, 3H), 1.73-1.60(m, 3H), 1.58(m, 2H), 0.99(d, 6H) |
| 1.088 | 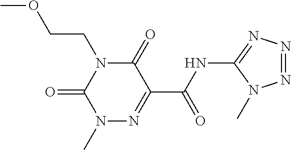 [SMILES: COCCn1c(=O)c(C(=O)Nc2nnnn2C)nn(C)c1=O] | CDCl3: 11.29(b s, 1H), 4.29(t, 2H), 4.07(s, 3H), 3.87(s, 3H), 3.72(t, 2H), 3.36(s, 3H) |
| 1.089 | 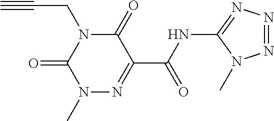 [SMILES: C#CCn1c(=O)c(C(=O)Nc2nnnn2C)nn(C)c1=O] | d3-MeCN: 10.68(b, 1H), 4.72(d, 2H), 3.96(s, 3H), 3.77(s, 3H), 2.58(t, 1H) |
| 1.090 | 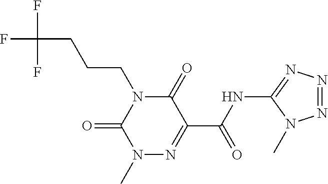 [SMILES: Cn1nnnc1NC(=O)c1nn(C)c(=O)n(CCCC(F)(F)F)c1=O] | CDCl3: 11.25(b, 1H), 4.11(t, 2H), 4.06(s, 3H), 3.87(s, 3H), 2.22(m, 2H), 1.98(m, 2H) |
| 1.091 | 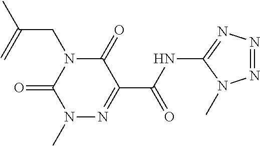 [SMILES: C=C(C)Cn1c(=O)c(C(=O)Nc2nnnn2C)nn(C)c1=O] | CDCl3: 11.27(b, 1H), 5.01(s, 1H), 4.83(s, 1H), 4.59(s, 2H), 4.07(s, 3H), 4.89(s, 3H), 1.82(s, 3H) |
| 1.092 | 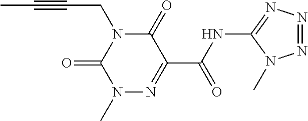 [SMILES: CC#CCn1c(=O)c(C(=O)Nc2nnnn2C)nn(C)c1=O] | CDCl3: 11.21(s, 1H), 4.75(q, 2H), 4.08(s, 3H), 3.89(s, 3H), 1.81(t, 3H), |
| 1.093 | 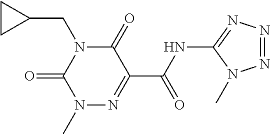 [SMILES: Cn1nnnc1NC(=O)c1nn(C)c(=O)n(CC2CC2)c1=O] | CDCl3: 11.36(b s, 1H), 4.07(s, 3H), 3.93(d, 2H, 3.88(s, 3H), 1.28(m, 1H), 0.61-0.56(m, 2H), 0.48- 0.44(m, 2H) |
| 1.094 | 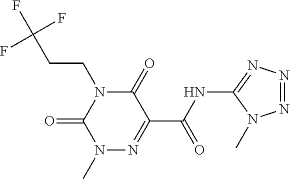 [SMILES: Cn1nnnc1NC(=O)c1nn(C)c(=O)n(CCC(F)(F)F)c1=O] | CDCl3: 11.11 (b s, 1H), 4.34(t, 2H), 4.08(s, 3H), 3.88(s, 3H), 2.63-2.53(m, 2H) |
| 1.095 | 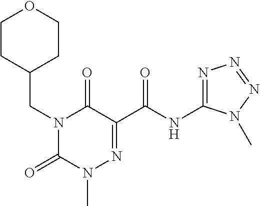 [SMILES: Cn1nnnc1NC(=O)c1nn(C)c(=O)n(CC2CCOCC2)c1=O] | CDCl3: 11.27(s, 1H), 4.07(s, 3H), 4.07(s, 3H), 4.03-3.95(m, 2H), 4.96(d, 2H), 3.36(dt, 2H), 2.10(m, 1H), 1.54-1.42(m, 4H) |
| 1.096 | 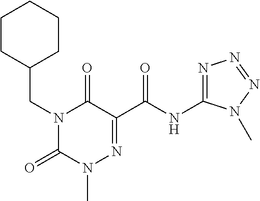 [SMILES: Cn1nnnc1NC(=O)c1nn(C)c(=O)n(CC2CCCCC2)c1=O] | CDCl3: 11.35(s, 1H), 4.07(s, 3H), 4.88(s, 3H), 4.87(t, 2H), 1.83(m, 1H), 1.28-1.02(m, 10H) |
| 1.097 | 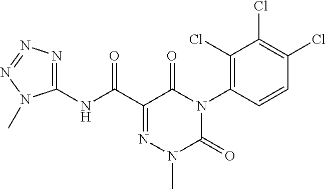 [SMILES: Cn1nnnc1NC(=O)c1nn(C)c(=O)n(-c2ccc(Cl)c(Cl)c2Cl)c1=O] | |
| 1.098 | 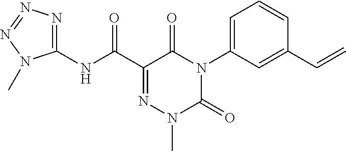 [SMILES: C=Cc1cccc(-n2c(=O)c(C(=O)Nc3nnnn3C)nn(C)c2=O)c1] | |
| 1.099 | 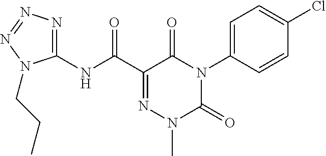 [SMILES: CCCn1nnnc1NC(=O)c1nn(C)c(=O)n(-c2ccc(Cl)cc2)c1=O] | |
| 1.100 | 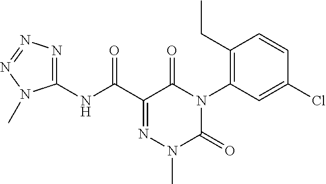 [SMILES: CCc1ccc(Cl)cc1-n1c(=O)c(C(=O)Nc2nnnn2C)nn(C)c1=O] | |
| 1.101 | 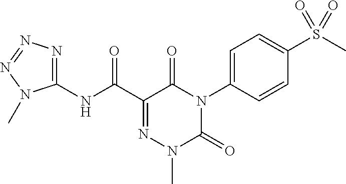 [SMILES: Cn1nnnc1NC(=O)c1nn(C)c(=O)n(-c2ccc(S(C)(=O)=O)cc2)c1=O] | |
| 1.102 | 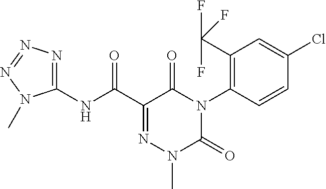 [SMILES: Cn1nnnc1NC(=O)c1nn(C)c(=O)n(-c2ccc(Cl)cc2C(F)(F)F)c1=O] | |
| 1.103 | 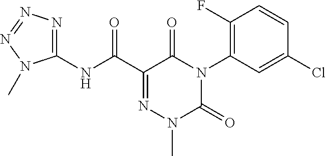 [SMILES: Cn1nnnc1NC(=O)c1nn(C)c(=O)n(-c2cc(Cl)ccc2F)c1=O] | |
| 1.104 | 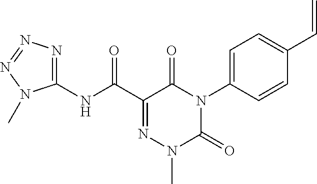 [SMILES: C=Cc1ccc(-n2c(=O)c(C(=O)Nc3nnnn3C)nn(C)c2=O)cc1] | |
| 1.105 | 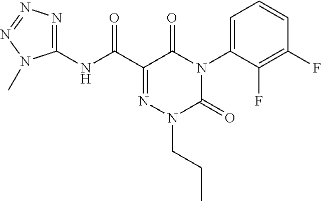 [SMILES: CCCn1nc(C(=O)Nc2nnnn2C)c(=O)n(-c2cccc(F)c2F)c1=O] | |
| 1.106 | 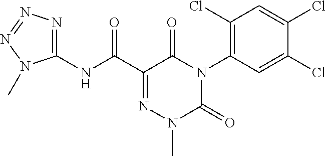 [SMILES: Cn1nnnc1NC(=O)c1nn(C)c(=O)n(-c2cc(Cl)c(Cl)cc2Cl)c1=O] | |
| 1.107 | 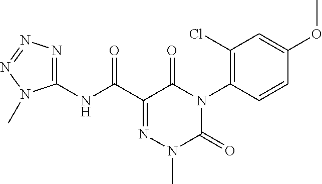 [SMILES: COc1ccc(-n2c(=O)c(C(=O)Nc3nnnn3C)nn(C)c2=O)c(Cl)c1] | |
| 1.108 | 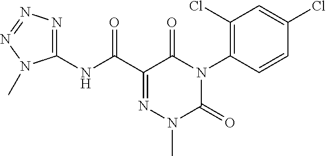 [SMILES: Cn1nnnc1NC(=O)c1nn(C)c(=O)n(-c2ccc(Cl)cc2Cl)c1=O] | |
| 1.109 | 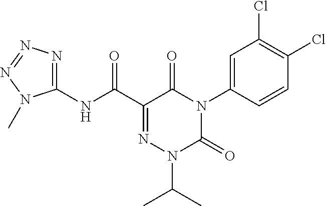 [SMILES: CC(C)n1nc(C(=O)Nc2nnnn2C)c(=O)n(-c2ccc(Cl)c(Cl)c2)c1=O] | |
| 1.110 | 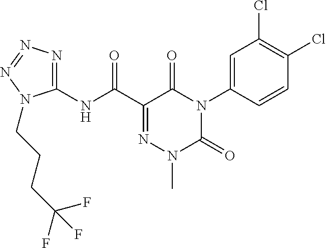 [SMILES: Cn1nc(C(=O)Nc2nnnn2CCCC(F)(F)F)c(=O)n(-c2ccc(Cl)c(Cl)c2)c1=O] | |
| 1.111 | 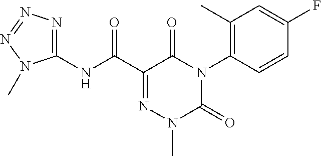 [SMILES: Cc1cc(F)ccc1-n1c(=O)c(C(=O)Nc2nnnn2C)nn(C)c1=O] | |
| 1.112 | 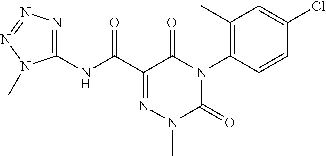 [SMILES: Cc1cc(Cl)ccc1-n1c(=O)c(C(=O)Nc2nnnn2C)nn(C)c1=O] | |
| 1.113 | 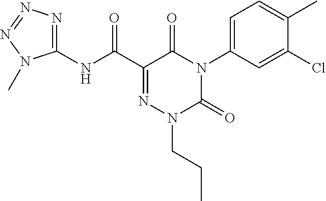 [SMILES: CCCn1nc(C(=O)Nc2nnnn2C)c(=O)n(-c2ccc(C)c(Cl)c2)c1=O] | |
| 1.114 | 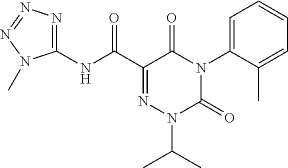 [SMILES: Cc1ccccc1-n1c(=O)c(C(=O)Nc2nnnn2C)nn(C(C)C)c1=O] | |
| 1.115 | 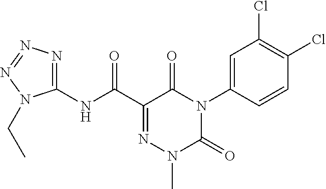 [SMILES: CCn1nnnc1NC(=O)c1nn(C)c(=O)n(-c2ccc(Cl)c(Cl)c2)c1=O] | |
| 1.116 | 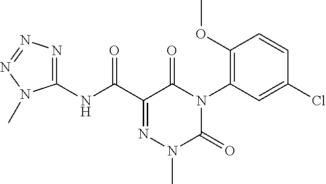 [SMILES: COc1ccc(Cl)cc1-n1c(=O)c(C(=O)Nc2nnnn2C)nn(C)c1=O] | |
| 1.117 | 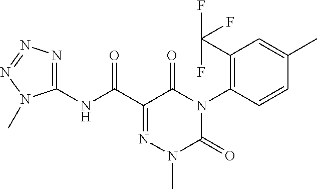 [SMILES: Cc1ccc(-n2c(=O)c(C(=O)Nc3nnnn3C)nn(C)c2=O)c(C(F)(F)F)c1] | |
| 1.118 | 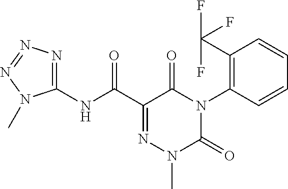 [SMILES: Cn1nnnc1NC(=O)c1nn(C)c(=O)n(-c2ccccc2C(F)(F)F)c1=O] | |
| 1.119 | 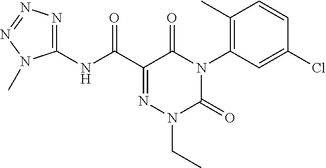 [SMILES: CCn1nc(C(=O)Nc2nnnn2C)c(=O)n(-c2cc(Cl)ccc2C)c1=O] | |
| 1.120 | 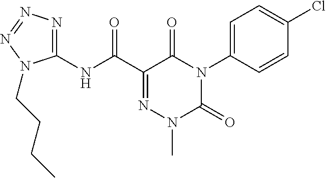 [SMILES: CCCCn1nnnc1NC(=O)c1nn(C)c(=O)n(-c2ccc(Cl)cc2)c1=O] | |
| 1.121 | 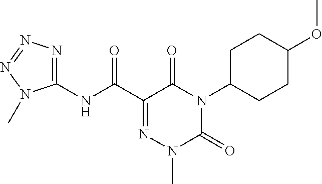 [SMILES: COC1CCC(n2c(=O)c(C(=O)Nc3nnnn3C)nn(C)c2=O)CC1] | |
| 1.122 | 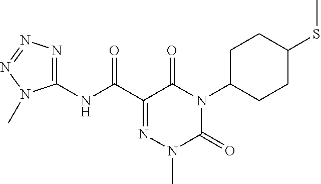 [SMILES: CSC1CCC(n2c(=O)c(C(=O)Nc3nnnn3C)nn(C)c2=O)CC1] | |
| 1.123 | 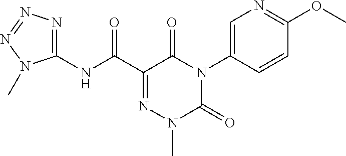 [SMILES: COc1ccc(-n2c(=O)c(C(=O)Nc3nnnn3C)nn(C)c2=O)cn1] | d6-DMSO: inter alia 8.09(d, 1H), 7.66(dd, 1H), 6.95(d, 1H), 3.89(s, 3H), 3.61(s, 3), 3.51(s, 3H) |
| 1.124 | 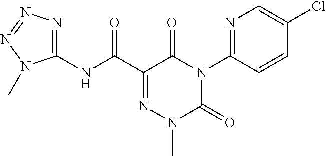 [SMILES: Cn1nnnc1NC(=O)c1nn(C)c(=O)n(-c2ccc(Cl)cn2)c1=O] | |
| 1.125 | 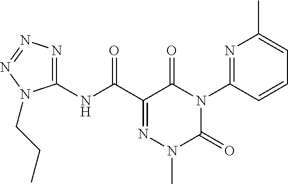 [SMILES: CCCn1nnnc1NC(=O)c1nn(C)c(=O)n(-c2cccc(C)n2)c1=O] | |
| 1.126 | 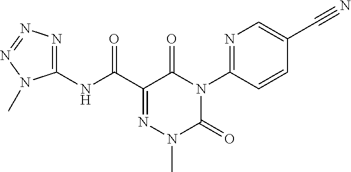 [SMILES: Cn1nnnc1NC(=O)c1nn(C)c(=O)n(-c2ccc(C#N)cn2)c1=O] | |
| 1.127 | 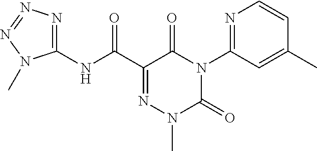 [SMILES: Cc1ccnc(-n2c(=O)c(C(=O)Nc3nnnn3C)nn(C)c2=O)c1] | |
| 1.128 | 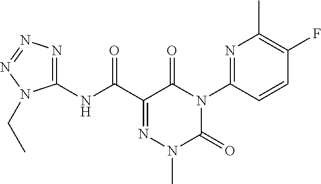 [SMILES: CCn1nnnc1NC(=O)c1nn(C)c(=O)n(-c2ccc(F)c(C)n2)c1=O] | |
| 1.129 | 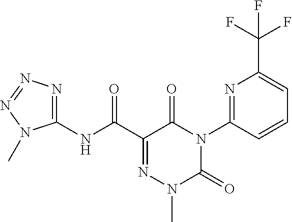 [SMILES: Cn1nnnc1NC(=O)c1nn(C)c(=O)n(-c2cccc(C(F)(F)F)n2)c1=O] | |
| 1.130 | 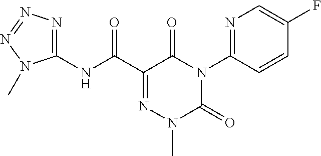 [SMILES: Cn1nnnc1NC(=O)c1nn(C)c(=O)n(-c2ccc(F)cn2)c1=O] | |
| 1.131 | 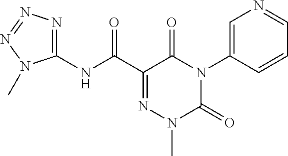 [SMILES: Cn1nnnc1NC(=O)c1nn(C)c(=O)n(-c2cccnc2)c1=O] | |
| 1.132 | 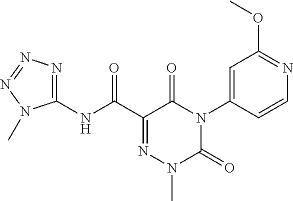 [SMILES: COc1cc(-n2c(=O)c(C(=O)Nc3nnnn3C)nn(C)c2=O)ccn1] | |

TABLE 2
Examples of herbicidal compounds of the present invention.

| | | |
|---|---|---|
| 2.001 | 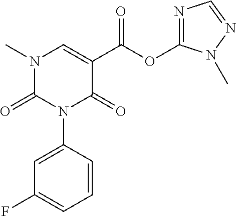 [SMILES: Cn1ncnc1OC(=O)c1cn(C)c(=O)n(-c2cccc(F)c2)c1=O] | d6-DMSO 10.68(s, 1H), 8.90(s, 1H), 7.86(s, 1H), 7.60- 7.54(m, 1H)m 7.37- 7.27(m, 2H), 7.22(d, 1H), 3.67(s, 3H), 3.52(s, 3H) |
| 2.002 | 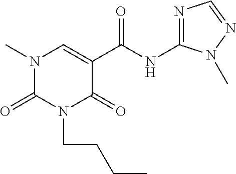 [SMILES: CCCCn1c(=O)c(C(=O)Nc2ncnn2C)cn(C)c1=O] | |
| 2.003 | 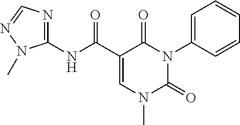 [SMILES: Cn1ncnc1NC(=O)c1cn(C)c(=O)n(-c2ccccc2)c1=O] | d6-DMSO: 10.74(bs, 1H), 8.89(s, 1H), 7.85(s, 1H), 7.53- 7.44(m, 3H), 7.35-7.30 (m, 2H), 3.68(s, 3H), 3.52(s, 3H) |
| 2.004 | 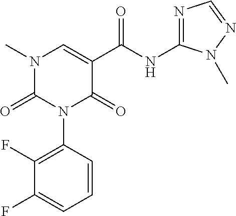 [SMILES: Cn1ncnc1NC(=O)c1cn(C)c(=O)n(-c2cccc(F)c2F)c1=O] | |

TABLE 3
Examples of herbicidal compounds of the present invention.

| | | |
|---|---|---|
| 3.001 | 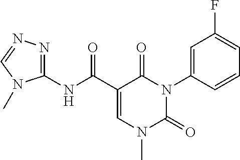 [SMILES: Cn1cnnc1NC(=O)c1cn(C)c(=O)n(-c2cccc(F)c2)c1=O] | |
| 3.002 | 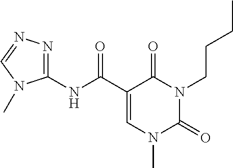 [SMILES: CCCCn1c(=O)c(C(=O)Nc2nncn2C)cn(C)c1=O] | |
| 3.003 | 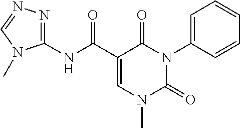 [SMILES: Cn1cnnc1NC(=O)c1cn(C)c(=O)n(-c2ccccc2)c1=O] | d6-DMSO: 10.66(b s, 1H), 8.88(s, 1H), 8.48(s, 1H), 7.56- 7.44(m, 3H), 7.36- 7.30(d, 2H), 3.52(s, 3H), 3.48(s, 3H) |
| 3.004 | 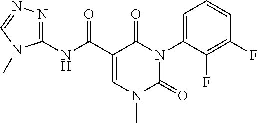 [SMILES: Cn1cnnc1NC(=O)c1cn(C)c(=O)n(-c2cccc(F)c2F)c1=O] | |

TABLE 4
Examples of herbicidal compounds of the present invention.

| | | |
|---|---|---|
| 4.001 | 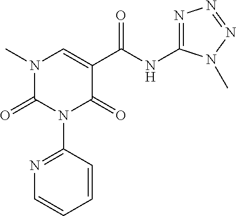 [SMILES: Cn1nnnc1NC(=O)c1cn(C)c(=O)n(-c2ccccn2)c1=O] | |
| 4.002 | 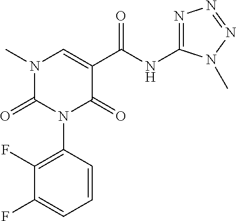 [SMILES: Cn1nnnc1NC(=O)c1cn(C)c(=O)n(-c2cccc(F)c2F)c1=O] | |
| 4.003 | 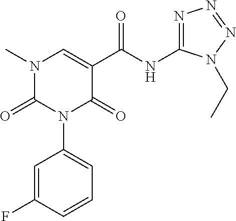 [SMILES: CCn1nnnc1NC(=O)c1cn(C)c(=O)n(-c2cccc(F)c2)c1=O] | d6-DMSO 10.91(s, 1H), 8.94(s, 1H), 7.62- 7.54(q, 1H), 7.38-7.28 (m, 2H), 7.23(d, 1H), 4.23(q, 2H), 3.53(s, 3H), 1.42(t, 3H) |
| 4.004 | 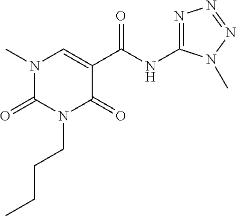 [SMILES: CCCCn1c(=O)c(C(=O)Nc2nnnn2C)cn(C)c1=O] | d6-DMSO: 11.19(1H, s), 8.79(1H, s), 3.91 (3H, s), 3.89(2H, m), 3.49(3H, s), 1.56(2H, m), 1.32(2H, m), 0.91 (3H, t). |
| 4.005 | 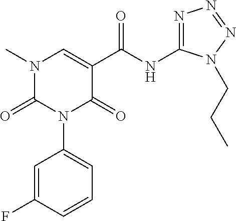 [SMILES: CCCn1nnnc1NC(=O)c1cn(C)c(=O)n(-c2cccc(F)c2)c1=O] | (CDCl3) 11.09(s, 1H), 8.64(s, 1H), 7.57-7.50 (m, 1H), 7.25-7.21 (m, 1H), 7.05(d, 1H), 7.02- 6.98(dt, 1H), 4.25(t, 2H), 3.65(s, 3H), 2.00- 1.90(m, 2H), 0.95(t, 3H) |

TABLE 5
Examples of herbicidal compounds of the present invention.

| | | |
|---|---|---|
| 5.001 | 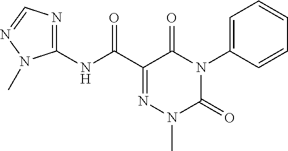 [SMILES: Cn1ncnc1NC(=O)c1nn(C)c(=O)n(-c2ccccc2)c1=O] | d6-DMSO: 10.95(bs, 1H), 7.90(bs, 1H), 7.55-7.64(m, 3H), 7.33-7.22(m, 2H), 3.69(s, 3H), 3.67(s, 3H) |
| 5.002 | 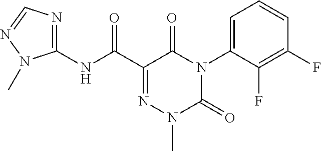 [SMILES: Cn1ncnc1NC(=O)c1nn(C)c(=O)n(-c2cccc(F)c2F)c1=O] | d6-DMSO: 10.99(bs, 1H), 7.91(bs, 1H), 7.68-7.62(m, 1H), 7.44-7.35(m, 2H), 3.69(s, 3H), 3.62(s, 3H) |
| 5.003 | 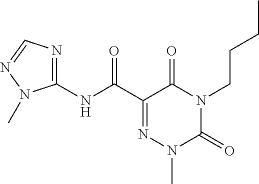 [SMILES: CCCCn1c(=O)c(C(=O)Nc2ncnn2C)nn(C)c1=O] | d6-DMSO: 11.00(bs, 1H), 7.90(bs, 1H), 3.84(m, 2H), 3.69(s, 3H), 3.62(s, 3H), 1.55(m, 2H), 1.31(m, 2H), 0.90(t, 3H) |

TABLE 6
Examples of herbicidal compounds of the present invention.

| | | |
|---|---|---|
| 6.001 | 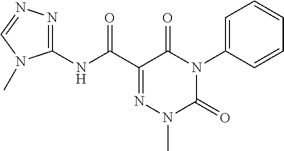 [SMILES: Cn1cnnc1NC(=O)c1nn(C)c(=O)n(-c2ccccc2)c1=O] | d6-DMSO: 10.79(b, 1H), 8.48(bs, 1H), 7.52-7.47(m, 3H), 7.35-7.33(m, 2H), 3.57(bs, 3H), 3.32(s, 3H) |
| 6.002 | 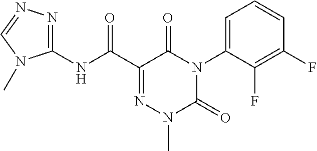 [SMILES: Cn1cnnc1NC(=O)c1nn(C)c(=O)n(-c2cccc(F)c2F)c1=O] | d6-DMSO: 10.84(bs, 1H), 8.45(bs, 1H), 7.64(m, 1H), 7.43-7.31(m, 2H), 3.53(bs, 3H), 3.31(s, 3H) |
| 6.003 | 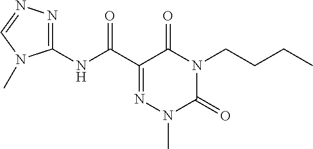 [SMILES: CCCCn1c(=O)c(C(=O)Nc2nncn2C)nn(C)c1=O] | d6-DMSO: 10.83(bs, 1H), 8.49(b, 1H), 3.81(b, 2H), 3.63(bs, 3H), 3.49(bs, 3H), 1.55(b, 2H), 1.32(b, 2H), 0.90(t, 3H) |

TABLE 7
Examples of compounds of the present invention.

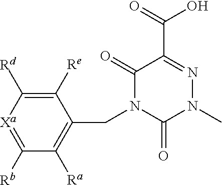

| Compound | X^{a} | R^{a} | R^{b} | R^{c} | R^{d} | R^{e} | NMR/MS |
|---|---|---|---|---|---|---|---|
| 7.001 | C—R^{c} | H | H | —CN | H | H | 287.12 (M + H) |
| 7.002 | C—R^{c} | H | H | Me | H | H | 276.14 (M + H) |
| 7.003 | C—R^{c} | H | Me | H | H | H | 276.15 (M + H) |
| 7.004 | C—R^{c} | Me | H | H | H | H | 276.13 (M + H) |
| 7.005 | C—R^{c} | H | —CN | H | H | H | 287.15 (M + H) |
| 7.006 | C—R^{c} | F | H | F | H | H | 298.11 (M + H) |
| 7.007 | C—R^{c} | F | H | F | H | F | 316.12 (M + H) |
| 7.008 | C—R^{c} | H | H | —CF₃ | H | H | 330.13 (M + H) |
| 7.009 | C—R^{c} | F | H | H | H | H | 280.11 (M + H) |
| 7.010 | C—R^{c} | Cl | H | H | H | H | 296.10 (Cl 35 isotope) (M + H) |
| 7.011 | C—R^{c} | NO₂ | H | H | H | H | 307.12 (M + H) |
| 7.012 | C—R^{c} | F | H | H | F | H | 298.11 (M + H) |
| 7.013 | C—R^{c} | —CF₃ | H | H | H | H | 330.13 (M + H) |
| 7.014 | C—R^{c} | H | F | H | H | H | 280.11 (M + H) |
| 7.015 | C—R^{c} | H | F | F | H | H | 298.11 (M + H) |
| 7.016 | C—R^{c} | F | H | H | H | F | 298.18 (M + H) |
| 7.017 | C—R^{c} | H | H | —O—CHF₂ | H | H | 328.14 (M + H) |
| 7.018 | C—R^{c} | F | F | H | H | H | 298.11 (M + H) |
| 7.019 | C—R^{c} | H | Cl | Cl | H | H | 330.06 (Cl 35 isotope) (M + H) |
| 7.020 | C—R^{c} | Cl | H | F | H | H | 314.10 (Cl 35 isotope) (M + H) |
| 7.021 | N | H | Cl | — | MeO— | H | 327.11 (Cl 35 isotope) (M + H) |
| 7.022 | C—R^{c} | H | H | NO₂ | H | H | 307.13 (M + H) |
| 7.023 | C—R^{c} | Cl | H | H | H | Cl | 330.06 (Cl 35 isotope) (M + H) |
| 7.024 | C—R^{c} | H | H | F | H | H | 280.13 (M + H) |
| 7.025 | C—R^{c} | H | Me | H | Me | H | 290.17 (M + H) |
| 7.026 | C—R^{c} | H | Me | F | H | H | 294.14 (M + H) |
| 7.027 | C—R^{c} | —CF₃ | F | H | H | H | 348.11 (M + H) |
| 7.028 | C—R^{c} | Cl | MeO— | H | H | F | 344.09 (Cl 35 isotope) (M + H) |
| 7.029 | C—R^{c} | H | Me | F | Me | H | 308.18 (M + H) |
| 7.030 | C—R^{c} | H | —CHF₂ | H | H | H | 312.13 (M + H) |
| 7.031 | C—R^{c} | H | H | —S—CF₃ | H | H | 362.10 (M + H) |
| 7.032 | C—R^{c} | MeO— | H | H | —NO₂ | H | 337.13 (M + H) |
| 7.033 | C—R^{c} | Br | H | H | H | H | 342.06 (Br 81 isotope) (M + H) |
| 7.034 | C—R^{c} | —CN | H | H | H | H | 287.11 (M + H) |
| 7.035 | C—R^{c} | H | Cl | H | H | H | 296.09 (Cl 35 isotope) (M + H) |
| 7.036 | N | H | Cl | — | Cl | H | 331.07 (Cl 35 isotope) (M + H) |
| 7.037 | C—R^{c} | H | H | —CHF₂ | H | H | 312.13 (M + H) |
| 7.038 | C—R^{c} | H | H | H | H | H | 1Hnmr(CDCl3): 12.30(broad, 1H), |
| | | | | | | | 7.54-7.49(m, 2H), 7.38-7.34(m, |
| | | | | | | | 3H), 5.19(s, 2H), 3.87(s, 3H) |

TABLE 8
Examples of compounds of the present invention.

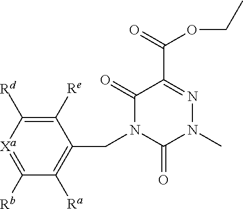

| Compound | X^{a} | R^{a} | R^{b} | R^{c} | R^{d} | R^{e} | NMR/MS |
|---|---|---|---|---|---|---|---|
| 8.001 | C—R^{c} | H | H | —CN | H | H | 315.24 (M + H) |
| 8.002 | C—R^{c} | H | H | Me | H | H | 304.24 (M + H) |
| 8.003 | C—R^{c} | H | Me | H | H | H | 304.24 (M + H) |
| 8.004 | C—R^{c} | Me | H | H | H | H | 304.24 (M + H) |
| 8.005 | C—R^{c} | H | —CN | H | H | H | 315.22 (M + H) |
| 8.006 | C—R^{c} | F | H | F | H | H | 326.21 (M + H) |
| 8.007 | C—R^{c} | F | H | F | H | F | 344.2 (M + H) |
| 8.008 | C—R^{c} | H | H | —CF₃ | H | H | 358.21 (M + H) |
| 8.009 | C—R^{c} | F | H | H | H | H | 308.22 (M + H) |
| 8.010 | C—R^{c} | Cl | H | H | H | H | 324.18 (Cl 35 isotope) (M + H) |
| 8.011 | C—R^{c} | NO₂ | H | H | H | H | 335.21 (M + H) |
| 8.012 | C—R^{c} | F | H | H | F | H | 326.2 (M + H) |
| 8.013 | C—R^{c} | —CF₃ | H | H | H | H | 358.2 (M + H) |
| 8.014 | C—R^{c} | H | F | H | H | H | 308.2 (M + H) |
| 8.015 | C—R^{c} | H | F | F | H | H | not observed |
| 8.016 | C—R^{c} | F | H | H | H | F | 326.2 (M + H) |
| 8.017 | C—R^{c} | H | H | —O—CHF₂ | H | H | 365.22 (M + H) |
| 8.018 | C—R^{c} | F | F | H | H | H | 326.2 (M + H) |
| 8.019 | C—R^{c} | H | Cl | Cl | H | H | 358.15 (Cl 35 isotope) (M + H) |
| 8.020 | C—R^{c} | Cl | H | F | H | H | 342.18 (Cl 35 isotope) (M + H) |
| 8.021 | N | H | Cl | — | MeO— | H | 355.19 (Cl 35 isotope) (M + H) |
| 8.022 | C—R^{c} | H | H | NO₂ | H | H | 335.21 (M + H) |
| 8.023 | C—R^{c} | Cl | H | H | H | Cl | 358.15 (Cl 35 isotope) (M + H) |
| 8.024 | C—R^{c} | H | H | F | H | H | 308.2 (M + H) |
| 8.025 | C—R^{c} | H | Me | H | Me | H | 318.26 (M + H) |
| 8.026 | C—R^{c} | H | Me | F | H | H | 322.24 (M + H) |
| 8.027 | C—R^{c} | —CF₃ | F | H | H | H | 376.20 (M + H) |
| 8.028 | C—R^{c} | Cl | MeO— | H | H | F | 372.17 (M + H) |
| 8.029 | C—R^{c} | H | Me | F | Me | H | 336.25 (M + H) |
| 8.030 | C—R^{c} | H | —CHF₂ | H | H | H | 340.22 (M + H) |
| 8.031 | C—R^{c} | H | H | —S—CF₃ | H | H | 390.20 (M + H) |
| 8.032 | C—R^{c} | MeO— | H | H | —NO₂ | H | 365.22 (M + H) |
| 8.033 | C—R^{c} | Br | H | H | H | H | 368.13 (Br 79 isotope) (M + H) |
| 8.034 | C—R^{c} | —CN | H | H | H | H | 315.21 (M + H) |
| 8.035 | C—R^{c} | H | Cl | H | H | H | 324.18 (Cl 35 isotope) (M + H) |
| 8.036 | N | H | Cl | — | Cl | H | not observed |
| 8.037 | C—R^{c} | H | H | CHF₂ | H | H | 340.22 (M + H) |
| 8.038 | C—R^{c} | H | H | H | H | H | 1H nmr(CDCl3): 7.52(d, 2H), |
| | | | | | | | 7.35-7.30(m, 3H), 5.12(s, 2H), |
| | | | | | | | 4.42(q, 2H), 3.72(s, 3H), 1.39(t, |
| | | | | | | | 3H) |

### Biological Examples

Seeds of a variety of test species are sown in standard soil in pots (Alopecurus myosuroides (ALOMY), Setaria faberi (SETFA), Echinochloa crus-galli (ECHCG), Solanum nigrum (SOLNI), Amaranthus retoflexus (AMARE), Ipomoea hederacea (IPOHE)). After cultivation for one day (pre-emergence) or after 8 days cultivation (post-emergence) under controlled conditions in a glasshouse (at 24/16° C., day/night; 14 hours light; 65% humidity), the plants are sprayed with an aqueous spray solution derived from the formulation of the technical active ingredient in acetone / water (50:50) solution containing 0.5% Tween 20 (polyoxyethelyene sorbitan monolaurate,

CAS RN 9005-64-5). Compounds are applied at 1000 g/h. The test plants are then grown in a glasshouse under controlled conditions in a glasshouse (at 24/16° C., day/night; 14 hours light; 65% humidity) and watered twice daily. After 13 days for pre and post-emergence, the test is evaluated for the percentage damage caused to the plant. The biological activities are shown in the following table on a five point scale (5=80-100%; 4=60-79%; 3=40-59%; 2=20-39%; 1=0-19%).

| | POST Application | | | | | | PRE Application | | | | | |
|---|---|---|---|---|---|---|---|---|---|---|---|---|
| Compound | SOLNI | AMARE | SETFA | ALOMY | ECHCG | IPOHE | SOLNI | AMARE | SETFA | ALOMY | ECHCG | IPOHE |
| 1.001 | 5 | 5 | 5 | 5 | 5 | 5 | 5 | 5 | 5 | 5 | 5 | 5 |
| 1.002 | 5 | 5 | 5 | 5 | 5 | 5 | 5 | 5 | 5 | 5 | 5 | 5 |
| 1.003 | 5 | 5 | 5 | 5 | 5 | 5 | 5 | 5 | 5 | 5 | 5 | 5 |
| 1.004 | 5 | 5 | 4 | 4 | 4 | 5 | 5 | 5 | 2 | 3 | 4 | 4 |
| 1.005 | 5 | 5 | 5 | 5 | 5 | 5 | 5 | 5 | 5 | 5 | 5 | 4 |
| 1.006 | 5 | 5 | 5 | 5 | 5 | 5 | 5 | 5 | 5 | 5 | 5 | 5 |
| 1.007 | 5 | 5 | 5 | 5 | 5 | 5 | 5 | 5 | 1 | 2 | 5 | 5 |
| 1.008 | 5 | 5 | 5 | 5 | 5 | 5 | 5 | 5 | 5 | 5 | 5 | 5 |
| 1.009 | 5 | 5 | 5 | 5 | 5 | 5 | 5 | 5 | 1 | 2 | 2 | 4 |
| 1.010 | 5 | 5 | 5 | 5 | 5 | 5 | 5 | 4 | 5 | 5 | 5 | 5 |
| 1.011 | 5 | 5 | 5 | 5 | 5 | 5 | 5 | 5 | 4 | 4 | 5 | 5 |
| 1.012 | 5 | 5 | 5 | 5 | 5 | 4 | 5 | 4 | 1 | 4 | 3 | 5 |
| 1.013 | 5 | 2 | 3 | 5 | 1 | 4 | 3 | 2 | 1 | 1 | 1 | 2 |
| 1.014 | 5 | 5 | 5 | 2 | 4 | 5 | 5 | 5 | 1 | 3 | 5 | 5 |
| 1.015 | 5 | 5 | 5 | 5 | 5 | 5 | 5 | 5 | 5 | 5 | 5 | 5 |
| 1.016 | 5 | 5 | 5 | 5 | 5 | 5 | 5 | 5 | 5 | 5 | 5 | 5 |
| 1.017 | 5 | 5 | 5 | 5 | 5 | 5 | 5 | 5 | 5 | 5 | 5 | 5 |
| 1.018 | 5 | 5 | 5 | 5 | 5 | 5 | 5 | 5 | 5 | 5 | 5 | 5 |
| 1.019 | 5 | 5 | 5 | 5 | 5 | 5 | 5 | 5 | 5 | 5 | 5 | 5 |
| 1.020 | 5 | 5 | 5 | 5 | 5 | 5 | 5 | 5 | 5 | 5 | 5 | 5 |
| 1.021 | 5 | 5 | 5 | 5 | 5 | 5 | 5 | 5 | 5 | 5 | 5 | 5 |
| 1.022 | 5 | 5 | 4 | 4 | 5 | 5 | 5 | 5 | 3 | 5 | 5 | 4 |
| 1.023 | 5 | 5 | 5 | 5 | 5 | 5 | 5 | 5 | 5 | 5 | 5 | 5 |
| 1.024 | 5 | 5 | 5 | 5 | 5 | 5 | 5 | 5 | 5 | 5 | 5 | 5 |
| 1.025 | 5 | 5 | 5 | 5 | 5 | 5 | 5 | 5 | 5 | 5 | 5 | 5 |
| 1.026 | 5 | 5 | 5 | 5 | 5 | 5 | 5 | 5 | 5 | 5 | 5 | 5 |
| 1.027 | 5 | 5 | 5 | 5 | 5 | 5 | 5 | 5 | 5 | 5 | 5 | 5 |
| 1.028 | 5 | 5 | 5 | 5 | 5 | 5 | 5 | 5 | 5 | 5 | 5 | 5 |
| 1.029 | 5 | 5 | 5 | 5 | 5 | 5 | 5 | 5 | 5 | 5 | 5 | 5 |
| 1.031 | 5 | 5 | 5 | 4 | 5 | 2 | 5 | 5 | 4 | 3 | 4 | 4 |
| 1.032 | 5 | 5 | 4 | 5 | 5 | 5 | 5 | 5 | 5 | 5 | 5 | 4 |
| 1.033 | 5 | 5 | 5 | 5 | 5 | 5 | 5 | 5 | 5 | 5 | 5 | 5 |
| 1.034 | 5 | 5 | 5 | 5 | 5 | 5 | 5 | 5 | 5 | 5 | 5 | 5 |
| 1.035 | 5 | 5 | 5 | 5 | 5 | 5 | 5 | 5 | 4 | 5 | 5 | 5 |
| 1.037 | 5 | 5 | 5 | 5 | 5 | 5 | 5 | 5 | 5 | 5 | 5 | 5 |
| 1.038 | 5 | 5 | 5 | 5 | 5 | 5 | 5 | 5 | 5 | 5 | 5 | 5 |
| 1.039 | 5 | 5 | 5 | 5 | 5 | 5 | 5 | 5 | 5 | 5 | 5 | 5 |
| 1.040 | 5 | 5 | 5 | 4 | 5 | 4 | 5 | 5 | 5 | 5 | 5 | 3 |
| 1.041 | 5 | 5 | 5 | 5 | 5 | 5 | 5 | 5 | 4 | 5 | 5 | 5 |
| 1.042 | 5 | 5 | 5 | 5 | 5 | 5 | 5 | 5 | 5 | 5 | 5 | 5 |
| 1.043 | 5 | 5 | 4 | 5 | 5 | 5 | 5 | 5 | 2 | 4 | 5 | 5 |
| 1.044 | 5 | 5 | 5 | 5 | 5 | 5 | 5 | 5 | 5 | 5 | 5 | 5 |
| 1.045 | 5 | 5 | 5 | 5 | 5 | 5 | 5 | 5 | 5 | 5 | 5 | 5 |
| 1.046 | 5 | 5 | 5 | 5 | 5 | 5 | 5 | 5 | 5 | 5 | 5 | 5 |
| 1.047* | 5 | 5 | 1 | 3 | 3 | 4 | 5 | 5 | 1 | 1 | 3 | 5 |
| 1.048 | 5 | 5 | 5 | 5 | 5 | 5 | 5 | 5 | 5 | 5 | 5 | 5 |
| 1.049 | 5 | 5 | 5 | 5 | 5 | 5 | 5 | 5 | 5 | 5 | 5 | 5 |
| 1.050 | 5 | 5 | 1 | 5 | 5 | 5 | 5 | 5 | 2 | 5 | 5 | 5 |
| 1.051 | 5 | 5 | 2 | 5 | 5 | 5 | 5 | 5 | 1 | 5 | 5 | 5 |
| 1.052 | 5 | 5 | 1 | 5 | 5 | 5 | 5 | 5 | 1 | 5 | 5 | 2 |
| 1.053 | 5 | 5 | 3 | 5 | 4 | 5 | 5 | 5 | 3 | 5 | 5 | 5 |
| 1.054 | 5 | 5 | 4 | 5 | 5 | 5 | 5 | 5 | 4 | 5 | 5 | 5 |
| 1.055 | 5 | 5 | 1 | 5 | 4 | 5 | 5 | 5 | 1 | 5 | 5 | 5 |
| 1.056 | 5 | 5 | 4 | 5 | 4 | 5 | 5 | 5 | 1 | 5 | 5 | 5 |
| 1.057 | 5 | 5 | 1 | 5 | 4 | 5 | 3 | 5 | 1 | 5 | 5 | 4 |
| 1.058 | 5 | 5 | 5 | 5 | 5 | 5 | 5 | 5 | 5 | 5 | 5 | 5 |
| 1.059 | 5 | 5 | 4 | 5 | 4 | 5 | 5 | 5 | 2 | 5 | 5 | 5 |
| 1.060 | 5 | 5 | 5 | 5 | 5 | 5 | 5 | 5 | 5 | 5 | 5 | 5 |
| 1.061 | 5 | 5 | 5 | 5 | 5 | 5 | 5 | 5 | 5 | 5 | 5 | 5 |
| 1.062 | 5 | 5 | 5 | 5 | 5 | 5 | 5 | 5 | 5 | 5 | 5 | 5 |
| 1.063 | 5 | 5 | 4 | 5 | 5 | 5 | 5 | 5 | 4 | 5 | 5 | 5 |
| 1.064 | 5 | 5 | 2 | 2 | 5 | 5 | 5 | 5 | 1 | 5 | 5 | 5 |
| 1.065 | 5 | 5 | 5 | 5 | 5 | 5 | 5 | 5 | 5 | 5 | 5 | 5 |
| 1.066 | 5 | 5 | 1 | 5 | 5 | 5 | 5 | 5 | 1 | 5 | 5 | 5 |
| 1.067 | 5 | 5 | 4 | 5 | 5 | 5 | 5 | 5 | 4 | 5 | 5 | 5 |
| 1.068 | 5 | 5 | 1 | 3 | 4 | 5 | 5 | 5 | 1 | 3 | 2 | 1 |
| 1.069 | 5 | 5 | 2 | 5 | 5 | 5 | 5 | 5 | 2 | 5 | 5 | 5 |
| 1.070 | 5 | 5 | 2 | 5 | 5 | 5 | 5 | 5 | 1 | 5 | 5 | 5 |
| 1.071 | 5 | 5 | 1 | 4 | 4 | 2 | 5 | 5 | 2 | 5 | 5 | 5 |
| 1.072 | 5 | 5 | 4 | 4 | 4 | 4 | 5 | 5 | 2 | 5 | 5 | 3 |
| 1.073 | 5 | 5 | 2 | 2 | 3 | 5 | 5 | 5 | 1 | 5 | 5 | 5 |
| 1.074 | 5 | 5 | 1 | 4 | 3 | 2 | 5 | 5 | 1 | 5 | 4 | 3 |
| 1.075 | 5 | 5 | 1 | 2 | 3 | 5 | 5 | 5 | 1 | 5 | 5 | 5 |
| 1.076 | 5 | 5 | 5 | 5 | 5 | 5 | 5 | 5 | 5 | 5 | 5 | 5 |
| 1.077 | 4 | 3 | 2 | 4 | 4 | 4 | 4 | 3 | 2 | 4 | 4 | 4 |
| 1.078 | 5 | 5 | 2 | 2 | 4 | 5 | 5 | 5 | 1 | 2 | 2 | 2 |
| 1.079 | 5 | 5 | 2 | 4 | 5 | 5 | 5 | 5 | 1 | 5 | 5 | 4 |
| 1.080 | 5 | 5 | 5 | 5 | 4 | 5 | 5 | 5 | 5 | 5 | 5 | 5 |
| 1.081 | 5 | 5 | 4 | 5 | 4 | 5 | 5 | 5 | 2 | 5 | 5 | 5 |
| 1.082 | 5 | 5 | 2 | 5 | 5 | 5 | 5 | 5 | 3 | 5 | 5 | 5 |
| 1.083 | 5 | 5 | 5 | 5 | 5 | 5 | 5 | 5 | 5 | 5 | 5 | 5 |
| 1.084 | 5 | 5 | 2 | 5 | 5 | 5 | 5 | 5 | 2 | 5 | 5 | 5 |
| 1.085 | 5 | 5 | 2 | 5 | 5 | 5 | 5 | 5 | 2 | 5 | 5 | 5 |
| 1.086 | 5 | 5 | 2 | 5 | 5 | 5 | 5 | 5 | 2 | 5 | 5 | 5 |
| 1.087 | 5 | 5 | 5 | 5 | 5 | 5 | 5 | 5 | 5 | 5 | 5 | 5 |
| 1.088 | 5 | 5 | 5 | 5 | 5 | 5 | 5 | 5 | 5 | 5 | 5 | 5 |
| 1.089 | 5 | 5 | 5 | 5 | 5 | 5 | 5 | 5 | 5 | 5 | 5 | 5 |
| 1.090 | 5 | 5 | 5 | 5 | 5 | 5 | 5 | 5 | 5 | 5 | 5 | 5 |
| 1.091* | 5 | 5 | 4 | 4 | 5 | 5 | 5 | 5 | 4 | 4 | 5 | 4 |
| 1.092 | 5 | 5 | 5 | 4 | 5 | 5 | 5 | 5 | 5 | 4 | 5 | 5 |
| 1.093 | 5 | 5 | 5 | 5 | 5 | 5 | 5 | 5 | 5 | 5 | 5 | 5 |
| 1.094 | 5 | 5 | 5 | 5 | 5 | 5 | 5 | 5 | 5 | 5 | 5 | 5 |
| 1.095 | 5 | 5 | 5 | 5 | 5 | 5 | 5 | 5 | 5 | 5 | 5 | 5 |
| 1.096 | 5 | 5 | 5 | 4 | 5 | 5 | 5 | 5 | 4 | 5 | 5 | 5 |
| 2.001 | 4 | 4 | 1 | 1 | 1 | 1 | 1 | 1 | 1 | 1 | 1 | 1 |
| 2.003 | 2 | 1 | 1 | 1 | 1 | 2 | 4 | 2 | 3 | 1 | 1 | 5 |
| 4.003 | 5 | 5 | 1 | 2 | 1 | 2 | 4 | 5 | 1 | 2 | 1 | 3 |
| 4.004 | 5 | 5 | 1 | 1 | 1 | 4 | 2 | 5 | 1 | 1 | 1 | 2 |
| 4.005 | 5 | 5 | 1 | 3 | 2 | 5 | 5 | 4 | 1 | 1 | 2 | 4 |
| 5.001 | 5 | 5 | 5 | 5 | 5 | 5 | 5 | 5 | 1 | 1 | 1 | 4 |
| 5.002 | 5 | 5 | 5 | 5 | 5 | 5 | 5 | 5 | 2 | 3 | 3 | 5 |
| 5.003 | 5 | 5 | 4 | 3 | 4 | 4 | 5 | 5 | 4 | 1 | 1 | 4 |
| 6.001 | 5 | 5 | 3 | 3 | 5 | 5 | 5 | 5 | 5 | 1 | 1 | 1 |
| 6.002 | 5 | 5 | 4 | 4 | 4 | 5 | 5 | 5 | 2 | 2 | 2 | 5 |
| 6.003 | 4 | 5 | 2 | 3 | 1 | 5 | 5 | 5 | 5 | 1 | 4 | 5 |

*Applied at 250 g/ha.

1. A compound of Formula (I): 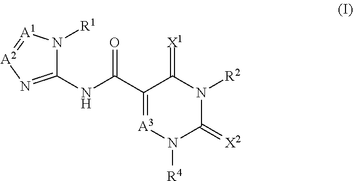
or an agronomically acceptable salt thereof,
wherein:—
X1 and X2 are independently selected from the group consisting of O and S;
A1 and A2 are independently selected from CH and N, wherein A1 and A2 are not both CH;
A3 is N or CR5;
wherein
R1 is selected from the group consisting of hydrogen, C1-C6alkyl-, C1-C6 haloalkyl- and C1-C6alkoxy-C1-C3alkyl-;
R2 is selected from the group consisting of hydrogen, C1-C6alkyl-, C1-C6haloalkyl-, C1-C3alkoxy-C1-C3alkyl-, C1-C3alkoxy-C2-C3alkoxy-C1-C3-alkyl-, C1-C3haloalkoxy-C1-C3-alkyl-, (C1-C3 alkylsulfonyl-C1-C3 alkylamino)-C1-C3alkyl-, (C1-C3alkylsulfonyl-C3-C4cycloalkylamino)-C1-C3alkyl-, C1-C6alkylcarbonyl-C1-C3alkyl-, C3-C6cycloalkyl-C2-C6alkenyl-, C2-C6alkynyl-, C2-C6-alkenyl-, C2-C6-haloalkenyl-, cyano-C1-C6-alkyl-, arylcarbonyl-C1-C3-alkyl-, aryl-C1-C6alkyl-, aryloxy-C1-C6alkyl (wherein said aryl groups may be optionally substituted with one or more substituents from the group consisting of halo, C1-C3-alkoxy, C1-C3-alkyl, C1-C3 haloalkyl), and a three- to ten-membered mono- or bicyclic ring system, which may be aromatic, saturated or partially saturated and can contain from 1 to 4 heteroatoms each independently selected from the group consisting of nitrogen, oxygen and sulphur the ring system being optionally substituted by one or more substituents selected from the group consisting of nitro, cyano, halogen, C1-C3alkyl-, C1-C3haloalkyl-, C2-C3alkenyl-, C2-C3alkynyl-, C1-C3alkoxy-, C1-C3alkoxy-C1-C3alkyl-, C1-C3alkoxy-C2-C3alkoxy-, C1-C3alkoxy-C2-C3alkoxy-C1-C3-alkyl-, C1-C3 haloalkoxy, C1-C6alkyl-S(0)p- and C1-C6haloalkyl-S(0)p-;
R4 is selected from the group consisting of hydrogen, C1-C6alkyl-, C1-C6haloalkyl-, C1-C6alkoxy-C1-C6alkyl-, C1-C6haloalkoxy-C1-C6alkyl-, C1-C6alkoxy-C1-C6alkoxy-C1-C6alkyl-, C2-C6alkenyl-, C2-C6haloalkenyl-, C2-C6alkynyl-, C2-C6haloalkynyl- and C3-C6 cycloalkyl-;
R5 is selected from the group consisting of hydrogen, hydroxyl, C1-C6alkyl-, C1-C6 haloalkyl- and C1-C6alkoxy-C1-C3alkyl-; and
p=0, 1 or 2.

2. The compound according to claim 1, wherein A1 and A2 are both N.

3. The compound according to claim 1, wherein X1 and X2 are oxygen.

4. The compound according to claim 1, wherein R1 is methyl or ethyl.

5. The compound according to claim 1, wherein R2 is selected from the group consisting of C1 -C6alkyl-, C1-C3alkoxy-C1-C3alkyl-, C1-C3haloalkoxy-C1-C3alkyl-, phenyl and a 5 or 6 membered heteroaromatic ring system containing from 1 to 4 heteroatoms each independently selected from the group consisting of nitrogen, oxygen and sulphur;
the phenyl or 5 or 6 heteroaromatic ring system being optionally substituted by one or more substituents selected from the group consisting of nitro, cyano, halogen, C1-C3alkyl, C1-C3haloalkyl, C2-C3alkenyl, C2-C3alkynyl, C1-C3 alkoxy, C1-C3 haloalkoxy, C1-C6alkyl-S(O)p- and C1-C6haloalkyl-S(O)p-.

6. The compound according to claim 1, wherein R2 is selected from the group consisting of C1 -C6alkyl-, C1-C3alkoxy-C1-C3alkyl-, C1-C6alkylcarbonyl-C1-C3alkyl, benzyl-, pyridyl and phenyl-, the benzyl, pyridyl and phenyl groups being optionally substituted by one or more substituents selected from the group consisting of cyano, halogen, C1-C3alkyl-, C1-C3haloalkyl-, C2-C3alkenyl-, C1-C3alkoxy- and C1-C3haloalkoxy-.

7. The compound according to claim 1, wherein R4 is methyl.

8. The compound according to claim 1, wherein R5 is hydrogen.

9. A herbicidal composition comprising a compound of Formula (I) according to claim 1 and an agriculturally acceptable formulation adjuvant.

10. The herbicidal composition according to claim 9, further comprising at least one additional pesticide.

11. The herbicidal composition according to claim 10, wherein the additional pesticide is a herbicide or herbicide safener.

12. (canceled)

13. (canceled)

14. A compound of Formula (II) 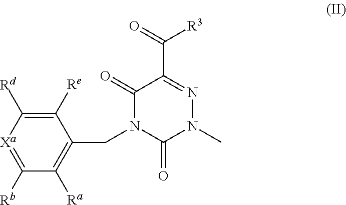
wherein
Xa=CRc or N;
Ra, Rb, Rc, Rd and Re are independently selected from the group consisting of nitro, cyano, halogen, C1-C3alkyl, C1-C3haloalkyl, C2-C3alkenyl, C2-C3alkynyl, C1-03 alkoxy, C1-C3alkoxy-C1-C3alkyl-, C1-C3alkoxy-C2-C3alkoxy-, C1-C3alkoxy-C2-C3alkoxy-C1-C3-alkyl-, C1-C3 haloalkoxy, C1-C6alkyl-S(O)p- and C1-C6haloalkyl-S(O)p-;
R3=hydroxyl or C1-C3 alkoxy; and
p=0, 1 or 2.
